SEQUENCE LISTING (135 sequences)

SEQ ID NO: 1 - protein, 293 aa, Homo sapiens
MDAPHSKAALDSINELPENILLELFTHVPARQLLLNCRLVCSLWRDLIDLMTLWKRKCLREGFITKDWDQPVADWKIFYFLRSLHRNLLRNPCAEEDMFAWQIDFNGGDRWKVESLPGAHGTDFPDPKVKKYFVTSYEMCLKSQLVDLVAEGYWEELLDTFRPDIVVKDWFAARADCGCTYQLKVQLASADYFVLASFEPPPVTIQQWNNATWTEVSYTFSDYPRGVRYILFQHGGRDTQYWAGWYGPRVTNSSIVVSPKMTRNQASSEAQPGQKHGQEEAAQSPYRAVVQIF

SEQ ID NO: 2 - protein, 451 aa, Homo sapiens
MPKNKKRNTPHRGSSAGGGGSGAAAATAATAGGQHRNVQPFSDEDASIETMSHCSGYSDPSSFAEDGPEVLDEEGTQEDLEYKLKGLIDLTLDKSAKTRQAALEGIKNALASKMLYEFILERRMTLTDSIERCLKKGKSDEQRAAAALASVLCIQLGPGIESEEILKTLGPILKKIICDGSASMQARQTCATCFGVCCFIATDDITELYSTLECLENIFTKSYLKEKDTTVICSTPNTVLHISSLLAWTLLLTICPINEVKKKLEMHFHKLPSLLSCDDVNMRIAAGESLALLFELARGIESDFFYEDMESLTQMLRALATDGNKHRAKVDKRKQRSVFRDVLRAVEERDFPTETIKFGPERMYIDCWVKKHTYDTFKEVLGSGMQYHLQSNEFLRNVFELGPPVMLDAATLKTMKISRFERHLYNSAAFKARTKARSKCRDKRADVGEFF

SEQ ID NO: 3 - protein, 900 aa, Homo sapiens
MRLFVSDGVPGCLPVLAAAGRARGRAEVLISTVGPEDCVVPFLTRPKVPVLQLDSGNYLFSTSAICRYFFLLSGWEQDDLTNQWLEWEATELQPALSAALYYLVVQGKKGEDVLGSVRRALTHIDHSLSRQNCPFLAGETESLADIVLWGALYPLLQDPAYLPEELSALHSWFQTLSTQEPCQRAAETVLKQQGVLALRPYLQKQPQPSPAEGRAVTNEPEEEELATLSEEEIAMAVTAWEKGLESLPPLRPQQNPVLPVAGERNVLITSALPYVNNVPHLGNIIGCVLSADVFARYSRLRQWNTLYLCGTDEYGTATETKALEEGLTPQEICDKYHIIHADIYRWFNISFDIFGRTTTPQQTKITQDIFQQLLKRGFVLQDTVEQLRCEHCARFLADRFVEGVCPFCGYEEARGDQCDKCGKLINAVELKKPQCKVCRSCPVVQSSQHLFLDLPKLEKRLEEWLGRTLPGSDWTPNAQFITRSWLRDGLKPRCITRDLKWGTPVPLEGFEDKVFYVWFDATIGYLSITANYTDQWERWWKNPEQVDLYQFMAKDNVPFHSLVFPCSALGAEDNYTLVSHLIATEYLNYEDGKFSKSRGVGVFGDMAQDTGIPADIWRFYLLYIRPEGQDSAFSWTDLLLKNNSELLNNLGNFINRAGMFVSKFFGGYVPEMVLTPDDQRLLAHVTLELQHYHQLLEKVRIRDALRSILTISRHGNQYIQVNEPWKRIKGSEADRQRAGTVTGLAVNIAALLSVMLQPYMPTVSATIQAQLQLPPPACSILLTNFLCTLPAGHQIGTVSPLFQKLENDQIESLRQRFGGGQAKTSPKPAVVETVTTAKPQQIQALMDEVTKQGNIVRELKAQKADKNEVAAEVAKLLDLKKQLAVAEGKPPEAPKGKKKK

SEQ ID NO: 4 - protein, 959 aa, Homo sapiens
MAASTGYVRLWGAARCWVLRRPMLAAAGGRVPTAAGAWLLRGQRTCDASPPWALWGRGPAIGGQWRGFWEASSRGGGAFSGGEDASEGGAEEGAGGAGGSAGAGEGPVITALTPMTIPDVFPHLPLIAITRNPVFPRFIKIIEVKNKKLVELLRRKVRLAQPYVGVFLKRDDSNESDVVESLDEIYHTGTFAQIHEMQDLGDKLRMIVMGHRRVHISRQLEVEPEEPEAENKHKPRRKSKRGKKEAEDELSARHPAELAMEPTPELPAEVLMVEVENVVHEDFQVTEEVKALTAEIVKTIRDIIALNPLYRESVLQMMQAGQRVVDNPIYLSDMGAALTGAESHELQDVLEETNIPKRLYKALSLLKKEFELSKLQQRLGREVEEKIKQTHRKYLLQEQLKIIKKELGLEKDDKDAIEEKFRERLKELVVPKHVMDVVDEELSKLGLLDNHSSEFNVTRNYLDWLTSIPWGKYSNENLDLARAQAVLEEDHYGMEDVKKRILEFIAVSQLRGSTQGKILCFYGPPGVGKTSIARSIARALNREYFRFSVGGMTDVAEIKGHRRTYVGAMPGKIIQCLKKTKTENPLILIDEVDKIGRGYQGDPSSALLELLDPEQNANFLDHYLDVPVDLSKVLFICTANVTDTIPEPLRDRMEMINVSGYVAQEKLAIAERYLVPQARALCGLDESKAKLSSDVLTLLIKQYCRESGVRNLQKQVEKVLRKSAYKIVSGEAESVEVTPENLQDFVGKPVFTVERMYDVTPPGVVMGLAWTAMGGSTLFVETSLRRPQDKDAKGDKDGSLEVTGQLGEVMKESARIAYTFARAFLMQHAPANDYLVTSHIHLHVPEGATPKDGPSAGCTIVTALLSLAMGRPVRQNLAMTGEVSLTGKILPVGGIKEKTIAAKRAGVTCIVLPAENKKDFYDLAAFITEGLEVHFVEHYREIFDIAFPDEQAEALAVER

SEQ ID NO: 5 - protein, 312 aa, Homo sapiens
MEEECRVLSIQSHVIRGYVGNRAATFPLQVLGFEIDAVNSVQFSNHTGYAHWKGQVLNSDELQELYEGLRLNNMNKYDYVLTGYTRDKSFLAMVVDIVQELKQQNPRLVYVCDPVLGDKWDGEGSMYVPEDLLPVYKEKVVPLADIITPNQFEAELLSGRKIHSQEEALRVMDMLHSMGPDTVVITSSDLPSPQGSNYLIVLGSQRRRNPAGSVVMERIRMDIRKVDAVFVGTGDLFAAMLLAWTHKHPNNLKVACEKTVSTLHHVLQRTIQCAKAQAGEGVRPSPMQLELRMVQSKRDIEDPEIVVQATVL

SEQ ID NO: 6 - protein, 665 aa, Homo sapiens
MAAGGSTQQRRREMAAASAAAISGAGRCRLSKIGATRRPPPARVRVAVRLRPFVDGTAGASDPPCVRGMDSCSLEIANWRNHQETLKYQFDAFYGERSTQQDIYAGSVQPILRHLLEGQNASVLAYGPTGAGKTHTMLGSPEQPGVIPRALMDLLQLTREEGAEGRPWALSVTMSYLEIYQEKVLDLLDPASGDLVIREDCRGNILIPGLSQKPISSFADFERHFLPASRNRTVGATRLNQRSSRSHAVLLVKVDQRERLAPFRQREGKLYLIDLAGSEDNRRTGNKGLRLKESGAINTSLFVLGKVVDALNQGLPRVPYRDSKLTRLLQDSLGGSAHSILIANIAPERRFYLDTVSALNFAARSKEVINRPFTNESLQPHALGPVKLSQKELLGPPEAKRARGPEEEEIGSPEPMAAPASASQKLSPLQKLSSMDPAMLERLLSLDRLLASQGSQGAPLLSTPKRERMVLMKTVEEKDLEIERLKTKQKELEAKMLAQKAEEKENHCPTMLRPLSHRTVTGAKPLKKAVVMPLQLIQEQAASPNAEIHILKNKGRKRKLESLDALEPEEKAEDCWELQISPELLAHGRQKILDLLNEGSARDLRSLQRIGPKKAQLIVGWRELHGPFSQVEDLERVEGITGKQMESFLKANILGLAAGQRCGAS

SEQ ID NO: 7 - protein, 320 aa, Homo sapiens
MEVEAVCGGAGEVEAQDSDPAPAFSKAPGSAGHYELPWVEKYRPVKLNEIVGNEDTVSRLEVFAREGNVPNIIIAGPPGTGKTTSILCLARALLGPALKDAMLELNASNDSMTDGAQQALRRTMEIYSKTTRFALACNASDKIIEPIQSRCAVLRYTKLTDAQILTRLMNVIEKERVPYTDDGLEAIIFTAQGDMRQALNNLQSTFSGFGFINSENVFKVCDEPHPLLVKEMIQHCVNANIDEAYKILAHLWHLGYSPEDIIGNIFRVCKTFQMAEYLKLEFIKEIGYTHMKIAEGVNSLLQMAGLLARLCQKTMAPVAS

SEQ ID NO: 8 - protein, 298 aa, Homo sapiens [sequence omitted]

SEQ ID NO: 9 - protein, 296 aa, Homo sapiens
MATSSAALPRILGAGARAPSRWLGFLGKATPRPARPSRRTLGSATALMIRESEDSTDFNDKILNEPLKHSDFFNVKELFSVRSLFDARVHLGHKAGCRHRFMEPYIFGSRLDHDIIDLEQTATHLQLALNFTAHMAYRKGIILFISRNRQFSYLIENMARDCGEYAHTRYFRGGMLTNARLLFGPTVRLPDLIIFLHTLNNIFEPHVAVRDAAKMNIPTVGIVDTNCNPCLITYPVPGNDDSPLAVHLYCRLFQTAITRAKEKRQQVEALYRLQGQKEPGDQGPAHPPGADMSHSL

SEQ ID NO: 10 - protein, 937 aa, Homo sapiens [sequence omitted]

SEQ ID NO: 11 - protein, 441 aa, Homo sapiens
MCVCQTMEVGQYGKNASRAGDRGVLLEPFIHQVGGHSSMMRYDDHTVCKPLISREQRFYESLPPEMKEFTPEYKGVVSVCFEGDSDGYINLVAYPYVESETVEQDDTTEREQPRRKHSRRSLHRSGSGSDHKEEKASLSLETSESSQEAKSPKVELHSHSEVPFQMLDGNSGLSSEKISHNPWSLRCHKQQLSRMRSESKDRKLYKFLLLENVVHHFKYPCVLDLKMGTRQHGDDASAEKAARQMRKCEQSTSATLGVRVCGMQVYQLDTGHYLCRNKYYGRGLSIEGFRNALYQYLHNGLDLRRDLFEPILSKLRGLKAVLERQASYRFYSSSLLVIYDGKECRAESCLDRRSEMRLKHLDMVLPEVASSCGPSTSPSNTSPEAGPSSQPKVDVRMIDFAHSTFKGFRDDPTVHDGPDRGYVFGLENLISIMEQMRDENQ

SEQ ID NO: 12 - protein, 286 aa, Homo sapiens [sequence omitted]

SEQ ID NO: 13 - protein, 639 aa, Homo sapiens
MNLEKLSKPELLTLFSILEGELEARDLVIEALKAQHRDTFIEERYGKYNISDPLMALQRDFETLKEKNDGEKQPVCTNPLSILKVVMKQCKNMQERMLSQLAAAESRHRKVILDLEEERQRHAQDTAEGDDVTYMLEKERERLTQQLEFEKSQVKKFEKEQKKLSSQLEEERSRHKQLSSMLVLECKKATNKAAEEGQKAGELSLKLEKEKSRVSKLEEELAAERKRGLQTEAQVEKQLSEFDIEREQLRAKLNREENRTKTLKEEMESLKKIVKDLEASHQHSSPNEQLKKPVTVSKGTATEPLMLMSVFCQTESFPAERTHGSNIAKMTNTGLPGPATPAYSYAKTNGHCDPEIQTTRELTAGNNVENQVPPREKSVALAQEKPVENGGCPVGIETPVPMPSPLSSSGSSLSPSSTASSSLTSSPCSSPVLTKRLLGSSASSPGYQSSYQVGINQRFHAARHKFQSQADQDQQASGLQSPPSRDLSPTLIDNSAAKQLARNTVTQVLSRFTSQQGPIKPVSPNSSPFGTDYRNLANTANPRGDTSHSPTPGKVSSPLSPLSPGIKSPTIPRAERGNPPPIPPKKPGLTPSPSATTPLTKTHSQAASLTTAEDLASSCSSNTVVANGKDVELLLPTSS

SEQ ID NO: 14 - protein, 308 aa, Homo sapiens
MCSCSLTPVPEPVLREGAEGLHCSEHLLLEKAPSYGSEGPAQRVLEGTLLEFTKDMDQLLQLTRKLESSQPRQGEGNRTPESQKRKSKKATKQTLQDSFLLDLKSPPSFPVEISDRLPAASWEGQESCWNKQTSKSEGTQPEGTYGEQLVPELCNQSESSGEDFFLKSRLQEQDVWRRSTSFYTHMCNPWVSLLGAVGSLLIMFVIQWVYTLVNMGVAAIVYFYIGRASPGLHLGSASNFSFFRWMRSLLLPSCRSLQSPQEQIILAPSLAKVDMEMTQLTQENADFATRDRYHHSSLVNREQLMPHY

SEQ ID NO: 15 - protein, 911 aa, Homo sapiens [sequence omitted]

SEQ ID NO: 16 - protein, 438 aa, Homo sapiens
MVQWKRLCQLHYLWALGCYMLLATVALKLSFRLKCDSDHLGLESRESQSQYCRNILYNFLKLPAKRSINCSGVTRGDQEAVLQAILNNLEVKKKREPFTDTHYLSLTRDCEHFKAERKFIQFPLSKEEVEFPIAYSMVIHEKIENFERLLRAVYAPQNIYCVHVDEKSPETFKEAVKAIISCFPNVFIASKLVRVVYASWSRVQADLNCMEDLLQSSVPWKYFLNTCGTDFPIKSNAEMVQALKMLNGRNSMESEVPPKHKETRWKYHFEVVRDTLHLTNKKKDPPPYNLTMFTGNAYIVASRDFVQHVLKNPKSQQLIEWVKDTYSPDEHLWATLQRARWMPGSVPNHPKYDISDMTSIARLVKWQGHEGDIDKGAPYAPCSGIHQRAICVYGAGDLNWMLQNHHLLANKFDPKVDDNALQCLEEYLRYKAIYGTEL

SEQ ID NO: 17 - protein, 445 aa, Homo sapiens
MTELRQRVAHEPVAPPEDKESESEAKVDGETASDSESRAESAPLPVSADDTPEVLNRALSNLSSRWKNWWVRGILTLAMIAFFFIIIYLGPMVLMIIVMCVQIKCFHEIITIGYNVYHSYDLPWFRTLSWYFLLCVNYFFYGETVTDYFFTLVQREEPLRILSKYHRFISFTLYLIGFCMFVLSLVKKHYRLQFYMFGWTHVTLLIVVTQSHLVIHNLFEGMIWFIVPISCVICNDIMAYMFGFFFGRTPLIKLSPKKTWEGFIGGFFATVVFGLLLSYVMSGYRCFVCPVEYNNDTNSFTVDCEPSDLFRLQEYNIPGVIQSVIGWKTVRMYPFQIHSIALSTFASLIGPFGGFFASGFKRAFKIKDFANTIPGHGGIMDRFDCQYLMATFVNVYIASFIRGPNPSKLIQQFLTLRPDQQLHIFNTLRSHLIDKGMLTSTTEDE

SEQ ID NO: 18 - protein, 305 aa, Homo sapiens
MAAAGQEKGYLTQTAAALDKSPSLSPQLAAPIRGRPKKCLVYPHAPKSSRLSRSVLRWLQGLDLSFFPRDINRDFSNGFLIAEIFCIYYPWELELSSFENGTSLKVKLDNWAQLEKFLARKKFKLPKELIHGTIHCKAGVPEILIEEVYTLLTHREIKSIQDDFVNFTDYSYQMRLPLVSRSTVSKSIKDNIRLSELLSNPNMLTNELKAEFLILLHMLQRKLGRKLNPEWFDVKPTVGEVTLNHLPAQASGRRYNLKVKRGRVVPVLPNIGSGGSSHREIHVKQAGQHSYYSAMKPIRNMDKKP

SEQ ID NO: 19 - protein, 643 aa, Homo sapiens
MVDAGGVENITQLPQELPQMMAAAADGLGSIAIDTTQLNMSVTDPTAWATAMNNLGMVPVGLPGQQLVSDSICVPGFDPSLNMMTGITPINPMIPGLGLVPPPPPTEVAVVKEIIHCKSCTLFPQNPNLPPPSTRERPPGCKTVFVGGLPENATEEIIQEVFEQCGDITAIRKSKKNFCHIRFAEEFMVDKAIYLSGYRMRLGSSTDKKDSGRLHVDFAQARDDFYEWECKQRMRAREERHRRKLEEDRLRPPSPPAIMHYSEHEAALLAEKLKDDSKFSEAITVLLSWIERGEVNRRSANQFYSMVQSANSHVRRLMNEKATHEQEMEEAKENFKNALTGILTQFEQIVAVFNASTRQKAWDHFSKAQRKNIDIWRKHSEELRNAQSEQLMGIRREEEMEMSDDENCDSPTKKMRVDESALAAQAYALKEENDSLRWQLDAYRNEVELLKQEKEQLFRTEENLTKDQQLQFLQQTMQGMQQQLLTIQEELNNKKSELEQAKEEQSHTQALLKVLQEQLKGTKELVETNGHSHEDSNEINVLTVALVNQDRENNIEKRSQGLKSEKEALLIGIISTFLHVHPFGANIEYLWSYMQQLDSKISANEIEMLLMRLPRMFKQEFTGVGATLEKRWKLCAFEGIKTT

SEQ ID NO: 20 - protein, 472 aa, Homo sapiens
MAGGEAGVTLGQPHLSRQDLTTLDVTKLTPLSHEVISRQATINIGTIGHVAHGKSTVVKAISGVHTVRFKNELERNITIKLGYANAKIYKLDDPSCPRPECYRSCGSSTPDEFPTDIPGTKGNFKLVRHVSFVDCPGHDILMATMLNGAAVMDAALLLIAGNESCPQPQTSEHLAAIEIMKLKHILILQNKIDLVKESQAKEQYEQILAFVQGTVAEGAPIIPISAQLKYNIEVVCEYIVKKIPVPPRDFTSEPRLIVIRSFDVNKPGCEVDDLKGGVAGGSILKGVLKVGQEIEVRPGIVSKDSEGKLMCKPIFSKIVSLFAEHNDLQYAAPGGLIGVGTKIDPTLCRADRMVGQVLGAVGALPEIFTELEISYFLLRRLLGVRTEGDKKAAKVQKLSKNEVLMVNIGSLSTGGRVSAVKADLGKIVLTNPVCTEVGEKIALSRRVEKHWRLIGWGQIRRGVTIKPTVDDD

SEQ ID NO: 21 - protein, 446 aa, Homo sapiens
MAAATIVHDTSEAVELCPAYGLYLKPITKMTISVALPQLKQPGKSISNWEVMERLKGMVQNHQFSTLRISKSTMDFIRFEGEVENKSLVKSFLACLDGKTIKLSGFSDILKVRAAEFKIDFPTRHDWDSFFRDAKDMNETLPGERPDTIHLEGLPCKWFALKESGSEKPSEDVLVKVFEKFGEIRNVDIPMLDSYREEMTGRNFHTFSFGGHLNFEAYVQYREYMGFIQAMSALRGMKLMYKGEDGKAVACNIKVSFDSTKHLSDASIKKRQLERQKLQELEQQREEQKRREKEAEERQRAEERKQKELEELERERKREEKLRKREQKQRDRELRRNQKKLEKLQAEEQKQLQEKIKLEERKLLLAQRNLQSIRLIAELLSRAKVPGGSLCSRQPRPGCPQCPPLKCGRRHGAVSPPAAAVATKPALMPRMTAPSREGVALVCRSR

SEQ ID NO: 22 - protein, 317 aa, Homo sapiens
MPWDARRPGGGADGGPEASGAARSRAQKQCRKSSFAFYQAVRDLLPVWLLEDMRASEAFHWDERGRAAAYSPSEALLYALVHDHQAYAHYLLATFPRRALAPPSAGFRCCAAPGPHVALAVRYNRVGILRRILRTLRDFPAEERARVLDRRGCSRVEGGGTSLHVACELARPECLFLLLGHGASPGLRDGGGLTPLELLLRQLGRDAGATPSAAGAPASAPGEPRQRRLLLLDLLALYTPVGAAGSARQELLGDRPRWQRLLGEDKFQWLAGLAPPSLFARAMQVLVTAISPGRFPEALDELPLPPFLQPLDLTGKG

SEQ ID NO: 23 - protein, 913 aa, Homo sapiens
MSRFFTTGSDSESESSLSGEELVTKPVGGNYGKQPLLLSEDEEDTKRVVRSAKDKRFEELTNLIRTIRNAMKIRDVTKCLEEFELLGKAYGKAKSIVDKEGVPRFYIRILADLEDYLNELWEDKEGKKKMNKNNAKALSTLRQKIRKYNRDFESHITSYKQNPEQSADEDAEKNEEDSEGSSDEDEDEDGVSAATFLKKKSEAPSGESRKFLKKMDDEDEDSEDSEDDEDWDTGSTSSDSDSEEEEGKQTALASRFLKKAPTTDEDKKAAEKKREDKAKKKHDRKSKRLDEEEEDNEGGEWERVRGGVPLVKEKPKMFAKGTEITHAVVIKKLNEILQARGKKGTDRAAQIELLQLLVQIAAENNLGEGVIVKIKFNIIASLYDYNPNLATYMKPEMWGKCLDCINELMDILFANPNIFVGENILEESENLHNADQPLRVRGCILTLVERMDEEFTKIMQNTDPHSQEYVEHLKDEAQVCAIIERVQRYLEEKGTTEEVCRIYLLRILHTYYKFDYKAHQRQLTPPEGSSKSEQDQAENEGEDSAVLMERLCKYIYAKDRTDRIRTCAILCHIYHHALHSRWYQARDLMLMSHLQDNIQHADPPVQILYNRTMVQLGICAFRQGLTKDAHNALLDIQSSGRAKELLGQGLLLRSLQERNQEQEKVERRRQVPFHLHINLELLECVYLVSAMLLEIPYMAAHESDARRRMISKQFHHQLRVGERQPLLGPPESMREHVVAASKAMKMGDWKTCHSFIINEKMNGKVWDLFPEADKVRTMLVRKIQEESLRTYLFTYSSVYDSISMETLSDMFELDLPTVHSIISKMIINEELMASLDQPTQTVVMHRTEPTAQQNLALQLAEKLGSLVENNERVFDHKQGTYGGYFRDQKDGYRKNEGYMRRGGYRQQQSQTAY

SEQ ID NO: 24 - protein, 992 aa, Homo sapiens
MGAAGRQDFLFKAMLTISWLTLTCFPGATSTVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDKWHSLASKAASGPSG

SEQ ID NO: 25 - protein, 647 aa, Homo sapiens
MGEIEGTYRVLQTAGMRLGAQTPVGVSTLEPGQTLLPRMQEKHLHLRVKLLDNTMEIFDIEPKCDGQVLLTQVWKRLNLVECDYFGMEFQNTQSYWIWLEPMKPIIRQIRRPKNVVLRLAVKFFPPDPGQLQEEYTRYLFALQLKRDLLEERLTCADTTAALLTSHLLQSEIGDYDETLDREHLKVNEYLPGQQHCLEKILEFHQKHVGQTPAESDFQVLEIARKLEMYGIRFHMASDREGTKIQLAVSHMGVLVFQGTTKINTFNWSKVRKLSFKRKRFLIKLHPEVHGPYQDTLEFLLGSRDECKNFWKICVEYHTFFRLLDQPKPKAKAVFFSRGSSFRYSGRTQKQLVDYFKDSGMKRIPYERRHSKTHTSVRALTADLPKQSISFPEGLRTPASPSSANAFYSLSPSTLVPSGLPEFKDSSSSLTDPQVSYVKSPAAERRSGAVAGGPDTPSAQPLGPPALQPGPGLSTKSPQPSPSSRKSPLSLSPAFQVPLGPAEQGSSPLLSPVLSDAGGAGMDCEEPRHKRVPADEAYFIVKEILATERTYLKDLEVITVWFRSAVVKEDAMPATLMTLLFSNIDPIYEFHRGFLREVEQRLALWEGPSKAHTKGSHQRIGDILLRNMRQLKVFQLHEGHVAGVTKME

SEQ ID NO: 26 - protein, 304 aa, Homo sapiens
MASETHNVKKRNFCNKIEDHFIDLPRKKISNFTNKNMKEVKKSPKQLAAYINRTVGQTVKSPDKLRKVIYRRKKVHHPFPNPCYRKKQSPGSGGCDMANKENELACAGHLPEKLHHDSRTYLVNSSDSGSSQTESPSSKYSGFFSEVSQDHETMAQVLFSRNMRLNVALTFWRKRSISELVAYLLRIEDLGVVVDCLPVLTNCLQEEKQYISLGCCVDLLPLVKSLLKSKFEEYVIVGLNWLQAVIKRWWSELSSKTEIINDGNIQILKQQLSGLWEQENHLTLVPGYTGNIAKDVDAYLLQLH

SEQ ID NO: 27 - protein, 304 aa, Homo sapiens
METDESPSPLPCGPAGEAVMESRARPFQALPREQSPPPPLQTSSGAEVMDVGSGGDGQSELPAEDPFNFYGASLLSKGSFSKGRLLIDPNCSGHSPRTARHAPAVRKFSPDLKLLKDVKISVSFTESCRSKDRKVLYTGAERDVRAECGLLLSPVSGDVHACPFGGSVGDGVGIGGESADKKDEENELDQEKRVEYAVLDELEDFTDNLELDEEGAGGFTAKAIVQRDRVDEEALNFPYEDDFDNDVDALLEEGLCAPKKRRTEEKYGGDSDHPSDGETSVQPMMTKIKTVLKSRGRPPTEPVL

SEQ ID NO: 28 - protein, 459 aa, Homo sapiens
MAASASAAAGEEDWVLPSEVEVLESIYLDELQVIKGNGRTSPWEIYITLHPATAEDQDSQYVCFTLVLQVPAEYPHEVPQISIRNPRGLSDEQIHTILQVLGHVAKAGLGTAMLYELIEKGKEILTDNNIPHGQCVICLYGFQEKEAFTKTPCYHYFHCHCLARYIQHMEQELKAQGQEQEQERQHATTKQKAVGVQCPVCREPLVYDLASLKAAPEPQQPMELYQPSAESLRQQEERKRLYQRQQERGGIIDLEAERNRYFISLQQPPAPAEPESAVDVSKGSQPPSTLAAELSTSPAVQSTLPPPLPVATQHICEKIPGTRSNQQRLGETQKAMLDPPKPSRGPWRQPERRHPKGGECHAPKGTRDTQELPPPEGPLKEPMDLKPEPHSQGVEGPPQEKGPGSWQGPPPRRTRDCVRWERSKGRTPGSSYPRLPRGQGAYRPGTRRESLGLESKDGS

SEQ ID NO: 29 - protein, 295 aa, Homo sapiens
MMAKKPPKPAPRRIFQERLKITALPLYFEGFLLIKRSGYREYEHYWTELRGTTLFFYTDKKSIIYVDKLDIVDLTCLTEQNSTEKNCAKFTLVLPKEEVQLKTENTESGEEWRGFILTVTELSVPQNVSLLPGQVIKLHEVLEREKKRRIETEQSTSVEKEKEPTEDYVDVLNPMPACFYTVSRKEATEMLQKNPSLGNMILRPGSDSRNYSITIRQEIDIPRIKHYKVMSVGQNYTIELEKPVTLPNLFSVIDYFVKETRGNLRPFICSTDENTGQEPSMEGRSEKLKKNPHIA

SEQ ID NO: 30 - protein, 315 aa, Homo sapiens
MEARAQSGNGSQPLLQTPRDGGRQRGEPDPRDALTQQVHVLSLDQIRAIRNTNEYTEGPTVVPRPGLKPAPRPSTQHKHERLHGLPEHRQPPRLQHSQVHSSARAPLSRSISTVSSGSRSSTRTSTSSSSSEQRLLGSSFSSGPVADGIIRVQPKSELKPGELKPLSKEDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT

SEQ ID NO: 31 - protein, 662 aa, Homo sapiens
MSHVAVENALGLDQQFAGLDLNSSDNQSGGSTASKGRYIPPHLRNREATKGFYDKDSSGWSSSKDKDAYSSFGSRSDSRGKSSFFSDRGSGSRGRFDDRGRSDYDGIGSRGDRSGFGKFERGGNSRWCDKSDEDDWSKPLPPSERLEQELFSGGNTGINFEKYDDIPVEATGNNCPPHIESFSDVEMGEIIMGNIELTRYTRPTPVQKHAIPIIKEKRDLMACAQTGSGKTAAFLLPILSQIYSDGPGEALRAMKENGRYGRRKQYPISLVLAPTRELAVQIYEEARKFSYRSRVRPCVVYGGADIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEESDKRSFLLDLLNATGKDSLTLVFVETKKGADSLEDFLYHEGYACTSIHGDRSQRDREEALHQFRSGKSPILVATAVAARGLDISNVKHVINFDLPSDIEEYVHRIGRTGRVGNLGLATSFFNERNINITKDLLDLLVEAKQEVPSWLENMAYEHHYKGSSRGRSKSSRFSGGFGARDYRQSSGASSSSFSSSRASSSRSGGGGHGSSRGFGGGGYGGFYNSDGYGGNYNSQGVDWWGN

SEQ ID NO: 32 - protein, 311 aa, Homo sapiens
MSQRDTLVHLFAGGCGGTVGAILTCPLEVVKTRLQSSSVTLYISEVQLNTMAGASVNRVVSPGPLHCLKVILEKEGPRSLFRGLGPNLVGVAPSRAIYFAAYSNCKEKLNDVFDPDSTQVHMISAAMAGFTAITATNPIWLIKTRLQLDARNRGERRMGAFECVRKVYQTDGLKGFYRGMSASYAGISETVIHFVIYESIKQKLLEYKTASTMENDEESVKEASDFVGMMLAAATSKTCATTIAYPHEVVRTRLREEGTKYRSFFQTLSLLVQEEGYGSLYRGLTTHLVRQIPNTAIMMATYELVVYLLNG

SEQ ID NO: 33 - protein, 290 aa, Homo sapiens
MSNKEGSGGFRKRKHDNFPHNQRREGKDVNSSSPVMLAFKSFQQELDARHDKYERLVKLSRDITVESKRTIFLLHRITSAPDMEDILTESEIKLDGVRQKIFQVAQELSGEDMHQFHRAITTGLQEYVEAVSFQHFIKTRSLISMDEINKQLIFTTEDNGKENKTPSSDAQDKQFGTWRLRVTPVDYLLGVADLTGELMRMCINSVGNGDIDTPFEVSQFLRQVYDGFSFIGNTGPYEVSKKLYTLKQSLAKVENACYALKVRGSEIPKHMLADVFSVKTEMIDQEEGIS

SEQ ID NO: 34 - protein, 457 aa, Homo sapiens
MRRKKSTRGDQAPGHDREAEAAVKEKEEGPEPRLTIDEAPQERLRETEGPDFPSPSAPLKASDPEEASQAQEDPGQGDQQVCAEEADPELEPASGGEQEPRPQQAQTKAASQILENGEEAPGPDPSLDRMLSSSSSVSSLNSSTLSGSQMSLSGDAEAVQVRGSVHFALHYEPGAAELRVHVIQCQGLAAARRRRSDPYVKSYLLPDKQSKRKTAVKKRNLNPVFNETLRYSVPQAELQGRVLSLSVWHRESLGRNIFLGEVEVPLDTWDWGSEPTWLPLQPRVPPSPDDLPSRGLLALSLKYVPAGSEGAGLPPSGELHFWVKEARDLLPLRAGSLDTYVQCFVLPDDSQASRQRTRVVRRSLSPVFNHTMVYDGFGPADLRQACAELSLWDHGALANRQLGGTRLSLGTGSSYGLQVPWMDSTPEEKQLWQALLEQPCEWVDGLLPLRTNLAPRT

SEQ ID NO: 35 - protein, 620 aa, Homo sapiens
MNLERLRKRVRQYLDQQQYQSALFWADKVASLSREEPQDIYWLAQCLYLTAQYHRAAHALRSRKLDKLYEACRYLAARCHYAAKEHQQALDVLDMEEPINKRLFEKYLKDESGFKDPSSDWEMSQSSIKSSICLLRGKIYDALDNRTLATYSYKEALKLDVYCFEAFDLLTSHHMLTAQEEKELLESLPLSKLCNEEQELLRFLFENKLKKYNKPSETVIPESVDGLQENLDVVVSLAERHYYNCDFKMCYKLTSVVMEKDPFHASCLPVHIGTLVELNKANELFYLSHKLVDLYPSNPVSWFAVGCYYLMVGHKNEHARRYLSKATTLEKTYGPAWIAYGHSFAVESEHDQAMAAYFTAAQLMKGCHLPMLYIGLEYGLTNNSKLAERFFSQALSIAPEDPFVMHEVGVVAFQNGEWKTAEKWFLDALEKIKAIGNEVTVDKWEPLLNNLGHVCRKLKKYAEALDYHRQALVLIPQNASTYSAIGYIHSLMGNFENAVDYFHTALGLRRDDTFSVTMLGHCIEMYIGDSEAYIGADIKDKLKCYDFDVHTMKTLKNIISPPWDFREFEVEKQTAEETGLTPLETSRKTPDSRPSLEETFEIEMNESDMMLETSMSDHST

SEQ ID NO: 36 - protein, 645 aa, Homo sapiens
MDFSFSFMQGIMGNTIQQPPQLIDSANIRQEDAFDNNSDIAEDGGQTPYEATLQQGFQYPATTEDLPPLTNGYPSSISVYETQTKYQSYNQYPNGSANGFGAVRNFSPTDYYHSEIPNTRPHEILEKPSPPQPPPPPSVPQTVIPKKTGSPEIKLKITKTIQNGRELFESSLCGDLLNEVQASEHTKSKHESRKEKRKKSNKHDSSRSEERKSHKIPKLEPEEQNRPNERVDTVSEKPREEPVLKEEAPVQPILSSVPTTEVSTGVKFQVGDLVWSKVGTYPWWPCMVSSDPQLEVHTKINTRGAREYHVQFFSNQPERAWVHEKRVREYKGHKQYEELLAEATKQASNHSEKQKIRKPRPQRERAQWDIGIAHAEKALKMTREERIEQYTFIYIDKQPEEALSQAKKSVASKTEVKKTRRPRSVLNTQPEQTNAGEVASSLSSTEIRRHSQRRHTSAEEEEPPPVKIAWKTAAARKSLPASITMHKGSLDLQKCNMSPVVKIEQVFALQNATGDGKFIDQFVYSTKGIGNKTEISVRGQDRLIISTPNQRNEKPTQSVSSPEATSGSTGSVEKKQQRRSIRTRSESEKSTEVVPKKKIKKEQVETVPQATVKTGLQKGSADRGVQGSVRFSDSSVSAAIEETVD

SEQ ID NO: 37 - protein, 297 aa, Homo sapiens
MYAQPVTNTKEVKWQKVLYERQPFPDNYVDRRFLEELRKNIHARKYQYWAVVFESSVVIQQLCSVCVFVVIWWYMDEGLLAPHWLLGTGLASSLIGYVLFDLIDGGEGRKKSGQTRWADLKSALVFITFTYGFSPVLKTLTESVSTDTIYAMSVFMLLGHLIFFDYGANAAIVSSTLSLNMAIFASVCLASRLPRSLHAFIMVTFAIQIFALWPMLQKKLKACTPRSYVGVTLLFAFSAVGGLLSISAVGAVLFALLLMSISCLCPFYLIRLQLFKENIHGPWDEAEIKEDLSRFLS

SEQ ID NO: 38 - protein, 657 aa, Homo sapiens
MRRAVCFPALCLLLNLHAAGCFSGNNDHFLAINQKKSGKPVFIYKHSQDIEKSLDIAPQKIYRHSYHSSSEAQVSKRHQIVNSAFPRPAYDPSLNLLAMDGQDLEVENLPIPAANVIVVTLQMDVNKLNITLLRIFRQGVAAALGLLPQQVHINRLIGKKNSIELFVSPINRKTGISDALPSEEVLRSLNINVLHQSLSQFGITEVSPEKNVLQGQHEADKIWSKEGFYAVVIFLSIFVIIVTCLMILYRLKERFQLSLRQDKEKNQEIHLSPITLQPALSEAKTVHSMVQPEQAPKVLNVVVDPQGRGAPEIRATTATSVCPSPFKMKPIGLQERRGSNVSLTLDMSSLGNIEPFVSIPTPREKVAMEYLQSASRILTRSQLRDVVASSHLLQSEFMEIPMNFVDPKEIDIPRHGTKNRYKTILPNPLSRVCLRPKNVTDSLSTYINANYIRGYSGKEKAFIATQGPMINTVDDFWQMVWQEDSPVIVMITKLKEKNEKCVLYWPEKRGIYGKVEVLVISVNECDNYTIRNLVLKQGSHTQHVKHYWYTSWPDHKTPDSAQPLLQLMLDVEEDRLASQGRGPVVVHCSAGIGRTGCFIATSIGCQQLKEEGVVDALSIVCQLRMDRGGMVQTSEQYEFVHHALCLYESRLSAETVQ

SEQ ID NO: 39 - protein, 887 aa, Homo sapiens
MGEAEKFHYIYSCDLDINVQLKIGSLEGKREQKSYKAVLEDPMLKFSGLYQETCSDLYVTCQVFAEGKPLALPVRTSYKAFSTRWNWNEWLKLPVKYPDLPRNAQVALTIWDVYGPGKAVPVGGTTVSLFGKYGMFRQGMHDLKVWPNVEADGSEPTKTPGRTSSTLSEDQMSRLAKLTKAHRQGHMVKVDWLDRLTFREIEMINESEKRSSNFMYLMVEFRCVKCDDKEYGIVYYEKDGDESSPILTSFELVKVPDPQMSMENLVESKHHKLARSLRSGPSDHDLKPNAATRDQLNIIVSYPPTKQLTYEEQDLVWKFRYYLTNQEKALTKFLKCVNWDLPQEAKQALELLGKWKPMDVEDSLELLSSHYTNPTVRRYAVARLRQADDEDLLMYLLQLVQALKYENFDDIKNGLEPTKKDSQSSVSENVSNSGINSAEIDSSQIITSPLPSVSSPPPASKTKEVPDGENLEQDLCTFLISRACKNSTLANYLYWYVIVECEDQDTQQRDPKTHEMYLNVMRRFSQALLKGDKSVRVMRSLLAAQQTFVDRLVHLMKAVQRESGNRKKKNERLQALLGDNEKMNLSDVELIPLPLEPQVKIRGIIPETATLFKSALMPAQLFFKTEDGGKYPVIFKHGDDLRQDQLILQIISLMDKLLRKENLDLKLTPYKVLATSTKHGFMQFIQSVPVAEVLDTEGSIQNFFRKYAPSENGPNGISAEVMDTYVKSCAGYCVITYILGVGDRHLDNLLLTKTGKLFHIDFGYILGRDPKPLPPPMKLNKEMVEGMGGTQSEQYQEFRKQCYTAFLHLRRYSNLILNLFSLMVDANIPDIALEPDKTVKKVQDKFRLDLSDEEAVHYMQSLIDESVHALFAAVVEQIHKFAQYWRK

SEQ ID NO: 40 - protein, 926 aa, Homo sapiens
MTSRFRLPAGRTYNVRASELARDRQHTEVVCNILLLDNTVQAFKVNKHDQGQVLLDVVFKHLDLTEQDYFGLQLADDSTDNPRWLDPNKPIRKQLKRGSPYSLNFRVKFFVSDPNKLQEEYTRYQYFLQIKQDILTGRLPCPSNTAALLASFAVQSELGDYDQSENLSGYLSDYSFIPNQPQDFEKEIAKLHQQHIGLSPAEAEFNYLNTARTLELYGVEFHYARDQSNNEIMIGVMSGGILIYKNRVRMNTFPWLKIVKISFKCKQFFIQLRKELHESRETLLGFNMVNYRACKNLWKACVEHHTFFRLDRPLPPQKNFFAHYFTLGSKFRYCGRTEVQSVQYGKEKANKDRVFARSPSKPLARKLMDWEVVSRNSISDDRLETQSLPSRSPPGTPNHRNSTFTQEGTRLRPSSVGHLVDHMVHTSPSEVFVNQRSPSSTQANSIVLESSPSQETPGDGKPPALPPKQSKKNSWNQIHYSHSQQDLESHINETFDIPSSPEKPTPNGGIPHDNLVLIRMKPDENGRFGFNVKGGYDQKMPVIVSRVAPGTPADLCVPRLNEGDQVVLINGRDIAEHTHDQVVLFIKASCERHSGELMLLVRPNAVYDVVEEKLENEPDFQYIPEKAPLDSVHQDDHSLRESMIQLAEGLITGTVLTQFDQLYRKKPGMTMSCAKLPQNISKNRYRDISPYDATRVILKGNEDYINANYINMEIPSSSIINQYIACQGPLPHTCTDFWQMTWEQGSSMVVMLTTQVERGRVKCHQYWPEPTGSSSYGCYQVTCHSEEGNTAYIFRKMTLFNQEKNESRPLTQIQYIAWPDHGVPDDSSDFLDFVCHVRNKRAGKEEPVVVHCSAGIGRTGVLITMETAMCLIECNQPVYPLDIVRTMRDQRAMMIQTPSQYRFVCEAILKVYEEGFVKPLTTSTNK

SEQ ID NO: 41 - protein, 289 aa, Homo sapiens
MEQPGPRAPDPSLCHHNLQPTDDPNWDSYATTMRTAFTPKTGAVPALIRQNGIRRLGYTYSLSDPILNQTQYSDEYTWKSHSKEDLIKTETSRGIKSHKSHLNEDIFLWTLPHCQQTGTLKNCLPWKIPASMKEVNKALSNQFISLTKRDFVDRSKAQKIKKSSHLSLEWKKLLPQPPDTEFRRNYQIPAKIPELQDFSFKYGCYSSLPVASQGLVPSVLHSYLRNQEHTKKQTTYQSDYDKTYPDFLMLLNSFTSSQVKEYLQSLSYKDRQIIDRFIRTHCDTNKKKK

SEQ ID NO: 42 - protein, 928 aa, Homo sapiens
MGGSASSQLDEGKCAYIRGKTEAAIKNFSPYYSRQYSVAFCNHVRTEVEQQRDLTSQFLKTKPPLAPGTILYEAELSQFSEDIKKWKERYVVVKNDYAVESYENKEAYQRGAAPKCRILPAGGKVLTSEDEYNLLSDRHFPDPLASSEKENTQPFVVLPKEFPVYLWQPFFRHGYFCFHEAADQKRFSALLSDCVRHLNHDYMKQMTFEAQAFLEAVQFFRQEKGHYGSWEMITGDEIQILSNLVMEELLPTLQTDLLPKMKGKKNDRKRTWLGLLEEAYTLVQHQVSEGLSALKEECRALTKGLEGTIRSDMDQIVNSKNYLIGKIKAMVAQPAEKSCLESVQPFLASILEELMGPVSSGFSEVRVLFEKEVNEVSQNFQTTKDSVQLKEHLDRLMNLPLHSVKMEPCYTKVNLLHERLQDLKSRFRFPHIDLVVQRTQNYMQELMENAVFTFEQLLSPHLQGEASKTAVAIEKVKLRVLKQYDYDSSTIRKKIFQEALVQITLPTVQKALASTCKPELQKYEQFIFADHTNMIHVENVYEEILHQILLDETLKVIKEAAILKKHNLFEDNMALPSESVSSLTDLKPPTGSNQASPARRASAILPGVLGSETLSNEVFQESEEEKQPEVPSSLAKGESLSLPGPSPPPDGTEQVIISRVDDPVVNPVATEDTAGLPGTCSSELEFGGTLEDEEPAQEEPEPITASGSLKALRKLLTASVEVPVDSAPVMEEDTNGESHVPQENEEEEEKEPSQAAAIHPDNCEESEVSEREAQPPCPEAHGEELGGFPEVGSPASPPASGGLTEEPLGPMEGELPGEACTLTAHEGRGGKCTEEGDASQQEGCTLGSDPICLSESQVSEEQEEMGGQSSAAQATASVNAEEIKVARIHECQWVVEDAPNPDVLLSHKDDVKEGEGGQESFPELPSEE

SEQ ID NO: 43 - protein, 301 aa, Homo sapiens
MSGLLTDPEQRAQEPRYPGFVLGLDVGSSVIRCHVYDRAARVCGSSVQKVENLYPQIGWVEIDPDVLWIQFVAVIKEAVKAAGIQMNQIVGLGISTQRATFITWNKKTGNHFHNFISWQDLRAVELVKSWNNSLLMKIFHSSCRVLHFFTRSKRLFTASLFTFTTQQTSLRLVWILQNLTEVQKAVEEENCCFGTIDTWLLYKLTKGSVYATDFSNASTTGLFDPYKMCWSGMITSLISIPLSLLPPVRDTSHNFGSVDEEIFGVPIPIVALLKVPGYDQNICYIFGKGTIEPVFYHGSTL

SEQ ID NO: 44 - protein, 295 aa, Homo sapiens
MVDSVYRTRSLGVAAEGLPDQYADGEAARVWQLYIGDTRSRTAEYKAWLLGLLRQHGCQRVLDVACGTGVDSIMLVEEGFSVTSVDASDKMLKYALKERWNRRHEPAFDKWVIEEANWMTLDKDVPQSAEGGFDAVICLGNSFAHLPDCKGDQSEHRLALKNIASMVRAGGLLVIDHRNYDHILSTGCAPPGKNIYYKSDLTKDVTTSVLIVNNKAHMVTLDYTVQVPGAGQDGSPGLSKFRLSYYPHCLASFTELLQAAFGGKCQHSVLGDFKPYKPGQTYIPCYFIHVLKRTD

SEQ ID NO: 45 - protein, 985 aa, Homo sapiens
MDRRSMGETESGDAFLDLKKPPASKCPHRYTKEELLDIKELPHSKQRPSCLSEKYDSDGVWDPEKWHASLYPASGRSSPVESLKKELDTDRPSLVRRIVDPRERVKEDDLDVVLSPQRRSFGGGCHVTAAVSSRRSGSPLEKDSDGLRLLGGRRIGSGRIISARTFEKDHRLSDKDLRDLRDRDRERDFKDKRFRREFGDSKRVFGERRRNDSYTEEEPEWFSAGPTSQSETIELTGFDDKILEEDHKGRKRTRRRTASVKEGIVECNGGVAEEDEVEVILAQEPAADQEVPRDAVLPEQSPGDFDFNEFFNLDKVPCLASMIEDVLGEGSVSASRFSRWFSNPSRSGSRSSSLGSTPHEELERLAGLEQAILSPGQNSGNYFAPIPLEDHAENKVDILEMLQKAKVDLKPLLSSLSANKEKLKESSHSGVVLSVEEVEAGLKGLKVDQQVKNSTPFMAEHLEETLSAVTNNRQLKKDGDMTAFNKLVSTMKASGTLPSQPKVSRNLESHLMSPAEIPGQPVPKNILQELLGQPVQRPASSNLLSGLMGSLEPTTSLLGQRAPSPPLSQVFQTRAASADYLRPRIPSPIGFTPGPQQLLGDPFQGMRKPMSPITAQMSQLELQQAALEGLALPHDLAVQAANFYQPGFGKPQVDRTRDGFRNRQQRVTKSPAPVHRGNSSSPAPAASITSMLSPSFTPTSVIRKMYESKEKSKEEPASGKAALGDSKEDTQKASEENLLSSSSVPSADRDSSPTTNSKLSALQRSSCSTPLSQANRYTKEQDYRPKATGRKTPTLASPVPTTPFLRPVHQVPLVPHVPMVRPAHRLHPGLVQRMLAQGVHPQHLPSLLQTGVLPPGMDLSHLQGISGPILGQPFYPLPAASHPLLNPRPGTPLHLAMVQQQLQRSVLHPPGSGSHAAAVSVQTTPQNVPSRSGLPHMHSQLEHRPSQRSSSPVGLAKWFGSDVLQQPLPSMPAKVISVDELEYRQ

SEQ ID NO: 46 - protein, 289 aa, Homo sapiens
MWRSCLRLRDGGRRLLNRPAGGPSASMSPGPTIPSPARAYAPPTERKRFYQNVSITQGEGGFEINLDHRKLKTPQAKLFTVPSEALAIAVATEWDSQQDTIKYYTMHLTTLCNTSLDNPTQRNKDQLIRAAVKFLDTDTICYRVEEPETLVELQRNEWDPIIEWAEKRYGVEISSSTSIMGPSIPAKTREVLVSHLASYNTWALQGIEFVAAQLKSMVLTLGLIDLRLTVEQAVLLSRLEEEYQIQKWGNIEWAHDYELQELRARTAAGTLFIHLCSESTTVKHKLLKE

SEQ ID NO: 47 - protein, 984 aa, Homo sapiens
MTVRRLSLLCRDLWALWLLLKAGADEIMHQDIVPLCAADIQDQLKKRFAYLSGGRGQDGSPVITFPDYPAFSEIPDKEFQNVMTYLTSIPSLQDAGIGFILVIDRRRDKWTSVKASVLRIAASFPANLQLVLVLRPTGFFQRTLSDIAFKFNRDDFKMKVPVIMLSSVPDLHGYIDKSQLTEDLGGTLDYCHSRWLCQRTAIESFALMVKQTAQMLQSFGTELAETELPNDVQSTSSVLCAHTEKKDKAKEDLRLALKEGHSVLESLRELQAEGSEPSVNQDQLDNQATVQRLLAQLNETEAAFDEFWAKHQQKLEQCLQLRHFEQGFREVKAILDAASQKIATFTDIGNSLAHVEHLLRDLASFEEKSGVAVERARALSLDGEQLIGNKHYAVDSIRPKCQELRHLCDQFSAEIARRRGLLSKSLELHRRLETSMKWCDEGIYLLASQPVDKCQSQDGAEAALQEIEKFLETGAENKIQELNAIYKEYESILNQDLMEHVRKVFQKQASMEEVFHRRQASLKKLAARQTRPVQPVAPRPEALAKSPCPSPGIRRGSENSSSEGGALRRGPYRRAKSEMSESRQGRGSAGEEEESLAILRRHVMSELLDTERAYVEELLCVLEGYAAEMDNPLMAHLLSTGLHNKKDVLFGNMEEIYHFHNRIFLRELENYTDCPELVGRCFLERMEDFQIYEKYCQNKPRSESLWRQCSDCPFFQECQRKLDHKLSLDSYLLKPVQRITKYQLLLKEMLKYSRNCEGAEDLQEALSSILGILKAVNDSMHLIAITGYDGNLGDLGKLLMQGSFSVWTDHKRGHTKVKELARFKPMQRHLFLHEKAVLFCKKREENGEGYEKAPSYSYKQSLNMAAVGITENVKGDAKKFEIWYNAREEVYIVQAPTPEIKAAWVNEIRKVLTSQLQACREASQHRALEQSQSLPLPAPTSTSPSRGNSRNIKKLEERKTDPLSLEGYVSSAPLTKPPEKGKEP

SEQ ID NO: 48 - protein, 903 aa, Homo sapiens
MATSADSPSSPLGAEDLLSDSSEPPGLNQVSSEVTSQLYASLRLSRQAEATARAQLYLPSTSPPHEGLDGFAQELSRSLSVGLEKNLKKKDGSKHIFEMESVRGQLQTMLQTSRDTAYRDPLIPGAGSERREEDSFDSDSTATLLNTRPLQDLSPSSSAQALEELFPRYTSLRPGPPLNPPDFQGLRDALDSEHTRRKHCERHIQSLQTRVLELQQQLAVAVAADRKKDTMIEQLDKTLARVVEGWNRHEAERTEVLRGLQEEHQAAELTRSKQQETVTRLEQSLSEAMEALNREQESARLQQRERETLEEERQALTLRLEAEQQRCCVLQEERDAARAGQLSEHRELETLRAALEEERQTWAQQEHQLKEHYQALQEESQAQLEREKEKSQREAQAAWETQHQLALVQSEVRRLEGELDTARRERDALQLEMSLVQARYESQRIQLESELAVQLEQRVTERLAQAQESSLRQAASLREHHRKQLQDLSGQHQQELASQLAQFKVEMAEREERQQQVAEDYELRLAREQARVCELQSGNQQLEEQRVELVERLQAMLQAHWDEANQLLSTTLPPPNPPAPPAGPSSPGPQEPEKEERRVWTMPPMAVALKPVLQQSREARDELPGAPPVLCSSSSDLSLLLGPSFQSQHSFQPLEPKPDLTSSTAGAFSALGAFHPDHRAERPFPEEDPGPDGEGLLKQGLPPAQLEGLKNFLHQLLETVPQNNENPSVDLLPPKSGPLTVPSWEEAPQVPRIPPPVHKTKVPLAMASSLFRVPEPPSSHSQGSGPSSGSPERGGDGLTFPRQLMEVSQLLRLYQARGWGALPAEDLLLYLKRLEHSGTDGRGDNVPRRNTDSRLGEIPRKEIPSQAVPRRLATAPKTEKPPARKKSGHPAPSSMRSRGGVWR

SEQ ID NO: 49 - protein, 294 aa, Homo sapiens
MASPGKPGADEAQEEEGELEGGSAGPRAAILEQAEELFLLCDKEAKGFITKHDLQGLQSDLPLTPEQLEAVFESLDQAHTGFLTAREFCLGLGMFVGVASAQGANPCRTPEETFESGGLDVQGTAGSLDEEEEEEERFHTVLEQLGVAPVLGEQRAVRTLWARLQRERPELLGSFEDVLIRASACLEEAARERDGLEQALRRRESEHEREVRALYEETEQLREQSRRPPSQNFARGERRSRLELELQIREQDLERAGLRQRERRGRRLQEHAERESQPATATGAAQGAEYTAAG

SEQ ID NO: 50 - protein, 459 aa, Homo sapiens
MYDRAPRLLKLAEGGSTEAHVGPGSYQVPFLKQQATGSNAPFLSLTARESTFTIASSIEKAVPGPGHYNVSEAQKISRSPTLTRSVDVPSIPSCGKSYGYHINDDGSIIKCFPPACDSTLGPAYYKPQFDVSNATLKYKGIHFGNSSGRQELPKKSGPGPGQYDIVQKKTSYYENVNIKRDQQQNYCSFIPRLYEIIVLQEKKKRFLPMKSITPAPGTYNEPRTALKSLKKTSGLKNIPFGQSAVRFTQDIRTEEMPGPGFYNVLNNTIIASVRNICSKKQKKSAFGSSVPRTFFSVQKEACATPGPADYQEFWHSQGVGISDELPNLTNKYAAFLSRAKRTMKVPDMVIPAPGSYDVHKSYEMSQVKHKYMPPRSLVAKRKHASFLSATPRCLEKVTDGPGPAAYNPVLRKSCPIPLFVKASKRFEESKEITPGPATYEISQEKKKGNLIGEMAADIM

SEQ ID NO: 51 - protein, 884 aa, Homo sapiens
MTTEVSPSQVSITEEEVGEEPVSTSHFSTSHLVSHTVFRCQLCKYFCSTRKGIARHYRIKHNNVRAQPEGKNNLFKCALCAYTNPIRKGLAAHYQKRHDIDAYYTHCLAASRTISDKPNKVIIPSPPKDDSPQLSEELRRAVEKKKCSLCSFQSFSKKGIVSHYMKRHPGVFPKKQHASKLGGYFTAVYADEHEKPTLMEEEERGSFEKAEVEGEAQEIEWLPFRCIKCFKLSFSTAELLCMHYTDHHSRDLKRDFIILGNGPRLQNSTYQCKHCDSKLQSTAELTSHLNIHNEEFQKRAKRQERRKQLLSKQKYADGAFADFKQERPFGHLEEVPKIKERKVVGYKCKFCVEVHPTLRAICNHLRKHVQYGNVPAVSAAVKGLRSHERSHLALAMFTREDKYSCQYCSFVSAFRHNLDRHMQTHHGHHKPFRCKLCSFKSSYNSRLKTHILKAHAGEHAYKCSWCSFSTMTISQLKEHSLKVHGKALTLPRPRIVSLLSSHSHHSSQKATPAEEVEDSNDSSYSEPPDVQQQLNHYQSAALARNNSRVSPVPLSGAAAGTEQKTEAVLHCEFCEFSSGYIQSIRRHYRDKHGGKKLFKCKDCSFYTGFKSAFTMHVEAGHSAVPEEGPKDLRCPLCLYHTKYKRNMIDHIVLHREERVVPIEVCRSKLSKYLQGVVFRCDKCTFTCSSDESLQQHIEKHNELKPYKCQLCYYETKHTEELDSHLRDEHKVSRNFELVGRVNLDQLEQMKEKMESSSSDDEDKEEEMNSKAEDRELMRFSDHGAALNTEKRFPCEFCGRAFSQGSEWERHVLRHGMALNDTKQVSREEIHPKEIMENSVKMPSIEEKEDDEAIGIDFSLKNETVAICVVTADKSLLENAEAKKE

SEQ ID NO: 52 - protein, 318 aa, Homo sapiens
MNRFGTRLVGATATSSPPPKARSNENLDKIDMSLDDIIKLNRKEGKKQNFPRLNRRLLQQSGAQQFRMRVRWGIQQNSGFGKTSLNRRGRVMPGKRRPNGVITGLAARKTTGIRKGISPMNRPPLSDKNIEQYFPVLKRKANLLRQNEGQRKPVAVLKRPSQLSRKNNIPANFTRSGNKLNHQKDTRQATFLFRRGLKVQAQLNTEQLLDDVVAKRTRQWRTSTTNGGILTVSIDNPGAVQCPVTQKPRLTRTAVPSFLTKREQSDVKKVPKGVPLQFDINSVGKQTGMTLNERFGILKEQRATLTYNKGGSRFVTVG

SEQ ID NO: 53 - protein, 1017 aa, Homo sapiens
MKLLKPTWVNHNGKPIFSVDIHPDGTKFATGGQGQDSGKVVIWNMSPVLQEDDEKDENIPKMLCQMDNHLACVNCVRWSNSGMYLASGGDDKLIMVWKRATYIGPSTVFGSSGKLANVEQWRCVSILRNHSGDVMDVAWSPHDAWLASCSVDNTVVIWNAVKFPEILATLRGHSGLVKGLTWDPVGKYIASQADDRSLKVWRTLDWQLETSITKPFDECGGTTHVLRLSWSPDGHYLVSAHAMNNSGPTAQIIEREGWKTNMDFVGHRKAVTVVKFNPKIFKKKQKNGSSAKPSCPYCCCAVGSKDRSLSVWLTCLKRPLVVIHELFDKSIMDISWTLNGLGILVCSMDGSVAFLDFSQDELGDPLSEEEKSRIHQSTYGKSLAIMTEAQLSTAVIENPEMLKYQRRQQQQQLDQKSAATREMGSATSVAGVVNGESLEDIRKNLLKKQVETRTADGRRRITPLCIAQLDTGDFSTAFFNSIPLSGSLAGTMLSSHSSPQLLPLDSSTPNSFGASKPCTEPVVAASARPAGDSVNKDSMNATSTPAALSPSVLTTPSKIEPMKAFDSRFTERSKATPGAPALTSMTPTAVERLKEQNLVKELRPRDLLESSSDSDEKVPLAKASSLSKRKLELEVETVEKKKKGRPRKDSRLMPVSLSVQSPAALTAEKEAMCLSAPALALKLPIPSPQRAFTLQVSSDPSMYIEVENEVTVVGGVKLSRLKCNREGKEWETVLTSRILTAAGSCDVVCVACEKRMLSVFSTCGRRLLSPILLPSPISTLHCTGSYVMALTAAATLSVWDVHRQVVVVKEESLHSILAGSDMTVSQILLTQHGIPVMNLSDGKAYCFNPSLSTWNLVSDKQDSLAQCADFRSSLPSQDAMLCSGPLAIIQGRTSNSGRQAARLFSVPHVVQQETTLAYLENQVAAALTLQSSHEYRHWLLVYARYLVNEGFEYRLREICKDLLGPVHYSTGSQWESTVVGLRKRELLKELLPVIGQNLRFQRLFTECQEQLDILRDK

SEQ ID NO: 54 - protein, 886 aa, Homo sapiens [sequence omitted]

SEQ ID NO: 55 - protein, 963 aa, Homo sapiens
MAVFPWHSRNRNYKAEFASCRLEAVPLEFGDYHPLKPITVTESKTKKVNRKGSTSSTSSSSSSSVVDPLSSVLDGTDPLSMFAATADPAALAAAMDSSRRKRDRDDNSVVGSDFEPWTNKRGEILARYTTTEKLSINLFMGSEKGKAGTATLAMSEKVRTRLEELDDFEEGSQKELLNLTQQDYVNRIEELNQSLKDAWASDQKVKALKIVIQCSKLLSDTSVIQFYPSKFVLITDILDTFGKLVYERIFSMCVDSRSVLPDHFSPENANDTAKETCLNWFFKIASIRELIPRFYVEASILKCNKFLSKTGISECLPRLTCMIRGIGDPLVSVYARAYLCRVGMEVAPHLKETLNKNFFDFLLTFKQIHGDTVQNQLVVQGVELPSYLPLYPPAMDWIFQCISYHAPEALLTEMMERCKKLGNNALLLNSVMSAFRAEFIATRSMDFIGMIKECDESGFPKHLLFRSLGLNLALADPPESDRLQILNEAWKVITKLKNPQDYINCAEVWVEYTCKHFTKREVNTVLADVIKHMTPDRAFEDSYPQLQLIIKKVIAHFHDFSVLFSVEKFLPFLDMFQKESVRVEVCKCIMDAFIKHQQEPTKDPVILNALLHVCKTMHDSVNALTLEDEKRMLSYLINGFIKMVSFGRDFEQQLSFYVESRSMFCNLEPVLVQLIHSVNRLAMETRKVMKGNHSRKTAAFVRACVAYCFITIPSLAGIFTRLNLYLHSGQVALANQCLSQADAFFKAAISLVPEVPKMINIDGKMRPSESFLLEFLCNFFSTLLIVPDHPEHGVLFLVRELLNVIQDYTWEDNSDEKIRIYTCVLHLLSAMSQETYLYHIDKVDSNDSLYGGDSKFLAENNKLCETVMAQILEHLKTLAKDEALKRQSSLGLSFFNSILAHGDLRNNKLNQLSVNLWHLAQRHGCADTRTMVKTLEYIKKQSKQPDMTHLTELALRLPLQTRT

SEQ ID NO: 56 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLNKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVPPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKAERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 57 - protein, 943 aa, Homo sapiens
MGPRKKSVKTCIMNNEIPEEMTADETKDYMNQLSHEVLCHIFRYLPLQDIMCMECLSRKLKEAVTLYLRVVRVVDLCAGRWWEYMPSGFTDASFLTLLKKMPDVEQLYGLHPRYLERRRVRGHEAFSIPGVLEALQACPNLVGVETSHLELVESIWTYMPHVHILGKFRNRNGAFPIPPENKLKIPIGAKIQTLHLVGVNVPEIPCIPMLRHLYMKWVRLTKPQPFKDFLCISLRTFVMRNCAGPTNSLKYVPLVTGLASARNLEHLEMVRVPFLGGLIQHVVEDSWRSGGFRNLHTIVLGACKNALEVDLGYLIITAARRLHEVRIQPSLTKDGVFSALKMAELEFPQFETLHLGYVDEFLLQSRMANADLVKYGLADVVENPGIITDIGMKAVNEVFSCIKYLAIYNCPHLHNPYNWISDHSRWTRLVDINLVRCHALKLDSFGQFIELLPSLEFISLDQMFREPPKGCARVGLSAGTGIGVSSALVSNQNSNNDDNNAQNNNANIHDNNHHHPDDSDEENDFRQDLQPGEQQFAADALNEMEDIVQEDGEVVAESGNNTPAHSQAIIPVDVDEEQAGPSGLQRVVKPTSITVHDSESDDEEDSLELQEVWIPKNGTRRYSEREEKTGESVQSRELSEVAKTKPRHAMKRKRTADKSTSTSDPVIEDDHVQVLVLKSKNLVGVTMTNCGITDLVLKDCPKMMFIHATRCRVLKHLKVENAPIVNRFDYAQCKKLNMDQVLDQILRMPPERNRIIYLRPMQQVDTLTLEQKLFSGPYPYHICIIHEFSNPPNVRNKVRIRSWMDTIANINQELIKYEFFPEATRSEEDLKKYPKYPWGREIYTLEGVVDGAPYSMISDFPWLRSLRAAEPNSFARYDFEDDEESTIYAPRRKGQLSADICMETIGEEISEMRQMKKGVFQRVVAIFIHYCDVNGEPVEDDYI

SEQ ID NO: 58 - protein, 920 aa, Homo sapiens
MTAEETVNVKEVEIIKLILDFLNSKKLHISMLALEKESGVINGLFSDDMLFLRQLILDGQWDEVLQFIQPLECMEKFDKKRFRYIILKQKFLEALCVNNAMSAEDEPQHLEFTMQEAVQCLHALEEYCPSKDDYSKLCLLLTLPRLTNHAEFKDWNPSTARVHCFEEACVMVAEFIPADRKLSEAGFKASNNRLFQLVMKGLLYECCVEFCQSKATGEEITESEVLLGIDLLCGNGCDDLDLSLLSWLQNLPSSVFSCAFEQKMLNIHVDKLLKPTKAAYADLLTPLISKLSPYPSSPMRRPQSADAYMTRSLNPALDGLTCGLTSHDKRISDLGNKTSPMSHSFANFHYPGVQNLSRSLMLENTECHSIYEESPERSDTPVDAQRPIGSEILGQSSVSEKEPANGAQNPGPAKQEKNELRDSTEQFQEYYRQRLRYQQHLEQKEQQRQIYQQMLLEGGVNQEDGPDQQQNLTEQFLNRSIQKLGELNIGMDGLGNEVSALNQQCNGSKGNGSNGSSVTSFTTPPQDSSQRLTHDASNIHTSTPRNPGSTNHIPFLEESPCGSQISSEHSVIKPPLGDSPGSLSRSKGEEDDKSKKQFVCINILEDTQAVRAVAFHPAGGLYAVGSNSKTLRVCAYPDVIDPSAHETPKQPVVRFKRNKHHKGSIYCVAWSPCGQLLATGSNDKYVKVLPFNAETCNATGPDLEFSMHDGTIRDLAFMEGPESGGAILISAGAGDCNIYTTDCQRGQGLHALSGHTGHILALYTWSGWMIASGSQDKTVGFWDLRVPSCARVVGTTFHGTGSAVASVAVDPSGRLLATGQEDSSCMLYDIRGGRMVQSYHPHSSDVRSVRFSPGAHYLLTGSYDMKIKVTDLQGDLTKQLPIMVVGEHKDKVIQCRWHTQDLSFLSSSADRTVTLWTYNG

SEQ ID NO: 59 - protein, 295 aa, Homo sapiens
MSGALDVLQMKEEDVLKFLAAGTHLGGTNLDFQMEQYIYKRKSDGIYIINLKRTWEKLLLAARAIVAIENPADVSVISSRNTGQRAVLKFAAATGATPIAGRFTPGTFTNQIQATFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTATQPEVADWSEGVQVPSVPIQQFPTEDWSAQPATEDWSAAPTAQATEWVGATTDWS

SEQ ID NO: 60 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 61 - protein, 445 aa, Homo sapiens
MSKLPRELTRDLERSLPAVASLGSSLSHSQSLSSHLLPPPEKRRAISDVRRTFCLFVTFDLLFISLLWIIELNTNTGIRKNLEQEIIQYNFKTSFFDIFVLAFFRFSGLLLGYAVLRLRHWWVIAVTTLVSSAFLIVKVILSELLSKGAFGYLLPIVSFVLAWLETWFLDFKVLPQEAEEERWYLAAQVAVARGPLLFSGALSEGQFYSPPESFAASDNESDEEVAGKKSFSAQEREYIRQGKEATAVVDQILAQEENWKFEKNNEYGDTVYTIEVPFHGKTFILKTFLPCPAELVYQEVILQPERMVLWNKTVTACQILQRVEDNTLISYDVSAGAAGGVVSPRDFVNVRRIERRRDRYLSSGIATSHSAKPPTHKYVRGENGPGGFIVLKSASNPRVCTFVWILNTDLKGRLPRYLIHQSLAATMFEFAFHLRQRISELGARA

SEQ ID NO: 62 - protein, 946 aa, Homo sapiens [sequence omitted]

SEQ ID NO: 63 - protein, 948 aa, Homo sapiens
MEEKEMLRRQIRLLQGLIDDYKTLHGNAPAPGTPAASGWQPPTYHSGRAFSARYPRPSRRGYSSHHGPSWRKKYSLVNRPPGPSDPPADHAVRPLHGARGGRPPVPQQHVLERQVQLSQGQNVVIKVKPPSKSGSASASGAQRGSLEEFEETPWSDQRPREGEGKPPRGQLQPSRPTRARGTCSVEDPLLVCQKEPGKPRMVKSVGSVGDSPREPRRTVSESVIAVKASFASSALPPRTGVALGRKLGSHSVASCAPQLLGDRRVDAGHTDQPVPSGSVGGPARPASGPRQAREASLVVTCRTNKFRKNNYKWVAASSKSPRVARRALSPRVAAENVCKASAGMANKVEKPQLIADPEPKPRKPATSSKPGSAPSKYKWKASSPSASSSSSFRWQSEASSKDHASQLSPVLSRSPSGDRPAVGHSGLKPLSGETPLSAYKVKSRTKIIRRRGSTSLPGDKKSGTSPAATAKSHLSLRRRQALRGKSSPVLKKTPNKGLVQVTTHRLCRLPPSRAHLPTKEASSLHAVRTAPTSKVIKTRYRIVKKTPASPLSAPPFPLSLPSWRARRLSLSGSLVLNHLRPVASGGGKAQPGSPWWRSKGYRCIGGVLYKVSANKLSKTSGQPSDAGSRPLLRTGWLDPAGSCSRSLASRAVQRSLAIIRQARQRREKRKEYCMYYNRFGRCNRGERCPYIHDPEKVAVCTRFVRGTCKKTDGTCPFSHHVSKEKMRVCSYFLKGICSNSNCPYSHVYVSRKAEVCSDFLKGYCPLGAKCKKKHTLLCPDFARRGACPRGAQCQLLHRTQKRHSRRAATSPAPGPSDATARSRVSASHGPRKPSASQRPTRQTPSSAALTAAAVAAPPHCPGGSASPSSSKASSSSSSSSSPPASLDHEAPSLQEAALAAACSNRLCKLPSFISLQSSPSPGAQPRVRAPRAPLTKDSGKPLHIKPRL

SEQ ID NO: 64 - protein, 298 aa, Homo sapiens
MWGACKVKVHDSLATISITLRRYLRLGATMAKSKFEYVRDFEADDTCLAHCWVVVRLDGRNFHRFAEKHNFAKPNDSRALQLMTKCAQTVMEELEDIVIAYGQSDEYSFVFKRKTNWFKRRASKFMTHVASQFASSYVFYWRDYFEDQPLLYPPGFDGRVVVYPSNQTLKDYLSWRQADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKPVPLHCDIIGDAFWKEHPEILDEDS

SEQ ID NO: 65 - protein, 313 aa, Homo sapiens
MCTALSPKVRSGPGLSDMHQYSQWLASRHEANLLPMKEDLALWLTNLLGKEITAETFMEKLDNGALLCQLAETMQEKFKESMDANKPTKNLPLKKIPCKTSAPSGSFFARDNTANFLSWCRDLGVDETCLFESEGLVLHKQPREVCLCLLELGRIAARYGVEPPGLIKLEKEIEQEETLSAPSPSPSPSSKSSGKKSTGNLLDDAVKRISEDPPCKCPNKFCVERLSQGRYRVGEKILFIRMLHNKHVMVRVGGGWETFAGYLLKHDPCRMLQISRVDGKTSPIQSKSPTLKDMNPDNYLVVSASYKAKKEIK

SEQ ID NO: 66 - protein, 314 aa, Homo sapiens
MAKNNLTRVTEFILMGFMDHPKLEIPLFLVFLSFYLVTLLGNVGMIMLIQVDVKLYTPMYFFLSHLSLLDACYTSVITPQILATLATGKTVISYGHCAAQFFLFTICAGTECFLLAVMAYDRYAAIRNPLLYTVAMNPRLCWSLVVGAYVCGVSGAILRTTCTFTLSFCKDNQINFFFCDLPPLLKLACSDTANIEIVIIFFGNFVILANASVILISYLLIIKTILKVKSSGGRAKTFSTCASHITAVALFFGALIFMYLQSGSGKSLEEDKVVSVFYTVVIPMLNPLIYSLRNKDVKDAFRKVARRLQVSLSM

SEQ ID NO: 67 - protein, 644 aa, Homo sapiens
MDDIADRMRMDAGEVTLVNHNSVFKTHLLPQTGFPEDQLSLSDQQILSSRQGHLDRSFTCSTRSAAYNPSYYSDNPSSDSFLGSGDLRTFGQSANGQWRNSTPSSSSSLQKSRNSRSLYLETRKTSSGLSNSFAGKSNHHCHVSAYEKSFPIKPVPSPSWSGSCRRSLLSPKKTQRRHVSTAEETVQEEEREVYRQLLQMVTGKQFTIAKPTTHFPLHLSRCLSSSKNTLKDSLFKNGNSCASQIIGSDTSSSGSASILTNQEQLSHSVYSLSSYTPDVAFGSKDSGTLHHPHHHHSVPHQPDNLAASNTQSEGSDSVILLKVKDSQTPTPSSTFFQAELWIKELTSVYGSRARERLRQIEEQKALALQLQNQRLQEREHSVHDSVELHLRVPLEKEIPVTVVQETQKKGHKLTDSEDEFPEITEEMEKEIKNVFRNGNQDEVLSEAFRLTITRKDIQTLNHLNWLNDEIINFYMNMLMERSKEKGLPSVHAFNTFFFTKLKTAGYQAVKRWTKKVDVFSVDILLVPIHLGVHWCLAVVDFRKKNITYYDSMGGINNEACRILLQYLKQESIDKKRKEFDTNGWQLFSKKSQEIPQQMNGSDCGMFACKYADCITKDRPINFTQQHMPYFRKRMVWEILHRKLL

SEQ ID NO: 68 - protein, 622 aa, Homo sapiens
MRLLRRRHMPLRLAMVGCAFVLFLFLLHRDVSSREEATEKPWLKSLVSRKDHVLDLMLEAMNNLRDSMPKLQIRAPEAQQTLFSINQSCLPGFYTPAELKPFWERPPQDPNAPGADGKAFQKSKWTPLETQEKEEGYKKHCFNAFASDRISLQRSLGPDTRPPECVDQKFRRCPPLATTSVIIVFHNEAWSTLLRTVYSVLHTTPAILLKEIILVDDASTEEHLKEKLEQYVKQLQVVRVVRQEERKGLITARLLGASVAQAEVLTFLDAHCECFHGWLEPLLARIAEDKTVVVSPDIVTIDLNTFEFAKPVQRGRVHSRGNFDWSLTFGWETLPPHEKQRRKDETYPIKSPTFAGGLFSISKSYFEHIGTYDNQMEIWGGENVEMSFRVWQCGGQLEIIPCSVVGHVFRTKSPHTFPKGTSVIARNQVRLAEVWMDSYKKIFYRRNLQAAKMAQEKSFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 69 - protein, 619 aa, Homo sapiens
MYGFVNHALELLVIRNYGPEVWEDIKKEAQLDEEGQFLVRIIYDDSKTYDLVAAASKVLNLNAGEILQMFGKMFFVFCQESGYDTILRVLGSNVREFLQNLDALHDHLATIYPGMRAPSFRCTDAEKGKGLILHYYSEREGLQDIVIGIIKTVAQQIHGTEIDMKVIQQRNEECDHTQFLIEEKESKEEDFYEDLDRFEENGTQESRISPYTFCKAFPFHIIFDRDLVVTQCGNAIYRVLPQLQPGNCSLLSVFSLVRPHIDISFHGILSHINTVFVLRSKEGLLDVEKLECEDELTGTEISCLRLKGQMIYLPEADSILFLCSPSVMNLDDLTRRGLYLSDIPLHDATRDLVLLGEQFREEYKLTQELEILTDRLQLTLRALEDEKKKTDTLLYSVLPPSVANELRHKRPVPAKRYDNVTILFSGIVGFNAFCSKHASGEGAMKIVNLLNDLYTRFDTLTDSRKNPFVYKVETVGDKYMTVSGLPEPCIHHARSICHLALDMMEIAGQVQVDGESVQITIGIHTGEVVTGVIGQRMPRYCLFGNTVNLTSRTETTGEKGKINVSEYTYRCLMSPENSDPQFHLEHRGPVSMKGKKEPMQVWFLSRKNTGTEETKQDDD

SEQ ID NO: 70 - protein, 997 aa, Homo sapiens
MDDQPGEKELVKRSQLDGEGDGPLSNQLSASSTINPVPLVGLQKPEMSLPVKPGQGDSEASSPFTPVADEDSVVFSKLTYLGCASVNAPRSEVEALRMMSILRSQCQISLDVTLSVPNVSEGIVRLLDPQTNTEIANYPIYKILFCVRGHDGTPESDCFAFTESHYNAELFRIHVFRCEIQEAVSRILYSFATAFRRSAKQTPLSATAAPQTPDSDIFTFSVSLEIKEDDGKGYFSAVPKDKDRQCFKLRQGIDKKIVIYVQQTTNKELAIERCFGLLLSPGKDVRNSDMHLLDLESMGKSSDGKSYVITGSWNPKSPHFQVVNEETPKDKVLFMTTAVDLVITEVQEPVRFLLETKVRVCSPNERLFWPFSKRSTTENFFLKLKQIKQRERKNNTDTLYEVVCLESESERERRKTTASPSVRLPQSGSQSSVIPSPPEDDEEEDNDEPLLSGSGDVSKECAEKILETWGELLSKWHLNLNVRPKQLSSLVRNGVPEALRGEVWQLLAGCHNNDHLVEKYRILITKESPQDSAITRDINRTFPAHDYFKDTGGDGQDSLYKICKAYSVYDEEIGYCQGQSFLAAVLLLHMPEEQAFSVLVKIMFDYGLRELFKQNFEDLHCKFYQLERLMQEYIPDLYNHFLDISLEAHMYASQWFLTLFTAKFPLYMVFHIIDLLLCEGISVIFNVALGLLKTSKDDLLLTDFEGALKFFRVQLPKRYRSEENAKKLMELACNMKISQKKLKKYEKEYHTMREQQAQQEDPIERFERENRRLQEANMRLEQENDDLAHELVTSKIALRKDLDNAEEKADALNKELLMTKQKLIDAEEEKRRLEEESAQLKEMCRRELDKAESEIKKNSSIIGDYKQICSQLSERLEKQQTANKVEIEKIRQKVDDCERCREFFNKEGRVKGISSTKEVLDEDTDEEKETLKNQLREMELELAQTKLQLVEAECKIQDLEHHLGLALNEVQAAKKTWFNRTLSSIKTATGVQGKETC

SEQ ID NO: 71 - protein, 643 aa, Homo sapiens
METSSSCESLGSQPAAARPPSVDSLSSASTSHSENSVHTKSASVVSSDSISTSADNFSPDLRVLRESNKLAEMEEPPLLPGENIKDMAKDVTYICPFTGAVRGTLTVTNYRLYFKSMERDPPFVLDASLGVINRVEKIGGASSRGENSYGLETVCKDIRNLRFAHKPEGRTRRSIFENLMKYAFPVSNNLPLFAFEYKEVFPENGWKLYDPLLEYRRQGIPNESWRITKINERYELCDTYPALLVVPANIPDEELKRVASFRSRGRIPVLSWIHPESQATITRCSQPMVGVSGKRSKEDEKYLQAIMDSNAQSHKIFIFDARPSVNAVANKAKGGGYESEDAYQNAELVFLDIHNIHVMRESLRKLKEIVYPNIEETHWLSNLESTHWLEHIKLILAGALRIADKVESGKTSVVVHCSDGWDRTAQLTSLAMLMLDGYYRTIRGFEVLVEKEWLSFGHRFQLRVGHGDKNHADADRSPVFLQFIDCVWQMTRQFPTAFEFNEYFLITILDHLYSCLFGTFLCNSEQQRGKENLPKRTVSLWSYINSQLEDFTNPLYGSYSNHVLYPVASMRHLELWVGYYIRWNPRMKPQEPIHNRYKELLAKRAELQKKVEELQREISNRSTSSSERASSPAQCVTPVQTVV

SEQ ID NO: 72 - protein, 454 aa, Homo sapiens
MPEFLEDPSVLTKDKLKSELVANNVTLPAGEQRKDVYVQLYLQHLTARNRPPLPAGTNSKGPPDFSSDEEREPTPVLGSGAAAAGRSRAAVGRKATKKTDKPRQEDKDDLDVTELTNEDLLDQLVKYGVNPGPIVGTTRKLYEKKLLKLREQGTESRSSTPLPTISSSAENTRQNGSNDSDRYSDNEEDSKIELKLEKREPLKGRAKTPVTLKQRRVEHNQSYSQAGITETEWTSGSSKGGPLQALTRESTRGSRRTPRKRVETSEHFRIDGPVISESTPIAETIMASSNESLVVNRVTGNFKHASPILPITEFSDIPRRAPKKPLTRAEVGEKTEERRVERDILKEMFPYEASTPTGISASCRRPIKGAAGRPLELSDFRMEESFSSKYVPKYVPLADVKSEKTKKGRSIPVWIKILLFVVVAVFLFLVYQAMETNQVNPFSNFLHVDPRKSN

SEQ ID NO: 73 - protein, 693 aa, Homo sapiens
MATSGANGPGSATASASNPRKFSEKIALQKQRQAEETAAFEEVMMDIGSTRLQAQKLRLAYTRSSHYGGSLPNVNQIGSGLAEFQSPLHSPLDSSRSTRHHGLVERVQRDPRRMVSPLRRYTRHIDSSPYSPAYLSPPPESSWRRTMAWGNFPAEKGQLFRLPSALNRTSSDSALHTSVMNPSPQDTYPGPTPPSILPSRRGGILDGEMDPKVPAIEENLLDDKHLLKPWDAKKLSSSSSRPRSCEVPGINIFPSPDQPANVPVLPPAMNTGGSLPDLTNLHFPPPLPTPLDPEETAYPSLSGGNSTSNLTHTMTHLGISRGMGLGPGYDAPGLHSPLSHPSLQSSLSNPNLQASLSSPQPQLQGSHSHPSLPASSLARHVLPTTSLGHPSLSAPALSSSSSSSSTSSPVLGAPSYPASTPGASPHHRRVPLSPLSLLAGPADARRSQQQLPKQFSPTMSPTLSSITQGVPLDTSKLSTDQRLPPYPYSSPSLVLPTQPHTPKSLQQPGLPSQSCSVQSSGGQPPGRQSHYGTPYPPGPSGHGQQSYHRPMSDFNLGNLEQFSMESPSASLVLDPPGFSEGPGFLGGEGPMGGPQDPHTFNHQNLTHCSRHGSGPNIILTGDSSPGFSKEIAAALAGVPGFEVSAAGLELGLGLEDELRMEPLGLEGLNMLSDPCALLPDPAVEESFRSDRLQ

SEQ ID NO: 74 - protein, 669 aa, Homo sapiens
MAIVQTLPVPLEPAPEAATAPQAPVMGSVSSLISGRPCPGGPAPPRHHGPPGPTFFRQQDGLLRGGYEAQEPLCPAVPPRKAVPVTSFTYINEDFRTESPPSPSSDVEDAREQRAHNAHLRGPPPKLIPVSGKLEKNMEKILIRPTAFKPVLPKPRGAPSLPSFMGPRATGLSGSQGSLTQLFGGPASSSSSSSSSSAADKPLAFSGWASGCPSGTLSDSGRNSLSSLPTYSTGGAEPTTSSPGGHLPSHGSGRGALPGPARGVPTGPSHSDSGRSSSSKSTGSLGGRLAGGLLGSGTRASPDSSSCGERSPPPPPPPPSDEALLHCVLEGKLRDREAELQQLRDSLDENEATMCQAYEERQRHWQREREALREDCAAQAQRAQRAQQLLQLQVFQLQQEKRQLQDDFAQLLQEREQLERRCATLEREQRELGPRLEETKWEVCQKSGEISLLKQQLKESQAELVQKGSELVALRVALREARATLRVSEGRARGLQEAARARELELEACSQELQRHRQEAEQLREKAGQLDAEAAGLREPPVPPATADPFLLAESDEAKVQRAAAGVGGSLRAQVERLRVELQRERRRGEEQRDSFEGERLAWQAEKEQVIRYQKQLQHNYIQMYRRNRQLEQELQQLSLELEARELADLGLAEQAPCICLEEITATEI

SEQ ID NO: 75 - protein, 442 aa, Homo sapiens
MGLAMEHGGSYARAGGSSRGCWYYLRYFFLFVSLIQFLIILGLVLFMVYGNVHVSTESNLQATERRAEGLYSQLLGLTASQSNLTKELNFTTRAKDAIMQMWLNARRDLDRINASFRQCQGDRVIYTNNQRYMAAIILSEKQCRDQFKDMNKSCDALLFMLNQKVKTLEVEIAKEKTICTKDKESVLLNKRVAEEQLVECVKTRELQHQERQLAKEQLQKVQALCLPLDKDKFEMDLRNLWRDSIIPRSLDNLGYNLYHPLGSELASIRRACDHMPSLMSSKVEELARSLRADIERVARENSDLQRQKLEAQQGLRASQEAKQKVEKEAQAREAKLQAECSRQTQLALEEKAVLRKERDNLAKELEEKKREAEQLRMELAIRNSALDTCIKTKSQPMMPVSRPMGPVPNPQPIDPASLEEFKRKILESQRPPAGIPVAPSSG

SEQ ID NO: 76 - protein, 979 aa, Homo sapiens
MVYRDDYFIRHSISVSAVIVRAWITHKYSGRDWNVKWEENLLHAVAKNYTLLQTIPPFERPFKDHQVCLEWNMGYIWNLRANRIPQCPLENDVVALLGFPYASSGENTGIVKKFPRFRNRELEATRRQRMDYPVFTVSLWLYLLHYCKANLCGILYFVDSNEMYGTPSVFLTEEGYLHIQMHLVKGEDLAVKTKFIIPLKEWFRLDISFNGGQIVVTTSIGQDLKSYHNQTISFREDFHYNDTAGYFIIGGSRYVAGIEGFFGPLKYYRLRSLHPAQIFNPLLEKQLAEQIKLYYERCAEVQEIVSVYASAAKHGGERQEACHLHNSYLDLQRRYGRPSMCRAFPWEKELKDKHPSLFQALLEMDLLTVPRNQNESVSEIGGKIFEKAVKRLSSIDGLHQISSIVPFLTDSSCCGYHKASYYLAVFYETGLNVPRDQLQGMLYSLVGGQGSERLSSMNLGYKHYQGIDNYPLDWELSYAYYSNIATKTPLDQHTLQGDQAYVETIRLKDDEILKVQTKEDGDVFMWLKHEATRGNAAAQQRLAQMLFWGQQGVAKNPEAAIEWYAKGALETEDPALIYDYAIVLFKGQGVKKNRRLALELMKKAASKGLHQAVNGLGWYYHKFKKNYAKAAKYWLKAEEMGNPDASYNLGVLHLDGIFPGVPGRNQTLAGEIFHKAAQGGHMEGTLWCSLYYITGNLETFPRDPEKAVVWAKHVAEKNGYLGHVIRKGLNAYLEGSWHEALLYYVLAAETGIEVSQTNLAHICEERPDLARRYLGVNCVWRYYNFSVFQIDAPSFAYLKMGDLYYYGHQNQSQDLELSVQMYAQAALDGDSQGFFNLALLIEEGTIIPHHILDFLEIDSTLHSNNISILQELYERCWSHSNEESFSPCSLAWLYLHLRLLWGAILHSALIYFLGTFLLSILIAWTVQYFQSVSASDPPPRPSQASPDTATSTASPAVTPAADASDQDQPTVTNNPEPRG

SEQ ID NO: 77 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGAWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 78 - protein, 441 aa, Homo sapiens
MLSSVMAPLWACILVAAGILATDTHHPQDSALYHLSKQLLQKYHKEVRPVYNWTKATTVYLDLFVHAILDVDAENQILKTSVWYQEVWNDEFLSWNSSMFDEIREISLPLSAIWAPDIIINEFVDIERYPDLPYVYVNSSGTIENYKPIQVVSACSLETYAFPFDVQNCSLTFKSILHTVEDVDLAFLRSPEDIQHDKKAFLNDSEWELLSVSSTYSILQSSAGGFAQIQFNVVMRRHPLVYVVSLLIPSIFLMLVDLGSFYLPPNCRARIVFKTSVLVGYTVFRVNMSNQVPRSVGSTPLIGHFFTICMAFLVLSLAKSIVLVKFLHDEQRGGQEQPFLCLRGDTDADRPRVEPRAQRAVVTESSLYGEHLAQPGTLKEVWSQLQSISNYLQTQDQTDQQEAEWLVLLSRFDRLLFQSYLFMLGIYTITLCSLWALWGGV

SEQ ID NO: 79 - protein, 320 aa, Homo sapiens
MADEKTFRIGFIVLGLFLLALGTFLMSHDRPQVYGTFYAMGSVMVIGGIIWSMCQCYPKITFVPADSDFQGILSPKAMGLLENGLAAEMKSPSPQPPYVRLWEEAAYDQSLPDFSHIQMKVMSYSEDHRSLLAPEMGQPKLGTSDGGEGGPGDVQAWMEAAVVIHKGSDESEGERRLTQSWPGPLACPQGPAPLASFQDDLDMDSSEGSSPNASPHDREEACSPQQEPQGCRCPLDRFQDFALIDAPTLEDEPQEGQQWEIALPNNWQRYPRTKVEEKEASDTGGEEPEKEEEDLYYGLPDGAGDLLPDKELGFEPDTQG

SEQ ID NO: 80 - protein, 1036 aa, Homo sapiens
MYLVAGDRGLAGCGHLLVSLLGLLLLLARSGTRALVCLPCDESKCEEPRNCPGSIVQGVCGCCYTCASQRNESCGGTFGIYGTCDRGLRCVIRPPLNGDSLTEYEAGVCEDENWTDDQLLGFKPCNENLIAGCNIINGKCECNTIRTCSNPFEFPSQDMCLSALKRIEEEKPDCSKARCEVQFSPRCPEDSVLIEGYAPPGECCPLPSRCVCNPAGCLRKVCQPGNLNILVSKASGKPGECCDLYECKPVFGVDCRTVECPPVQQTACPPDSYETQVRLTADGCCTLPTRCECLSGLCGFPVCEVGSTPRIVSRGDGTPGKCCDVFECVNDTKPACVFNNVEYYDGDMFRMDNCRFCRCQGGVAICFTAQCGEINCERYYVPEGECCPVCEDPVYPFNNPAGCYANGLILAHGDRWREDDCTFCQCVNGERHCVATVCGQTCTNPVKVPGECCPVCEEPTIITVDPPACGELSNCTLTGKDCINGFKRDHNGCRTCQCINTEELCSERKQGCTLNCPFGFLTDAQNCEICECRPRPKKCRPIICDKYCPLGLLKNKHGCDICRCKKCPELSCSKICPLGFQQDSHGCLICKCREASASAGPPILSGTCLTVDGHHHKNEESWHDGCRECYCLNGREMCALITCPVPACGNPTIHPGQCCPSCADDFVVQKPELSTPSICHAPGGEYFVEGETWNIDSCTQCTCHSGRVLCETEVCPPLLCQNPSRTQDSCCPQCTDQPFRPSLSRNNSVPNYCKNDEGDIFLAAESWKPDVCTSCICIDSVISCFSESCPSVSCERPVLRKGQCCPYCIEDTIPKKVVCHFSGKAYADEERWDLDSCTHCYCLQGQTLCSTVSCPPLPCVEPINVEGSCCPMCPEMYVPEPTNIPIEKTNHRGEVDLEVPLWPTPSENDIVHLPRDMGHLQVDYRDNRLHPSEDSSLDSIASVVVPIIICLSIIIAFLFINQKKQWIPLLCWYRTPTKPSSLNNQLVSVDCKKGTRVQVDSSQRMLRIAEPDARFSGFYSMQKQNHLQADNFYQTV

SEQ ID NO: 81 - protein, 960 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVESLSLLKDFFTSGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 82 - protein, 977 aa, Homo sapiens
MKPSIAEMLHRGRMLWIILLSTIALGWTTPIPLIEDSEEIDEPCFDPCYCEVKESLFHIHCDSKGFTNISQITEFWSRPFKLYLQRNSMRKLYTNSFLHLNNAVSINLGNNALQDIQTGAFNGLKILKRLYLHENKLDVFRNDTFLGLESLEYLQADYNVIKRIESGAFRNLSKLRVLILNDNLIPMLPTNLFKAVSLTHLDLRGNRLKVLFYRGMLDHIGRSLMELQLEENPWNCTCEIVQLKSWLERIPYTALVGDITCETPFHFHGKDLREIRKTELCPLLSDSEVEASLGIPHSSSSKENAWPTKPSSMLSSVHFTASSVEYKSSNKQPKPTKQPRTPRPPSTSQALYPGPNQPPIAPYQTRPPIPIICPTGCTCNLHINDLGLTVNCKERGFNNISELLPRPLNAKKLYLSSNLIQKIYRSDFWNFSSLDLLHLGNNRISYVQDGAFINLPNLKSLFLNGNDIEKLTPGMFRGLQSLHYLYFEFNVIREIQPAAFSLMPNLKLLFLNNNLLRTLPTDAFAGTSLARLNLRKNYFLYLPVAGVLEHLNAIVQIDLNENPWDCTCDLVPFKQWIETISSVSVVGDVLCRSPENLTHRDVRTIELEVLCPEMLHVAPAGESPAQPGDSHLIGAPTSASPYEFSPPGGPVPLSVLILSLLVLFFSAVFVAAGLFAYVLRRRRKKLPFRSKRQEGVDLTGIQMQCHRLFEDGGGGGGGSGGGGRPTLSSPEKAPPVGHVYEYIPHPVTQMCNNPIYKPREEEEVAVSSAQEAGSAERGGPGTQPPGMGEALLGSEQFAETPKENHSNYRTLLEKEKEWALAVSSSQLNTIVTVNHHHPHHPAVGGVSGVVGGTGGDLAGFRHHEKNGGVVLFPPGGGCGSGSMLLDRERPQPAPCTVGFVDCLYGTVPKLKELHVHPPGMQYPDLQQDARLKETLLFSAGKGFTDHQTQKSDYLELRAKLQTKPDYLEVLEKTTYRF

SEQ ID NO: 83 - protein, 313 aa, Homo sapiens
MKSPPLLSPCLSWKRMAGIFFLPFISSGFAPRFKQEENFMLGRAHPSQPRFNWSHLTPLELKNRSVGLGTESTGRGKPHFTLEGHKFLIFGGSIHYFRVPREYWRDRLLKLKACGFNTVTTYVPWNLHEPERGKFDFSGNLDLEAFVLMAAEIGLWVILRPGPYICSEMDLGGLPSWLLQDPRLLLRTTNKSFIEAVEKYFDHLIPRVIPLQYRQAGPVIAVQVENEYGSFNKDKTYMPYLHKALLRRGIVELLLTSDGEKHVLSGHTKGVLAAINLQKLHQDTFNQLHKVQGASIDAGLLSDASQAATSSSL

SEQ ID NO: 84 - protein, 468 aa, Homo sapiens
MAKRNAEKELTDRNWDQEDEAEEVGTFSMASEEVLKNRAIKKAKRRNVGFESDTGGAFKGFKGLVVPSGGGRFSGFGSGAGGKPLEGLSNGNNITSAPPFASAKAAADPKVAFGSLAANGPTTLVDKVSNPKTNGDSQQPSSSGLASSKACVGNAYHKQLAALNCSVRDWIVKHVNTNPLCDLTPIFKDYEKYLANIEQQHGNSGRNSESESNKVAAETQSPSLFGSTKLQQESTFLFHGNKTEDTPDKKMEVASEKKTDPSSLGATSASFNFGKKVDSSVLGSLSSVPLTGFSFSPGNSSLFGKDTTQSKPVSSPFPTKPLEGQAEGDSGECKGGDEEENDEPPKVVVTEVKEEDAFYSKKCKLFYKKDNEFKEKGIGTLHLKPTANQKTQLLVRADTNLGNILLNVLIPPNMPCTRTGKNNVLIVCVPNPPIDEKNATMPVTMLIRVKTSEDADELHKILLEKKDA

SEQ ID NO: 85 - protein, 967 aa, Homo sapiens
MAARGRGLLLLTLSVLLAAGPSAAAAKLNIPKVLLPFTRATRVNFTLEASEGCYRWLSTRPEVASIEPLGLDEQQCSQKAVVQARLTQPARLTSIIFAEDITTGQVLRCDAIVDLIHDIQIVSTTRELYLEDSPLELKIQALDSEGNTFSTLAGLVFEWTIVKDSEADRFSDSHNALRILTFLESTYIPPSYISEMEKAAKQGDTILVSGMKTGSSKLKARIQEAVYKNVRPAEVRLLILENILLNPAYDVYLMVGTSIHYKVQKIRQGKITELSMPSDQYELQLQNSIPGPEGDPARPVAVLAQDTSMVTALQLGQSSLVLGHRSIRMQGASRLPNSTIYVVEPGYLGFTVHPGDRWVLETGRLYEITIEVFDKFSNKVYVSDNIRIETVLPAEFFEVLSSSQNGSYHRIRALKRGQTAIDAALTSVVDQDGGVHILQVPVWNQQEVEIHIPITLYPSILTFPWQPKTGAYQYTIRAHGGSGNFSWSSSSHLVATVTVKGVMTTGSDIGFSVIQAHDVQNPLHFGEMKVYVIEPHSMEFAPCQVEARVGQALELPLRISGLMPGGASEVVTLSDCSHFDLAVEVENQGVFQPLPGRLPPGSEHCSGIRVKAEAQGSTTLLVSYRHGHVHLSAKITIAAYLPLKAVDPSSVALVTLGSSKEMLFEGGPRPWILEPSKFFQNVTAEDTDSIGLALFAPHSSRNYQQHWILVTCQALGEQVIALSVGNKPSLTNPFPAVEPAVVKFVCAPPSRLTLAPVYTSPQLDMSCPLLQQNKQVVPVSSHRNPRLDLAAYDQEGRRFDNFSSLSIQWESTRPVLASIEPELPMQLVSQDDESGQKKLHGLQAILVHEASGTTAITATATGYQESHLSSARTKQPHDPLVPLSASIELILVEDVRVSPEEVTIYNHPGIQAELRIREGSGYFFLNTSTADVVKVAYQEARGVAMVSLGHRSPLLVFIPYLGCCVVN

SEQ ID NO: 86 - protein, 990 aa, Homo sapiens
MSEETRQSKLAAAKKKLREYQQRNSPGVPTGAKKKKKIKNGSNPETTTSGGCHSPEDTPKDNAATLQPSDDTVLPGGVPSPGASLTSMAASQNHDADNVPNLMDETKTFSSTESLRQLSQQLNGLVCESATCVNGEGPASSANLKDLESRYQQLAVALDSSYVTNKQLNITIEKLKQQNQEITDQLEEEKKECHQKQGALREQLQVHIQTIGILVSEKAELQTALAHTQHAARQKEGESEDLASRLQYSRRRVGELERALSAVSTQQKKADRYNKELTKERDALRLELYKNTQSNEDLKQEKSELEEKLRVLVTEKAGMQLNLEELQKKLEMTELLLQQFSSRCEAPDANQQLQQAMEERAQLEAHLGQVMESVRQLQMERDKYAENLKGESAMWRQRMQQMSEQVHTLREEKECSMSRVQELETSLAELRNQMAEPPPPEPPAGPSEVEQQLQAEAEHLRKELEGLAGQLQAQVQDNEGLSRLNREQEERLLELERAAELWGEQAEARRQILETMQNDRTTISRALSQNRELKEQLAELQSGFVKLTNENMEITSALQSEQHVKRELGKKLGELQEKLSELKETVELKSQEAQSLQQQRDQYLGHLQQYVAAYQQLTSEKEVLHNQLLLQTQLVDQLQQQEAQGKAVAEMARQELQETQERLEAATQQNQQLRAQLSLMAHPGEGDGLDREEEEDEEEEEEEAVAVPQPMPSIPEDLESREAMVAFFNSAVASAEEEQARLRGQLKEQRVRCRRLAHLLASAQKEPEAAAPAPGTGGDSVCGETHRALQGAMEKLQSRFMELMQEKADLKERVEELEHRCIQLSGETDTIGEYIALYQSQRAVLKERHREKEEYISRLAQDKEEMKVKLLELQELVLRLVGDRNEWHGRFLAAAQNPADEPTSGAPAPQELGAANQQGDLCEVSLAGSVEPAQGEAREGSPRDNPTAQQIMQLLREMQNPRERPGLGSNPCIPFFYRADENDEVKITVI

SEQ ID NO: 87 - protein, 655 aa, Homo sapiens
MPLKWKTSSPAIWKFPVPVLKTSRSTPLSPAYISLVEEEDQHMKLSLGGSEMGLSSHLQSSKAGPTRIFTSNTHSSVVLQGFDQLRLEGLLCDVTLMPGDTDDAFPVHRVMMASASDYFKAMFTGGMKEQDLMCIKLHGVSKVGLRKIIDFIYTAKLSLNMDNLQDTLEAASFLQILPVLDFCKVFLISGVTLDNCVEVGRIANTYNLTEVDKYVNSFVLKNFPALLSTGEFLKLPFERLAFVLSSNSLKHCTELELFKATCRWLRLEEPRMDFAAKLMKNIRFPLMTPQELINYVQTVDFMRTDNTCVNLLLEASNYQMMPYMQPVMQSDRTAIRSDTTHLVTLGGVLRQQLVVSKELRMYDEKAHEWKSLAPMDAPRYQHGIAVIGNFLYVVGGQSNYDTKGKTAVDTVFRFDPRYNKWMQVASLNEKRTFFHLSALKGYLYAVGGRNAAGELPTVECYNPRTNEWTYVAKMSEPHYGHAGTVYGGVMYISGGITHDTFQKELMCFDPDTDKWIQKAPMTTVRGLHCMCTVGERLYVIGGNHFRGTSDYDDVLSCEYYSPILDQWTPIAAMLRGQSDVGVAVFENKIYVVGGYSWNNRCMVEIVQKYDPDKDEWHKVFDLPESLGGIRACTLTVFPPEETTPSPSRESPLSAP

SEQ ID NO: 88 - protein, 897 aa, Homo sapiens
MERAGATSRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 89 - protein, 919 aa, Homo sapiens
MQRSIMSFFHPKKEGKAKKPEKEASNSSRETEPPPKAALKEWNGVVSESDSPVKRPGRKAARVLGSEGEEEDEALSPAKGQKPALDCSQVSPPRPATSPENNASLSDTSPMDSSPSGIPKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTY

SEQ ID NO: 90 - protein, 296 aa, Homo sapiens
MGKKSRVKTQKSGTGATATVSPKEILNLTSELLQKCSSPAPGPGKEWEEYVQIRTLVEKIRKKQKGLSVTFDGKREDYFPDLMKWASENGASVEGFEMVNFKEEGFGLRATRDIKAEELFLWVPRKLLMTVESAKNSVLGPLYSQDRILQAMGNIALAFHLLCERASPNSFWQPYIQTLPSEYDTPLYFEEDEVRYLQSTQAIHDVFSQYKNTARQYAYFYKVIQTHPHANKLPLKDSFTYEDYRWAVSSVMTRQNQIPTEDGSRVTLALIPLWDMCNHTNGLTPEDSFALAVASA

SEQ ID NO: 91 - protein, 463 aa, Homo sapiens
MATFPCQLCGKTFLTLEKFTIHNYSHSRERPYKCVQPDCGKAFVSRYKLMRHMATHSPQKSHQCAHCEKTFNRKDHLKNHLQTHDPNKMAFGCEECGKKYNTMLGYKRHLALHAASSGDLTCGVCALELGSTEVLLDHLKAHAEEKPPSGTKEKKHQCDHCERCFYTRKDVRRHLVVHTGCKDFLCQFCAQRFGRKDHLTRHTKKTHSQELMKESLQTGDLLSTFHTISPSFQLKAAALPPFPLGASAQNGLASSLPAEVHSLTLSPPEQAAQPMQPLPESLASLHPSVSPGSPPPPLPNHKYNTTSTSYSPLASLPLKADTKGFCNISLFEDLPLQEPQSPQKLNPGFDLAKGNAGKVNLPKELPADAVNLTIPASLDLSPLLGFWQLPPPATQNTFGNSTLALGPGESLPHRLSCLGQQQQEPPLAMGTVSLGQLPLPPIPHVFSAGTGSAILPHFHHAFR

SEQ ID NO: 92 - protein, 297 aa, Homo sapiens
MKTLSPDRIQPHIMTDVPATFTQAECNGDKPPENGQQTITKISEELTDVDSPLPHYRVEPSLEGALTKGSQEERRKLQGNMLLNSSMEDKMLKENPEEKLFIVHKAITDLSLQETSADEMTFREGHQWEKIPLSGSNQEIRRQKERITEQPLKEEEDEDRKNKGHQAAEIEWLGFRKPSQADMLHSKHDEEQKVWDEEIDDDDDDNCNNDEDEVRVIEFKKKHEEVSQFKEEGDASEDSPLSSASSQAVTPDEQPTLGKKSDISRNAYSRYNTISYRKIRKGNTKQRIDEFESMMHL

SEQ ID NO: 93 - protein, 1012 aa, Homo sapiens
MRWGLRPRGPGAAALATARSLWGTPRLPCSPGWQGATKRLLVRSVSGASNHQPNSNSGRYRDTVLLPQTSFPMKLLGRQQPDTELEIQQKCGFSELYSWQRERKVKTEFCLHDGPPYANGDPHVGHALNKILKDIANRFHMMNGSKIHFVPGWDCHGLPIEIKVLSELGREAQNLSAMEIRKKARSFAKAAIEKQKSAFIRWGIMADWNNCYYTFDGKYEAKQLRTFYQMYDKGLVYRSYKPVFWSPSSRTALAEAELEYNPEHVSRSIYVKFPLLKPSPKLASLIDGSSPVSILVWTTQPWTIPANEAVCYMPESKYAVVKCSKSGDLYVLAADKVASVASTLETTFETISTLSGVDLENGTCSHPLIPDKASPLLPANHVTMAKGTGLVHTAPAHGMEDYGVASQHNLPMDCLVDEDGVFTDVAGPELQNKAVLEEGTDVVIKMLQTAKNLLKEEKLVHSYPYDWRTKKPVVIRASKQWFINITDIKTAAKELLKKVKFIPGSALNGMVEMMDRRPYWCISRQRVWGVPIPVFHHKTKDEYLINSQTTEHIVKLVEQHGSDIWWTLPPEQLLPKEVLSEVGGPDALEYVPGQDILDIWFDSGTSWSYVLPGPDQRADLYLEGKDQLGGWFQSSLLTSVAARKRAPYKTVIVHGFTLGEKGEKMSKSLGNVIHPDVVVNGGQDQSKEPPYGADVLRWWVADSNVFTEVAIGPSVLNAARDDISKLRNTLRFLLGNVADFNPETDSIPVNDMYVIDQYMLHLLQDLANKITELYKQYDFGKVVRLLRTFYTRELSNFYFSIIKDRLYCEKENDPKRRSCQTALVEILDVIVRSFAPILPHLAEEVFQHIPYIKEPKSVFRTGWISTSSIWKKPGLEEAVESACAMRDSFLGSIPGKNAAEYKVITVIEPGLLFEIIEMLQSEETSSTSQLNELMMASESTLLAQEPREMTADVIELKGKFLINLEGGDIREESSYKVIVMPTTKEKCPRCWKYTAESSDTLCPRCAEVVSGK

SEQ ID NO: 94 - protein, 296 aa, Homo sapiens
MMLPLQGAQMLQMLEKSLRKSLPASLKVYGTVFHINHGNPFNLKAVVDKWPDFNTVVVCPQEQDMTDDLDHYTNTYQIYSKDPQNCQEFLGSPELINWKQHLQIQSSQPSLNEAIQNLAAIKSFKVKQTQRILYMAAETAKELTPFLLKSKILSPSGGKPKAINQEMFKLSSMDVTHAHLVNKFWHFGGNERSQRFIERCIQTFPTCCLLGPEGTPVCWDLMDQTGEMRMAGTLPEYRLHGLVTYVIYSHAQKLGKLGFPVYSHVDYSNEAMQKMSYTLQHVPIPRSWNQWNCVPL

SEQ ID NO: 95 - protein, 305 aa, Homo sapiens
MLTLTFLLVGLLNHQWLKETDVPQKSRQLYAIIAEYGSRLYKYQARLRMPKEQLELLKKESQNLENNFRQILFLVEQIDVLKALLRDMKDGMDNNHNWNTHGDPVEDPDHTEEVSNLVNYVLKKLREDQVEMADYALKSAGASIIEAGTSESYKNNKAKLYWHGIGFLNHEMPPDIILQPDVYPGKCWAFPGSQGHTLIKLATKIIPTAVTMEHISEKVSPSGNISSAPKEFSVYGITKKCEGEEIFLGQFIYNKTGTTVQTFELQHAVSEYLLCVKLNIFSNWGHPKYTCLYRFRVHGTPGKHI

SEQ ID NO: 96 - protein, 912 aa, Homo sapiens
MSVELLRSVNDFLWLGAALEGLCSASVIYHYPGGTGGKSGARRFQGSTLPAEAANRHRPGALTTNGINPDTSTEIGRAKNCLSPEDIIDKYKEAISYYSKYKNAGVIELEACIKAVRVLAIQKRSMEASEFLQNAVYINLRQLSEEEKIQRYSILSELYELIGFHRKSAFFKRVAAMQCVAPSIAEPGWRACYKLLLETLPGYSLSLDPKDFSRGTHRGWAAVQMRLLHELVYASRRMGNPALSVRHLSFLLQTMLDFLSDQEKKDVAQSLENYTSKCPGTMEPIALPGGLTLPPVPFTKLPIVRHVKLLNLPASLRPHKMKSLLGQNVSTKSPFIYSPIIAHNRGEERNKKIDFQWVQGDVCEVQLMVYNPMPFELRVENMGLLTSGVEFESLPAALSLPAESGLYPVTLVGVPQTTGTITVNGYHTTVFGVFSDCLLDNLPGIKTSGSTVEVIPALPRLQISTSLPRSAHSLQPSSGDEISTNVSVQLYNGESQQLIIKLENIGMEPLEKLEVTSKVLTTKEKLYGDFLSWKLEETLAQFPLQPGKVATFTINIKVKLDFSCQENLLQDLSDDGISVSGFPLSSPFRQVVRPRVEGKPVNPPESNKAGDYSHVKTLEAVLNFKYSGGPGHTEGYYRNLSLGLHVEVEPSVFFTRVSTLPATSTRQCHLLLDVFNSTEHELTVSTRSSEALILHAGECQRMAIQVDKFNFESFPESPGEKGQFANPKQLEEERREARGLEIHSKLGICWRIPSLKRSGEASVEGLLNQLVLEHLQLAPLQWDVLVDGQPCDREAVAACQVGDPVRLEVRLTNRSPRSVGPFALTVVPFQDHQNGVHNYDLHDTVSFVGSSTFYLDAVQPSGQSACLGALLFLYTGDFFLHIRFHEDSTSKELPPSWFCLPSVHVCALEAQA

SEQ ID NO: 97 - protein, 14 aa, Homo sapiens
TLQQFPGGSIDLQK

SEQ ID NO: 98 - protein, 13 aa, Homo sapiens
ENSDIPILFSLQR

SEQ ID NO: 99 - protein, 14 aa, Homo sapiens
VYALPEDLVEVKPK

SEQ ID NO: 100 - protein, 14 aa, Homo sapiens
IYALPDDLVEVKPK

SEQ ID NO: 101 - protein, 11 aa, Homo sapiens
DIMLANQPSPR

SEQ ID NO: 102 - protein, 12 aa, Homo sapiens
LVCAVDSNPPAR

SEQ ID NO: 103 - protein, 11 aa, Homo sapiens
IHTGEKPYDCK

SEQ ID NO: 104 - protein, 11 aa, Homo sapiens
VHTGEKPYECK

SEQ ID NO: 105 - protein, 14 aa, Homo sapiens
DGHCNVQFINVGEK

SEQ ID NO: 106 - protein, 13 aa, Homo sapiens
TNHSNIMDDPFIR

SEQ ID NO: 107 - protein, 11 aa, Homo sapiens
EAGEQGDIEPR

SEQ ID NO: 108 - protein, 14 aa, Homo sapiens
GPGAGEGPGGAFAR

SEQ ID NO: 109 - protein, 11 aa, Homo sapiens
ASIHEAWTDGK

SEQ ID NO: 110 - protein, 10 aa, Homo sapiens
FSIEYLSEGR

SEQ ID NO: 111 - protein, 10 aa, Homo sapiens
HFLEAIENNK

SEQ ID NO: 112 - protein, 10 aa, Homo sapiens
INHEPTTFQK

SEQ ID NO: 113 - protein, 11 aa, Homo sapiens
DELGEQVLGLK

SEQ ID NO: 114 - protein, 11 aa, Homo sapiens
TILESQDPLGK

SEQ ID NO: 115 - protein, 10 aa, Homo sapiens
DLFSTFTITR

SEQ ID NO: 116 - protein, 10 aa, Homo sapiens
LNSELQQQLK

SEQ ID NO: 117 - protein, 11 aa, Homo sapiens
ILDLLNEGSAR

SEQ ID NO: 118 - protein, 12 aa, Homo sapiens
AQEAAATQLGLK

SEQ ID NO: 119 - protein, 10 aa, Homo sapiens
NHLTNPCYPR

SEQ ID NO: 120 - protein, 12 aa, Homo sapiens
ASAYVFASPGCK

SEQ ID NO: 121 - protein, 11 aa, Homo sapiens
HFGSVVAQTIR

SEQ ID NO: 122 - protein, 10 aa, Homo sapiens
IEDILEVIEK

SEQ ID NO: 123 - protein, 11 aa, Homo sapiens
EVNTVLADVIK

SEQ ID NO: 124 - protein, 10 aa, Homo sapiens
SVYLYLSSLR

SEQ ID NO: 125 - protein, 10 aa, Homo sapiens
IHNHLPEIQK

SEQ ID NO: 126 - protein, 10 aa, Homo sapiens
FEGYCIDLLK

SEQ ID NO: 127 - protein, 12 aa, Homo sapiens
APGIPSGMETLK

SEQ ID NO: 128 - protein, 10 aa, Homo sapiens
MPIDLVIDER

SEQ ID NO: 129 - protein, 9 aa, Homo sapiens
YMAVIQYGK

SEQ ID NO: 130 - protein, 11 aa, Homo sapiens
AVLITDQSILK

SEQ ID NO: 131 - protein, 10 aa, Homo sapiens
YPLHAIMEIK

SEQ ID NO: 132 - protein, 11 aa, Homo sapiens
MTISVALPQLK

SEQ ID NO: 133 - protein, 7 aa, Artificial
  N-terminal pET-DEST42 vector encoded sequences
MYKKAGT

SEQ ID NO: 134 - protein, 4 aa, Artificial
  N-terminal pET-DEST42 vector encoded sequences
MYKK

SEQ ID NO: 135 - protein, 4 aa, Artificial
  N-terminal pET-DEST42 vector encoded sequences
KAGT

(19) United States
(12) Patent Application Publication
(10) Pub. No.: US 2008/0145885 A1
(43) Pub. Date: Jun. 19, 2008

(54) Proteome Standards for Mass Spectrometry

(75) Inventors: Alexander William Bell, Montreal (CA); John J.M. Bergeron, Pointe Clarie (CA); Thomas Chappell, San Marcos, CA (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/776,537

(22) Filed: Jul. 11, 2007

Related U.S. Application Data
(60) Provisional application No. 60/830,202, filed on Jul. 11, 2006.
(60) Provisional application No. 60/868,309, filed on Dec. 1, 2006.

(51) Int. Cl. C12Q 1/37; G01N 31/00; G06Q 30/00

(52) U.S. Cl. 435/23; 436/15; 705/1

(57) ABSTRACT

The invention relates generally to standard sets of proteins, polypeptides, or peptides, and methods of using the standard sets to standardize laboratories, laboratory procedures, or laboratory equipment, and to certify laboratories and laboratory technicians. In certain aspects, the invention relates to standard sets of proteins, polypeptides, or peptides, that may be used in mass spectrometry.

### CROSS REFERENCE TO RELATED APPLICATIONS

The present application claims benefit of priority to U.S. provisional application 60/830,202 filed Jul. 11, 2006, and to U.S. provisional application 60/868,309 filed Dec. 1, 2006, both entitled “Proteome Standards for Mass Spectrometry”, and both of which are incorporated by reference herein in their entireties.

### REFERENCE TO A SEQUENCE LISTING

This application incorporates by reference a Sequence Listing submitted with this application as text file IVGN629ST25.txt created on Jul. 11, 2007 and having a size of 503,808 bytes.

### FIELD OF THE INVENTION

The invention relates generally to standard sets of proteins, polypeptides, or peptides, and methods of using the standard sets to standardize laboratories, laboratory procedures, or laboratory equipment for protein analysis and identification, and to certify laboratories and laboratory technicians in protein analysis and identification. In certain aspects, the invention relates to standard sets of proteins, polypeptides, or peptides, that may be used in mass spectrometry.

### BACKGROUND OF THE INVENTION

Many diseases caused by genetic mutations or environmental factors may be associated with changes in the amino acid sequence or relative abundance of a protein. Proteins encoded by the human genome (collectively referred to as the human proteome) may be identified by determining the sequences of the open reading frames (ORFs) of the genome. Characterization of the human proteome permits the use of analytical techniques such as mass spectrometry to determine changes in the sequence or relative abundance of a protein in an individual, associate the changes with particular diseases and conditions, and ultimately, to diagnose diseases or medical conditions.

Identifying differences in the sequence or relative abundances of proteins may also lead to a better understanding of particular diseases and to potentially more effective therapies for such diseases. Indeed, personalized therapy regimens based on a patient's particular protein make-up can result in life saving medical interventions. Novel drugs or compounds can be discovered that interact with protein variants, or to modify the amount of certain proteins.

Various methods are used for analyzing the sequence, size, and relative abundance of proteins. In one example, gel electrophoresis or two-dimensional gel electrophoresis may be used. In the human genome, because so many of the ORFs result in a proteins of similar size, there is much molecular weight overlap, and gel electrophoresis is not as useful for distinguishing different proteins having the same molecular weight.

Mass spectrometry has been used in the biosciences to analyze protein and nucleic acid samples. MALDI-MS requires incorporation of the macromolecule to be analyzed in a matrix, and has been performed on polypeptides and on nucleic acids mixed in a solid (i.e., crystalline) matrix. In these methods, a laser is used to strike the biopolymer/matrix mixture, which is crystallized on a probe tip, thereby effecting desorption and ionization of the biopolymer.

Proteins of the human proteome, when analyzed using mass spectrometry, have been found to cluster at certain molecular weights, making it more difficult to identify the individual proteins. The instruments used in the analysis must be sensitive enough to distinguish between proteins in a cluster, and to determine their relative abundance. Also, the laboratory technician must be skilled enough to run the analysis of the proteins or protein fragments to determine their identities, and the analysis methods, such, as, for example, computer analysis methods, must be robust enough to distinguish between closely associated peaks.

Where more than one laboratory is involved in analyzing the human proteome, it is important that the various laboratory instruments, the methods used to analyze the results, the laboratory protocols, and, potentially, the laboratory technicians, are standardized so that results in one laboratory, or even in the same laboratory using different machine or technicians, may be compared. This is important also where a single laboratory wants to assess the quality and sensitivity of its own instruments.

### SUMMARY OF THE INVENTION

The present invention provides standard sets of proteins, polypeptides, or peptides, and methods of using the standard sets to standardize laboratories, laboratory procedures, or laboratory equipment, and to certify laboratory technicians and laboratories. The present invention provides a set of proteins of known quantity and amino acid sequence for calibrating the sensitivity and accuracy of laboratory equipment such as, for example, mass spectrometers and associated sequence analysis programs.

In one embodiment of the invention, multiple proteins of known quantity are provided as a standard set, in which at least 2, 3, 4, 5, 6, 7, 8, 9, 10, 15, 20, 25, 30, 35, 40, 45, 50, 55, 60, 65, 70, 75, 80, 85, 90, 95, or 100 proteins have different molecular weights. For example, the set can have 2, 3, 4, 5, 6, 7, 8, 9, 10, between 10 and 15, between 15 and 20, between 20 and 25, between 25 and 30, between 30 and 35, between 35 and 40, between 40 and 45, between 45 and 50, between 50 and 55, between 55 and 60, between 60 and 65, between 65 and 70, between 70 and 75, between 75 and 80, between 80 and 85, between 85 and 90, between 90 and 95, or between 95 and 100, or more than 100 proteins that have different molecular weights.

In another embodiment, multiple proteins of known quantity are provided as a standard set, in which at least two proteins are present in quantities that differ by at least 10%, 20% 30%, 40%, 50%, 60%, 70%, 80%, or 90%, or by at least 2, 3, 4, 5, 6, 7, 8, 9, 10, 15, 20, 25, 30, 35, 40, 45, 50, 55, 60, 65, 70, 75, 80, 85, 90, 95, or 100 fold.

In exemplary embodiments of the invention, the standard sets may be used for whole protein mass spectrometry analysis, for example, using MALDI MS, or can be used for analysis of peptides generated by digestion or hydrolysis of the proteins of the proteome set. Digestion may be performed, for example, by proteases, such as, for example, trypsin.

In another embodiment of the invention, methods are provided of standardizing a mass spectrometer and mass spectrometry analysis methods using proteome standards of the invention. Also provided are methods of standardizing multiple mass spectrometers and mass spectrometry analysis methods using proteome standards to enable collaborative analysis of proteomes of organisms, cells, and tissues.

Provided in the present invention is a standard set for mass spectrometry, comprising a plurality of proteins, in which when the plurality of proteins are digested with one or more proteases or proteolytic agents, no two proteolytic fragments having molecular weights between 700 and 4800 Da that are generated by digestion of different proteins are identical in amino acid sequence, and in which when the plurality of proteins are digested with the one or more proteases and analyzed by mass spectrometry, at least five proteolytic fragments derived from the plurality of proteins form a subset of proteolytic fragments in which the mass peak produced by each proteolytic fragment of the subset differs from every other mass peak produced by a member of the subset by no more than 10 Da.

Also provided in the present invention is a polypeptide standard set for mass spectrometry comprising a plurality of polypeptides, in which each polypeptide of the plurality of polypeptides comprises one or more unique peptide segments ranging in size from between about 700 to about 4800 Da bordered by protease cleavage sites or bordered by a protease cleavage site and the N- or C-terminus of the polypeptide that comprises the segment, in which the plurality of polypeptides in aggregate comprise a subset of at least five unique fragments bordered by cleavage sites of the protease, or bordered by a cleavage site of the protease and a terminus of the polypeptide that comprises the fragment, in which each peptides of the subset differs from each of the other peptides of the subset by no more than 10 Da. In some exemplary embodiments, the polypeptide standard set includes a plurality of polypeptides that in aggregate have at least 3, 4, 5, 6, 7, 8, 9, or 10 peptide segments bordered by protease cleavage sites (or a cleavage site and either an N-terminus or C-terminus of the polypeptide), of each of at least two molecular weight range subsets, wherein each of the peptide segments of a molecular weight range subset differs from another peptide segment of the same molecular weight range subset by no more than 10 Da.

In exemplary embodiments of the present invention, the plurality of proteins or polypeptides are from a single species of organism, for example, from a plant species, animal species, fungal species, or bacteria species, for example a mammalian species, for example, a human. In exemplary embodiments of the present invention, the species is human, mouse, rat, dog, chimpanzee, gorilla, rhesus monkey, macaque, cow, horse, chicken, zebrafish, pufferfish, a Drosphila species, or a yeast species.

The standard set may comprise, for example, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more proteins of a single species of organism. The standard set may, for example, comprise 3, 4, 5, 6, 7, 8, 9, 10, 11, 12, 13, 14, 15, 16, 17, 18, 19, 20, 21, 22, 23, 24, 25, 26, 27, 28, 29, 30, or more proteins of a single species of organism, in which when the plurality of proteins are digested with the protease and analyzed by mass spectrometry, at least 3, 4, 5, 6, 7, 8, 9, 10, 11, 12, 13, 14, 15, 16, 17, 18, 19, or 20 proteolytic fragments derived from the plurality of proteins form a molecular weight peptide subset in which the generated peptides of the subset produce mass peaks that each differ from one another by lno more than 10 Da. In certain embodiments of the invention, the standard set comprises at least twenty proteins of a single species, in which when the plurality of proteins are digested with a protease and analyzed by mass spectrometry, the mass spectrum produced has at least one region which has at least five proteolytic fragment peaks that each differ from one another by no more than 10 Da. In some preferred embodiments, a proteome standard set has 3, 4, 5, 6, 7, 8, 9, 10, 11, 12, 13, 14, 15, 16, 17, 18, 19, 20, or more proteins of a single species of organism, in which each of the proteins of the standard set when digested with a protease, produces a fragment that is within 10 Da of a proteolytic fragment of each of the other proteins of the standard set. In these embodiments, mass spectrometry of a proteolytic digest of the proteome standard set produces at least one region of peaks (or peak cluster) in which a proteolytic fragment of each of the proteins of the set is present, in which the proteolytic fragments of the peak or cluster in the mass spectrum do not differ in molecular weight from one another by more than 10 Da. In exemplary embodiments, at least one of the regions of the mass spectrum spans a molecular weight range of between about 1200 and about 1210 Daltons. In certain embodiments of the invention, when the plurality of proteins are digested with a protease at least twenty proteolytic fragments are produced that have a molecular weight from about 1200 to about 1210 Daltons. Also provided in the present invention is a standard set comprising proteins or polypeptides, which, when digested with trypsin, comprise a set of at least 3, 4, 5, 6, 7, 8, 9, 10, 11, 12, 13, 14, 15, 16, 17, 18, 19, 20, 21, 22, 23, 24, 25, or 26 of the peptide fragments of FIG. 3.

In further embodiments of the present invention is a standard set comprising between twenty and thirty proteins, in which when the plurality of proteins are digested with a protease and analyzed by mass spectrometry, the mass spectrum produced has at least one region that has at least twenty proteolytic fragments derived from the plurality of proteins produce mass peaks that each differ from one another by less than 10 Da. In other embodiments of the present invention are provided a standard set comprising between 30 and 50, or at least 50, 55, 60, 75, 70, 75, 80, 85, 90, 95, 100, 110, 120, 130, or 140 proteins, in which when the plurality of proteins are digested with a protease and analyzed by mass spectrometry, the mass spectrum produced has at least two regions that each have at least twenty proteolytic fragments derived from the plurality of proteins produce mass peaks in which each peak differs from each of the others by no more than 10 Da.

In other exemplary embodiments of the present invention are provided standard sets where at least two of the proteins have a molecular weight of between about 10 kDa and about 200 kDa. Also provided are standard sets in which at least two of the proteins have molecular weights that are within 2, 3, 4, 5, 6, 7, 8, 9, or 10 kDa of one another. For example, standard sets can comprise a plurality of proteins in which at least four of the proteins may have molecular weights of between 30 and 40 kDa and differ by 5 kDa or less, or can comprise a plurality of proteins in which at least four of the proteins have molecular weights of between 40 and 60 kDa and differ by 5 kDa or less, or can comprise a plurality of proteins in which at least four of the proteins have molecular weights of between 60 and 80 kDa and differ by 7 kDa or less, or can comprise a plurality of proteins in which at least four of the proteins have molecular weights of between 80 and 150 kDa and differ by 15 kDa or less.

In yet other embodiments of the present invention are standard sets in which at least four of the proteins have molecular weights of less than 100 kDa, in which at least two, at least three, or at least four of the proteins having a molecular weight of less than 100 kDa differs in molecular weight from at least two other proteins of the set by 4 kDa or less. Also provided in the present invention are standard sets in which at least four of the proteins have molecular weights of 100 kDa or greater, in which at least two, at least three, or at least four of the proteins having a molecular weight of 100 kDa or greater differs in molecular weight from at least two other proteins of the set by 15 kDa or less.

In some exemplary embodiments of the present invention, the plurality of proteins or polypeptides of the standard set are present at concentrations that are within 1, 2, 3, 4, 5, 6, 7, 8, 9, or 10% of one another. In some examples, at least one protein of the plurality of proteins is present at a concentrations that is 10, 9, 8, 7, 6, 5, 4, 3, 2, 1% or less than the concentration of at least one other protein of the plurality of proteins.

The standard sets as described herein can be provided in kits, in which the proteins of the set are provided as one or more lyophilates, or in liquid form. When provided in liquid form, the protein standards can be provided in frozen form. The kits can provide all of the standards of the set in a single tube, vial, or other container, or can provide different proteins of the set in two or more different containers, such as tubes or vials. The kit can further include additional reagents, such as but not limited to, one or more proteases or protein cleavage reagents, a gel loading buffer, a solvent or buffer compatible with mass spectrometry, or a mass spectrometry matrix., such as for example, sinapinic acid (SA) or alpha-cyano-4-hydroxycinnamic acid (CHCA), or a matrix additive such as a mass spectrometry-compatible solubilizer, mass spectrometry-compatible sorbent, or a mass spectrometry-compatible buffer. MS-compatible solubilizers, additives, buffers, and sorbents, as well as matrix materials for MALDI MS are known in the art and disclosed in co-pending U.S. patent application Ser. No. 11/258,363 (U.S. Patent application publication US-2006-0214104-A1) and co-pending U.S. patent application Ser. No. 11/131,744 U.S. Patent application publication US-2006-0238808-A 1) both of which are herein incorporated by reference in their entireties.

Also provided in the present invention are kits comprising two polypeptide standard sets for mass spectrometry, a first standard set comprising a plurality of polypeptides, in which each polypeptide of the plurality of polypeptides comprises unique 700 to 4800 Da peptide segments bordered by protease cleavage sites, or bordered by a protease cleavage site and either the N-terminus or C-terminus of a polypeptide of the set, and further in which the plurality of polypeptides comprise at least five unique fragments bordered by cleavage sites of the protease, or bordered by a protease cleavage site and either the N-terminus or C-terminus of a polypeptide of the set, that differ from one another by 10 Da or less; and a second standard set comprising the plurality of proteins, in which at least one of the plurality of proteins is present at a different concentration in the second standard set than the first standard set. The first set can optionally have all proteins of the set present at the same concentration. The second set can optionally have all proteins of the set present at the same concentration. The kit may further include instructions for use.

In other exemplary embodiments of the present invention are provided methods for standardizing laboratories and/or laboratory procedures, comprising separating the polypeptides or proteins of the standard sets using electrophoresis or chromatography, isolating a plurality or all of said separated polypeptides or proteins, proteolytically cleaving the isolated separated polypeptides or proteins to generate protease fragments, and analyzing the protease fragments. In exemplary embodiments, the analysis is performed using mass spectrometry. In further embodiments, the results of the analysis are compared to a reference set of results, to determine whether said laboratory and/or laboratory procedure meets an objective standard of quality. The results may include identification of the proteins of the proteome standard set. In certain embodiments, the laboratory and/or laboratory procedure meets the standard of quality where the results of the protease fragment analysis differs from the reference set of results by not more than 1, 2, 3, 4, 5, 6, 7, 8, 9, or 10%.

Also provided in the present invention is a method for certifying a laboratory technician, laboratory, or core facility, comprising providing said technician, laboratory, or core facility with the polypeptides or proteins of the standard sets of the present invention and in which said laboratory technician, laboratory, or core facility obtains proteolytically cleaved polypeptides and analyzes said proteolytically cleaved polypeptides or proteolytically cleaved proteins. In some aspects, the technician, laboratory, or core facility, separates the polypeptides or proteins using electrophoresis or chromatography, isolates a plurality or all of said separated polypeptides or proteins, proteolytically cleaves the isolated separated polypeptides or proteins to generate protease fragments, and analyzes the protease fragments. In exemplary aspects, the analysis is performed using mass spectrometry. The results of the analysis may, for example, be compared to a reference set of results, to determine whether said technician, laboratory, or core facility, is certified. In such aspects, for example, the laboratory technician, laboratory, or core facility is certified where the results of the protease fragment analysis differs from the reference set of results by not more than 1, 2, 3, 4, 5, 6, 7, 8, 9, or 10%.

Also provided in the present invention are kits that include proteome standard sets.

In another aspect the invention provides methods of generating revenue by providing a customer with a proteome standard set in exchange for consideration, such as, for example, money.

### BRIEF DESCRIPTION OF THE DRAWINGS

FIG. 1 provides a schematic diagram of the principle of selecting a standard set that is representative of the proteins in a biological sample in one, two, or more properties.

FIG. 2 presents an example of filtering of tryptic peptides obtained from human proteins, in which tryptic peptides of the same molecular weight but different sequences are accepted.

FIG. 3 presents an example of tryptic peptides closely overlapping in molecular weight.

FIG. 4 is an SDS PAGE gel of 20 individual proteins used in a Proteome Standard Set of the invention.

FIG. 5 is a schematic diagram showing preparation of proteins of a Proteome Standard Set of the invention for mass spectrometry.

FIG. 6 shows results of MS analysis of proteins of a 20 protein Proteome Standard set of the invention.

FIG. 7 a) shows mass spectra of different concentrations of a digested Proteome Standard Set of the invention. b) shows mass spec analysis of different concentrations of a digested Proteome Standard Set of the invention.

FIG. 8 a) is an SDS PAGE gel of proteins of a Proteome Standard set of the invention after incubation at various temperatures. b) shows mass spectra of different concentrations of a digested Proteome Standard Set of the invention after incubation at various temperatures. c) shows mass spec analysis of different concentrations of a digested Proteome Standard Set of the invention after incubation at various temperatures.

### DETAILED DESCRIPTION OF THE INVENTION

### Definitions

In the description that follows, a number of terms used in recombinant DNA technology and protein chemistry are utilized extensively. In order to provide a clear and consistent understanding of the specification and claims, including the scope to be given such terms, the following definitions are provided.

As used herein, the articles “a,” “an” and “one” mean “at least one” or “one or more” of the object to which they refer, unless otherwise specified or made clear by the context in which they appear herein.

As used herein, the terms “about” or “approximately” when referring to any numerical value are intended to mean a value of ±10% of the stated value. For example, “about 50° C.” (or “approximately 50° C.”) encompasses a range of temperatures from 45° C. to 55° C., inclusive. Similarly, “about 100 mM” (or “approximately 100 mM”) encompasses a range of concentrations from 90 mM to 110 mM, inclusive.

As used herein “native” means nondenaturing or nondenatured, and refers to 1) conditions that do not disrupt intermolecular interactions within peptides or proteins that allow them to maintain a three dimensional structure that is either a three dimensional structure of the protein as found in nature or synthesized in a cell-free in vitro translation system, or 2) to proteins having a three dimensional structure that is the same or substantially the same as a three dimensional structure of the protein as found in nature or synthesized in a cell-free in vitro translation system. A three dimensional structure can be a secondary, tertiary, or quaternary structure of a protein.

The term “label” as used herein refers to a chemical moiety or protein that is directly or indirectly detectable (e.g. due to its spectral properties, conformation or activity) when attached to a target or compound and used in the present methods. The label can be directly detectable (fluorophore, chromophore) or indirectly detectable (hapten or enzyme). Such labels include, but are not limited to, radiolabels that can be measured with radiation-counting devices; pigments, dyes or other chromophores that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, dyes, fluorophores, haptens, enzymes and their calorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9^{th} edition, CD-ROM, September 2002), supra.

The term “directly detectable” as used herein refers to the presence of a material or the signal generated from the material is immediately detectable by observation, instrumentation, or film without requiring chemical modifications or additional substances.

A “dye” is a visually detectable label. A dye can be, for example, a chromophore or a fluorophore. A fluorophore can be excited by visible light or non-visible light (for example, UV light).

“Amino acid” refers to the twenty naturally-occurring amino acids, as well as to derivatives of these amino acids that occur in nature or are produced outside of living organisms by chemical or enzymatic derivatization or synthesis (for example, hydroxyproline, selenomethionine, azido amino acids, etc.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having acidic side chains is glutamic acid and aspartic acid; a group of amino acids having amino-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. Preferred conservative amino acid substitution groups are: valine-leucine-isoleucine; phenylalanine-tyrosine; lysine-arginine; alanine-valine; glutamic acid-aspartic acid; and asparagine-glutamine.

As used herein, “protein” means a polypeptide, or a sequence of two or more amino acids, which can be naturally-occurring or synthetic (modified amino acids, or amino acids not known in nature) linked by peptide bonds. “Peptide” specifically refers to polypeptides of less than 10 kDa. As used herein, the term “protein” encompasses peptides. In the context of the present invention, the term “protein” can refer to a multisubunit protein complex.

“Naturally-occurring” refers to the fact that an object having the same composition can be found in nature. For example, a polypeptide or polynucleotide sequence that is present in an organism, including viruses, that can be isolated from a source in nature, and that has not been intentionally modified in the laboratory is naturally-occurring.

A nucleic acid (or nucleotide) or protein (or amino acid) sequence that is “derived from” another nucleic acid (or nucleotide) or protein (or amino acid) sequence is either the same as at least a portion of the sequence it is derived from, or highly homologous to at least a portion of the sequence it is derived from. An amino acid sequence derived from the sequence of a naturally-occurring protein can be referred to as a “naturally-occurring protein-derived amino acid sequence”. A nucleic acid sequence derived from the sequence of a naturally-occurring nucleic acid can be referred to as a “naturally-occurring nucleic acid-derived nucleic acid sequence”. “Highly homologous” in this context means that the sequence is at least 80% identical at the amino acid level, preferably 90% identical at the amino acid level, and more preferably is at least 95% identical at the amino acid level. In the context of protein standards of the invention, two nucleic acid sequences are “homologous” when they are at least 65% identical, preferably at least 70% identical, and are highly homologous when they are at least 80% identical, and more preferably at least 90% identical.

“Recombinant methods” are methods that include the manufacture of or use of recombinant nucleic acids (nucleic acids that have been recombined to generate nucleic acid molecules that are structurally different from the analogous nucleic acid molecule(s) found in nature). Recombinant methods can employ, for example, restriction enzymes, exonucleases, endonucleases, polymerases, ligases, recombination enzymes, methylases, kinases, phosphatases, topoisomerases, etc. to generate chimeric nucleic acid molecules, generate nucleotide sequence changes, or add or delete nucleic acids to a nucleic acid sequence. Recombinant methods include methods that combine a nucleic acid molecule directly or indirectly isolated from an organism with one or more nucleic acid sequences from another source. The sequences from another source can be any nucleic acid sequences, for example, gene expression control sequences (for example, promoter sequences, transcriptional enhancer sequences, sequence that bind inducers or promoters of transcription, transcription termination sequences, translational regulation sequences, internal ribosome entry sites (IRES's), splice sites, poly A addition sequences, poly A sequences, etc.), a vector, protein-encoding sequences, etc. The nucleic acid sequences from a source other than the source of the nucleic acid molecule directly or indirectly isolated from an organism can be nucleic acid sequences from or within the genome of a different organism. Nucleic acid sequences in the genome can be chromosomal or extra-chromosomal (for example, the nucleic acid sequences can be episomal or of an organelle genome). Recombinant methods also includes methods of introducing nucleic acids into cells, including transformation, viral transfection, etc. to establish recombinant nucleic acid molecules in cells. “Recombinant methods” also includes the synthesis and isolation of products of nucleic acid constructs, such as recombinant RNA molecules and recombinant proteins. “Recombinant methods” is used interchangeably with “genetic engineering” and “recombinant [DNA] technology”.

A “recombinant protein” is a protein made from a recombinant nucleic acid molecule or construct. A recombinant protein can be made in cells harboring a recombinant nucleic acid construct, which can be cells of an organism or cultured prokaryotic or eukaryotic cells, or can made in vitro using, for example, in vitro transcription and/or translation systems.

“Do not differ substantially” or “substantially the same” means that the referenced compositions or components differ by less than 10% of the larger of the compared values.

The term “purified” as used herein refers to a preparation of a protein that is essentially free from contaminating proteins that normally would be present in association with the protein, e.g., in a cellular mixture or milieu in which the protein or complex is found endogenously such as serum proteins or cellular lysate.

“Substantially purified” refers to the state of a species or activity that is the predominant species or activity present (for example on a molar basis it is more abundant than any other individual species or activities in the composition) and preferably a substantially purified fraction is a composition in which the object species or activity comprises at least about 50 percent (on a molar, weight or activity basis) of all macromolecules or activities present. Generally, a substantially pure composition will comprise more than about 80 percent of all macromolecular species or activities present in a composition, more preferably more than about 85%, 90%, or 95%.

The term “sample” as used herein refers to any material that may contain a biomolecule or an analyte for detection or quantification.

The term “peptide segment” refers to a linear sequence of amino acids bordered on at least one side by a protease cleavage site. The linear sequence of amino acids may have, for example, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 120, 140, 160, 180, 200, 250, 300, 350, 400, 450, or 500 amino acids.

By “bordered by a protease cleavage site” is meant that at least one end of a peptide, either the carboxy terminal end or the amino terminal end, has an amino acid sequence that would be the sequence remaining following protease cleavage, by, for example, but not limited to, trypsin. Where a polypeptide comprises a segment bordered by a protease cleavage site, in the intact polypeptide the segment has, at least one end, a protease cleavage site. An internal segment would, for example, have a protease cleavage site at each end.

By “unique” is meant that a protein, a polypeptide, a peptide, or a peptide segment has an amino acid sequence that is not identical to any of the other proteins, polypeptides, peptides, or peptide segments in the standard set.

By “plurality” is meant at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 120, 140, 160, 180, 200, 250, 300, 350, 400, 450, or 500. By “plurality” is also meant at least 20-30, 20-40, 20-50, 20-75, 20-100, 20-150, 20-200, 25-30, 25-40, 25-50, 25-75, 25-100, 25-150, 25-200, 30-40, 30-50, 30-75, 30-100, 30-150, 30-200, 35-40, 35-50, 35-75, 35-100, 35-150, 35-200, 40-50, 40-75, 40-100, 40-150, 40-200, 45-50, 45-75, 45-100, 45-150, 45-200, 50-75, 50-100, 50-150, or 50-200.

Where a protein is “from a single species of organism,” such as, for example, human, the protein has the same sequence as the corresponding protein of that species, or has at least 85%, 90, 95, 97, or 98% homology to the amino acid sequence of that species' protein, or the protein was isolated from that species. That is, the proteins may be synthesized using recombinant DNA technology, or other means of synthesis.

A “customer” refers to any individual, institution, or business entity, such as a corporation, university, or organization, including a government entity or organization seeking to obtain genomic and proteomic products and services. A customer typically provides consideration, typically by paying money to a provider for a product or a service.

A “provider” refers to any individual, institution, business entity such as a corporation, university, or organization, including a government entity or organization, seeking to provide genomic and proteomic products and services. A provider typically receives consideration, typically monetary consideration, for providing a product or service to a customer. A provider typically provides a product or service in commerce to be sold and, with respect to products, shipped, either directly or indirectly to a customer.

A “commercial product” is a product that is sold and/or shipped through a stream of commerce. For example, a commercial product is typically sold and shipped, either directly, or indirectly using a third party, by a provider to a customer.

A “certifying authority” is a person or organization responsible for reviewing the results of analysis of the standard set, and comparing the results to a reference list of proteins, polypeptides, or peptides present in the standard set. A certifying authority may be a governmental institution or other institution, may be a person or office in a company or institution, such as an office in a university. A group of collaborating institutions or laboratories may designate a person or office to be a certifying authority, responsible for standardizing the laboratory techniques and equipment of all of the participants in the collaboration.

### Proteome Standard Set

The present invention provides proteome standard sets and methods of selecting and preparing proteome standard sets. The proteome standard sets replicate properties of a proteome, such as the proteome of a particular species or tissue, in particular properties, such as, for example, molecular weight of the protein standards, molecular weights of peptides generated by proteolysis of the protein standards, isoelectric point of the protein standards, isoelectric point of peptides generated by proteolysis of the protein standards, hydrophobicity of the protein standards, or hydrophobicity of peptides generated by proteolysis of the protein standards. The proteome standards replicate one or more of these or other properties by having a smaller number of proteins in the set than are present in the genome, in which the same or similar proportions of proteins in the standard set have the properties of interest as found in the proteome of interest.

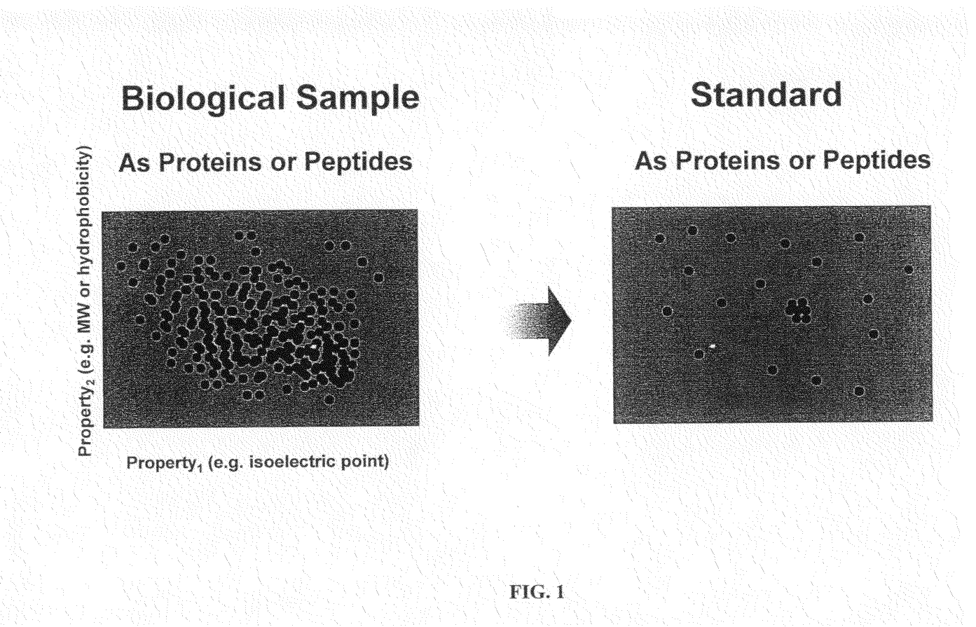

For example, as illustrated in FIG. 1, a particular biological sample may have a proteome in which the component proteins or generated peptides of the proteins distribute with respect to a particular property (Property 1), for example, isolelectric point, and a second property (Property 2), such as, for example, hydrophobicity. A subset of proteins can be used for a standard set that has fewer proteins than the biological sample, but exhibits the same range of these properties, and, potentially, the same or a similar distribution of particular properties, such as a clustering of 2, 3, 4, 5, or more proteins or peptides within a certain narrow pI range and certain narrow hydrophobicity range (as illustrated in the Standard panel on the right of FIG. 1).

Criteria can be established for a Proteome Standard set that represents the range and distribution of particular properties of proteins of a given proteome or peptides generated from proteins of a given proteome. The standards then can be used to ensure that the separation and/or analysis techniques used by a laboratory, technician, or performed by one or more pieces of laboratory equipment are able to separate or analyze a sample adequately. In some embodiments, a Proteome Standard set is used to ensure that proteins of a sample can be identified correctly. In some embodiments, a Proteome Standard set is used to ensure that proteins of a sample can be identified correctly using mass spectrometry. In some exemplary embodiments, the Proteome Standard set is designed so that a range of protein molecular weights is represented in the set, and a range or clustering of peptides generated by proteolyzing the proteins of the protein standard set is represented. For example, proteins of molecular weights ranging from, for example, 5 kDa to 500 kDa, or 10 kDa to 250 kDa, or 15 kDa to 200 kDa, or 20 kDa to 150 kDa, or 30 kDa to 125 kDa, or 32 kDa to 115 kDa can be present in the Proteome Standard Set, while peptides resulting from protease digestion of the set can range from about 1 Da to about 20 Da or more, with a certain number or percentage of the peptides falling within one or more particular molecular weight ranges. In this way, a Proteome Standard set can replicate a proteome, such as the human proteome, in which peptides generated by trypsin digestion of proteins results in a large number of peptides with similar or nearly identical (within 10, within 8, within 5, within 4, within 3, within 2, within 1, or within 0.5 Da) molecular weights.

For example, criteria can be developed in which a set comprises from 5 to 100 polypeptides that when proteolytically cleaved generate one, two three, four, five, or more clusters of peptides falling within a molecular weight range. For example, a cluster can be a molecular weight range of between 800 and 810 Da, between 990 and 1000 Da, between 1200 and 1210 Da, between 1500 and 1505 Da, as nonlimiting examples.

In exemplary embodiments, the standard sets comprise human proteins, with little contamination, for example, less than 10%, 5%, 4%, 3%, 2%, 1%, contamination by non-human proteins, such as, for example, E. coli proteins. The standards may be used, for example for cross-site comparisons of laboratory techniques and equipment, as standards for protocol development and for certifying laboratories, equipment, and laboratory technicians. The standards may be used, for example, to assess the capabilities of laboratories, equipment, and laboratory technicians to identify proteins, for example human proteins, to quantitate the amount of one or more individual proteins present in the set, and to assess sensitivity of the protocols used. The standards may be used for protein analysis protocols, including, for example, mass spectrometry and 2-D gel electrophoresis.

The proteome standards of a proteome standard set of the invention are, in exemplary embodiments, proteins of a single species, in which when the proteins are proteolyzed with a single or multiple proteolytic agents (e.g., proteases, cyanogens bromide, etc.) the proteins in aggregate give rise to multiple fragments that differ from one another by 10 Da or less. Preferably, the proteome standard set comprises 3 or more proteins that give rise to 3 or more proteolytic fragments that differ from one another by 10 Da or less. The proteome standard set in some exemplary embodiments comprises 3, 4, 5, 6, 7, 8, 9, 10, 11, 12, 13, 14, 15, 16, 17, 18, 19, 20, 25, 20, 35, 40, 45, 50, 55, 60, 65, 70 or more proteins that, in aggregate give rise to at least 3, at least 4, at least 5 at least 6 at least 7 at least 8 at least 9 at least 10 at least 11 at least 12, at least 13, at least 14, at least 15, at least 16, at least 17, at least 18, at least 19, at least 20 peptides that differ from one another by 10 Da or less. The proteome standard set in some exemplary embodiments comprises 3, 4, 5, 6, 7, 8, 9, 10, 11, 12, 13, 14, 15, 16, 17, 18, 19, 20 or more proteins that, when digested with the same proteolytic agent, each give rise to a peptide that is within 10 Da of a peptide produced by each of the other proteins of the proteome standard set.

Provided in the present invention are standard sets that include equimolar amounts of each protein of the set. For example, the concentration or amount of proteins in the set can differ by less than 5%, less than 3%, less than 2%, less than 1%, less than 0.5%, less than 0.2%, or less than 0.1%. Also provided are relative abundance standard sets where the proteins are not present in equimolar amounts. For example, within each molecular weight range, the proteins can have a difference in abundance of from about 5% to about 10%, of from about 10% to about 20%, or of from about 20% to about 50%, or of from about 50% to about 100%. The proteins can differ in abundance by 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 fold or more. Also provided are sets of relative abundance standard sets, for example, a set of two, three, or four different relative abundance standard sets, where each molecular weight range has a 5, 10, 20, 50, or 100 fold variation in protein abundance.

The invention also includes a method for mass spectrometry analysis that includes digesting a Proteome standard set comprising a plurality of proteins with one or more proteases to generate a set of proteolytic fragments derived from the standard set. The method further includes analyzing the set of proteolytic fragments by mass spectrometry, wherein no two proteolytic fragments between 700 to 4800 Da of the set of proteolytic fragments, are identical in amino acid sequence and at least five proteolytic fragments of the set of proteolytic fragments, produce mass peaks that differ from one another by less than 10 Da. Embodiments of this method can include digesting any of the standard set embodiments provided herein. For example, the plurality of proteins in the standard set can include at least 20 proteins of a single species of organism.

### Mass Spectrometry

In various aspects, the invention is drawn to mass spectroscopy. As used herein, the term “mass spectrometry” (or simply “MS”) encompasses any spectrometric technique or process in which molecules are ionized and separated and/or analyzed based on their respective molecular weights. Thus, as used herein, the terms “mass spectrometry” and “MS” encompass any type of ionization method, including without limitation electrospray ionization (ESI), atmospheric-pressure chemical ionization (APCI) and other forms of atmospheric pressure ionization (API), and laser irradiation.

Mass spectrometers are commonly combined with separation methods such as gas chromatography (GC) and liquid chromatography (LC). GC or LC separates the components in a mixture, and the components are then individually introduced into the mass spectrometer; such techniques are generally called GC/MS and LC/MS, respectively. MS/MS is an analogous technique where the first-stage separation device is another mass spectrometer. In LC/MS/MS, the separation methods comprise liquid chromatography and MS. Any combination (e.g., GC/MS/MS, GC/LC/MS, GC/LC/MS/MS, etc.) of methods can be used to practice the invention. In such combinations, “MS” can refer to any form of mass spectrometry; by way of non-limiting example, “LC/MS” encompasses LC/ESI MS and LC/MALDI-TOF MS. Thus, as used herein, the terms “mass spectrometry” and “MS” include without limitation APCI MS; ESI MS; GC MS; MALDI-TOF MS; LC/MS combinations; LC/MS/MS combinations; MS/MS combinations; etc.

### HPLC and RP-HPLC

It is often necessary to prepare samples comprising an analyte of interest for MS. Such preparations include without limitation purification and/or buffer exchange. Any appropriate method, or combination of methods, can be used to prepare samples for MS. One preferred type of MS preparative method is liquid chromatography (LC), including without limitation HPLC and RP-HPLC.

High-pressure liquid chromatography (HPLC) is a separative and quantitative analytical tool that is generally robust, reliable and flexible. Reverse-phase (RP) is a commonly used stationary phase that is characterized by alkyl chains of specific length immobilized to a silica bead support. RP-HPLC is suitable for the separation and analysis of various types of compounds including without limitation biomolecules, (e.g., glycoconjugates, proteins, peptides, and nucleic acids, and, with mobile phase supplements, oligonucleotides). One of the most important reasons that RP-HPLC has been the technique of choice amongst all HPLC techniques is its compatibility with electrospray ionization (ESI). During ESI, liquid samples can be introduced into a mass spectrometer by a process that creates multiple charged ions (Wilm et al., Anal. Chem. 68:1, 1996). However, multiple ions can result in complex spectra and reduced sensitivity.

In HPLC, peptides and proteins are injected into a column, typically silica based C18. An aqueous buffer is used to elute the salts, while the peptides and proteins are eluted with a mixture of aqueous solvent (water) and organic solvent (acetonitrile, methanol, propanol). The aqueous phase is generally HPLC grade water with 0.1% acid and the organic solvent phase is generally an HPLC grade acetonitrile or methanol with 0.1% acid. The acid is used to improve the chromatographic peak shape and to provide a source of protons in reverse phase LC/MS. The acids most commonly used are formic acid, trifluoroacetic acid, and acetic acid. In RP HPLC, compounds are separated based on their hydrophobic character. With an LC system coupled to the mass spectrometer through an ESI source and the ability to perform data-dependant scanning, it is now possible in at least some instances to distinguish proteins in complex mixtures containing more than 50 components without first purifying each protein to homogeneity. Where the complexity of the mixture is very high, it is possible to couple ion exchange chromatography and RP-HPLC in tandem to identify proteins from mixtures containing in excess of 1,000 proteins.

### MALDI-TOF MS

A particular type of MS technique, matrix-assisted laser desorption time-of-flight mass spectrometry (MALDI-TOF MS) (Karas et al., Int. J. Mass Spectrom. Ion Processes 78:53, 1987), has received prominence in analysis of biological polymers for its desirable characteristics, such as relative ease of sample preparation, predominance of singly charged ions in mass spectra, sensitivity and high speed. MALDI-TOF MS is a technique in which a UV-light absorbing matrix and a molecule of interest (analyte) are mixed and co-precipitated, thus forming analyte:matrix crystals. The crystals are irradiated by a nanosecond laser pulse. Most of the laser energy is absorbed by the matrix, which prevents unwanted fragmentation of the biomolecule. Nevertheless, matrix molecules transfer their energy to analyte molecules, causing them to vaporize and ionize. The ionized molecules are accelerated in an electric field and enter the flight tube. During their flight in this tube, different molecules are separated according to their mass to charge (m/z) ratio and reach the detector at different times. Each molecule yields a distinct signal. The method is used for detection and characterization of biomolecules, such as proteins, peptides, oligosaccharides and oligonucleotides, with molecular masses between about 400 and about 500,000 Da, or higher. MALDI-MS is a sensitive technique that allows the detection of low (10⁻¹⁵ to 10⁻¹⁸ mole) quantities of analyte in a sample.

Partial amino acid sequences of proteins can be determined by enzymatic proteolysis followed by MS analysis of the product peptides. These amino acid sequences can be used for in silico examination of DNA and/or protein sequence databases. Matched amino acid sequences can indicate proteins, domains and/or motifs having a known function and/or tertiary structure. For example, amino acid sequences from an uncharacterized protein might match the sequence or structure of a domain or motif that binds a ligand. As another example, the amino acid sequences can be used in vitro as antigens to generate antibodies to the protein and other related proteins from other biological source material (e.g., from a different tissue or organ, or from another species). There are many additional uses for MS, particularly MALDI-TOF MS, in the fields of genomics, proteomics and drug discovery. For a general review of the use of MALDI-TOF MS in proteomics and genomics, see Bonk et al. (Neuroscientist 7:12, 2001).

Tryptic peptides can be directly analyzed using MALDI-TOF. However, where sample complexity is apparent, on-line or off-line LC-MS/MS or two-dimensional LC-MS/MS may be necessary to separate the peptides. For example, for simple digests, a gradient of 5-45% (v/v) acetonitrile in 0.1% formic acid (or TFA, if MALDI MS/MS is available) over 45 min, and then 45-95% acetonitrile in 0.1% formic acid (or TFA, if MALDI MS/MS is available) over 5 min can be used. 0.1% Formic acid solution is used on the Q-TOF instrument and 0.1% TFA solution is used on the Dionex Probot fraction collector for off-line coupling between HPLC and MALDI-MS/MS analysis (carried out on the ABI 4700). For a complex sample, a gradient of 5-45% (v/v) acetonitrile over 90 min, and then 45-95% acetonitrile over 30 min can be used. For a very complex sample, a gradient of 5-45% (v/v) acetonitrile over 120 min, and then 45-95% acetonitrile over 60 min might be used. On the Q-TOF, one survey scan and four MS/MS data channels are used to acquire CID data with 1.4 s scan time.

Software programs such as MSQuant can be used for quantification of peptides and proteins (msquant.sourceforge.net).

### Kits

Kits including a protein standard set can be provided in which the proteins are in lyophilized or liquid form, in a container, such as, for example, a vial. The kit components are stable for 1, 2, 3, 4, 5, 6, 7, 8, 9, 10, 11, or 12 months at room temperature.

The invention provides in certain embodiments a kit comprising at least one container containing a proteome standard set. The proteome standard set may be an equimolar standard set or a relative abundance standard set.

The kits can further include at least one protein purification, isolation, or preparation reagent or at least one gel reagent, such as, for example, a sample or protein solubilizing buffer, a nondenaturing detergent (for example, dodecylmaltoside, octylglucoside, digitonin), gel loading buffer, an electrophoresis running buffer, a pre-cast native gel, or a gel stain, the kit may include proteolytic digestion reagents, such as, for example, trypsin. The kit can also include an instruction sheet that contains information on the analysis of the protein standards, and instructions on how to compare the analysis results with the reference results, either by sending the analysis results to a certifying authority, or by obtaining a set of reference results. Alternatively, the instruction sheet can refer the user to a web site that provides instructions.

### Methods of Generating Revenue

In certain aspects of the invention, a standard set of the present invention is provided that is a commercial product that is sold through interstate commerce using an instrument of commerce. The commercial product may be, for example, sold with a label and/or in a kit. The standard set is offered for sale by a provider, such as a for-profit business entity, to a customer. The commercial product may be provided, for example, as a liquid, or as a lyophilized powder. The liquid solution(s) can be shipped to a user in frozen or non-frozen liquid form.

In another embodiment, the method includes providing a means to purchase the standard set or a kit that includes the standard set. The method can further include activating the means to purchase the standard set and entering payment information. Furthermore, the method can include payment from a customer to a provider of the standard set.

The means to purchase the standard set is a purchasing function that can include means used by biological research reagent companies to sell reagents and/or kits, especially those for biological markers or standards. The method can include a telephonic system and/or an computer-based system. As a non-limiting example, the method can include displaying a link to purchase the standard set or kit on an Internet page or other displayed page on a local or wide area network. In addition, or alternatively, the means can be a telephone or text message ordering system. Another means, can include a direct order placed via traditional mail or an order placed verbally in person, for example with a salesperson. The standard set can be stocked in a supply center, in which a customer can remove one or more containers containing the standard sets, and record the amount of product taken on a page, in a book or ledger, or using a computer that is part of the stock center or accessed via the customer's personal computer (PC). The removal of product and recording of the removal of product can be performed by the purchaser or by an employee stock center or supplier of the product. The recording of the removal of the product constitutes an agreement on the part of the customer to pay for the standard set. Regardless of the means, typically the customer uses the means to purchase the standard set.

To purchase the standard set the customer gives consideration to the provider. Money is usually the form of consideration for the purchase paid by the customer to the provider. In exchange for the consideration, the provider who is typically an outside vendor, ships the standard set to the customer, typically an end-user customer. In the case of a stock center, an outside vendor ships to a stock center, typically within a research institution or company, and the purchaser removes the standard set and subsequently pays for the purchase, typically after receiving a bill generated by the supplier from the product removal record. It will be understood that the customer can be any customer that expresses proteins. For example, the customer can be a researcher at a research entity such as a research institute or a commercial entity. The customer can also be a medical diagnostics or pharmaceutical company, or a researcher therein.

The standard set can cost, for example, between $1 and $500, for example, for one sample standard set comprising sufficient protein for one analysis.

The purchasing function can be used to purchase additional products that are directly or indirectly related to the standard set provided herein. For example, the purchasing function can further be used to purchase reagents used for protein separation or isolation, and reagents used for analysis, such as, for example, electrophoresis gels, buffers, molecular weight or pI markers, HPLC columns and buffers, or mass spectrometry standards.

The standard set is typically stable for at least one month at 4 degrees Centigrade, and in certain aspects is stable for at least 2, 3, 4, 5, 6, 7, 8, 9, 10, 11, or 12 months at 4 degrees Centigrade or −20 degrees Centigrade, up to between 2, 3, 4, 5, 6, 7, 8, 9, 10, 11, 12, 13, 14, 15, 16, 17, 18, and 24 months or longer at 4 degrees Centigrade or −20 degrees Centigrade.

The method can further include shipping the standard set. For example, the shipping can include shipping using interstate commerce. The shipping is typically done by a provider to a customer. The customer is typically not in the same building as the provider. The shipping typically performed by a commercial carrier or a governmental entity, such as the U.S. Postal Service.

Another embodiment provided herein is a method for generating revenue, comprising: providing a customer with a purchasing function to purchase a standard set, in which when the purchase function is used to purchase the standard set, revenue is generated by a provider of either or both the purchase function and/or the standard set.

In another embodiment, the present invention also provides a method for selling a standard set and/or kit for protein expression, provided herein, including: presenting to a customer an input function of a telephonic ordering system, and/or presenting to a customer a data entry field or selectable list of entries as part of a computer system, in which the standard set and/or kit is identified using the input function. Where the input function is part of a computer system, such as displayed on one or more pages of an Internet site, the customer is typically presented with an on-line purchasing function, such as an online shopping cart, in which the purchasing function is used by the customer to purchase the identified standard set, and/or kit. In one aspect, a plurality of identifiers are provided to a customer, each identifying a standard set, and/or a kit provided herein in different volumes, or along with a related product such as an expression vector. The method may further comprise activating the purchasing function to purchase the standard set and/or kit provided herein.

Preferably, the standard set is ordered and provided to the customer as a kit. The method of generating revenue can also include providing the customer with a web site through which the customer can order a standard set provided herein. The web site also can electronically record the transaction and generate an invoice and/or a receipt.

Included in the standard set may be a form used for listing the analysis results, including instructions on where to submit the form for review by a certifying authority. Or, included in the standard set may be an Internet address, or website where the user may submit the results of analysis for review by a certifying authority.

The following examples are intended to illustrate but not limit the invention.

### EXAMPLE 1

### Selection of Initial Protein Pool

An initial protein pool comprising human protein sequences was selected. From this initial protein pool, a set of proteins was selected for a proteome standard set. The initial protein pool was selected from human open reading frame sequences (ORFs). The ULTIMATE™ ORF clone Collection (available by catalog on the world wide web at Invitrogen.com, Invitrogen, Carlsbad, Calif.) was used as a source of human ORFs. This selection may be conducted using computer software and bioinformatics methods known to those of ordinary skill in the art, as applied to publicly available human genome sequences. An initial review of the ranges of molecular weights of human ORFs available in the collection is as follows:

| | |
|---|---|
| 32-26 kDA: | 1064 ORFs |
| 48-52 kDA: | 632 ORFs |
| 70-75 kDA: | 298 ORFs |
| 100-115 kDA: | 199 ORFs |

It was found that there were a large number of closely related proteins in the collection due to existing homologs in the human genome, and to splice variants. In filtering the collection to provide a protein pool, isoleucine and leucine were considered identical.

Protein selection criteria for a set of 96 human ORFs were as follows:

1. Availability in the ‘ULTIMATE™ ORF collection’ available through Invitrogen (Invitrogen.com; Carlsbad, Calif.). About 10,000 ORFs are available in this collection.

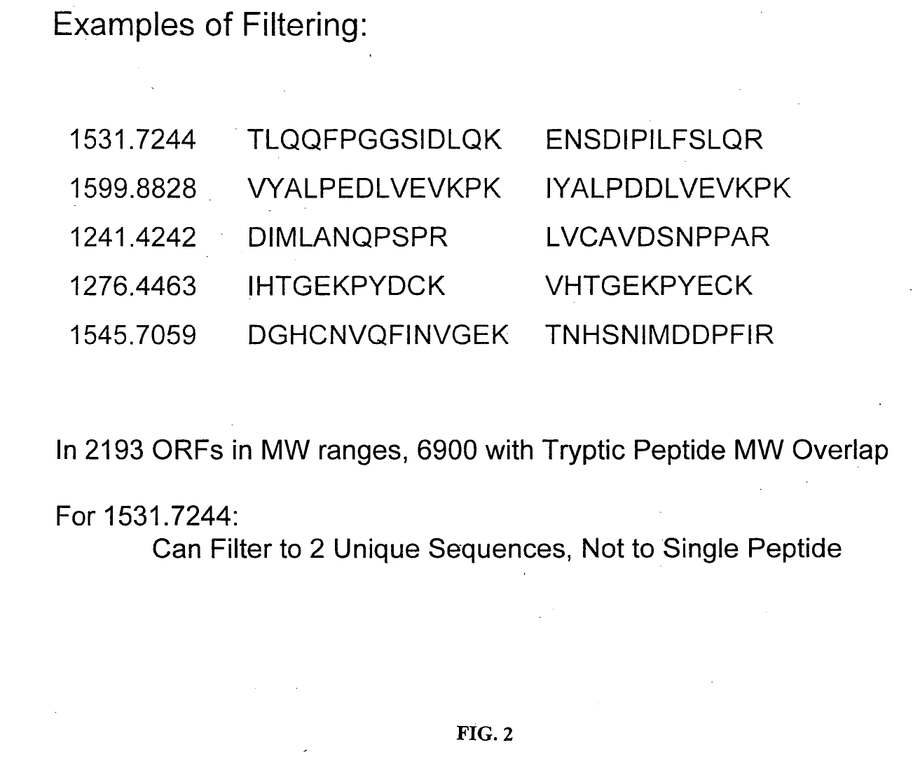

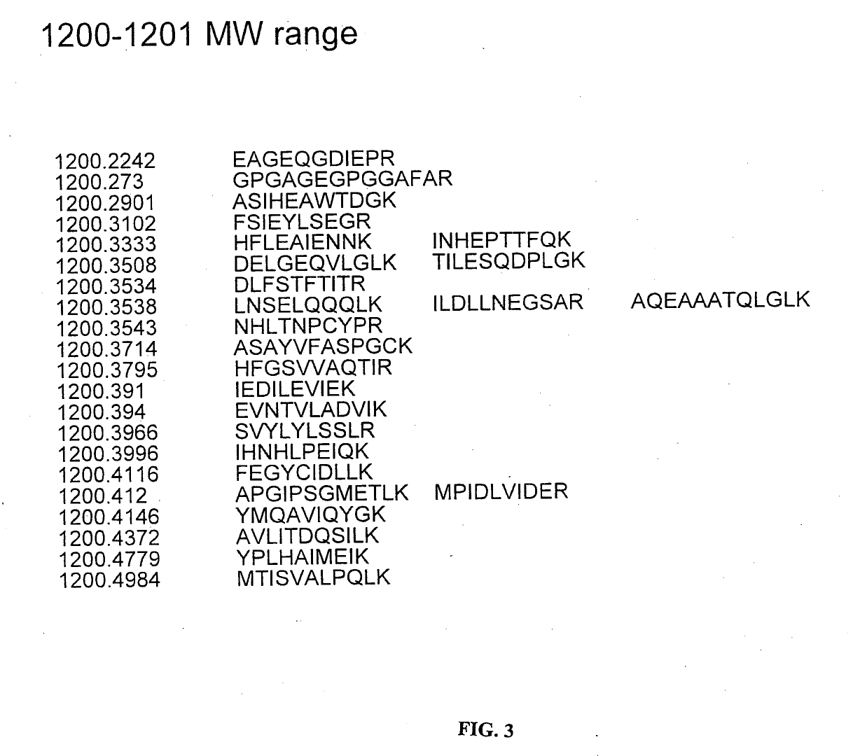

2. Proteins having unique tryptic peptide sequences (where isoleucine is considered equivalent to leucine) in the 700-4800 mass range. 7960 proteins of the collection met this criterion. In filtering the ORFs, multiple tryptic peptides had a molecular weight overlap, thus unique sequence was the criterion for inclusion in the pool. Examples of the molecular weight overlaps of peptides may be found in FIG. 2 and FIG. 3.

3. Proteins groups that met criterion 2 that contained greater than or equal to 96 members having multiple tryptic peptides of similar mass (e.g. 1202+/−0.5 atomic mass units (amu)). 550 proteins of the collection met this criterion.

4. 96 proteins of the collection (provided in Table 1) were selected based on populating the following protein molecular weight ranges: a. 32 ORFs: 33-36 kDab.16 ORFs: 50-52 kDac.16 ORFs: 70-74 kDad.32 ORFs: 101-114 kDa 5. The set of 96 proteins all contain tryptic peptides that fall within certain 10 Dalton molecular weight windows, such that a choice of any 20 proteins from the set will have 2 or more regions of the mass spectrum with 20 peptides within 10 Daltons of each other.

Isoelectric point (pI) has also been used to group the set of 96 proteins. It is possible that protein purification criteria may restrict the final set of 20 proteins to more narrow pI ranges than are present in the 96 protein set.

As final selection criteria, the following were used:

1. The translated ORF falls within one of four molecular weight ranges: 32-36 kDA, 48-52 kDA, 70-75 kDA, and 100-115 kDA

2. All tryptic peptides of the proteins (translated ORFs) have unique sequences.

3. Each translated ORF contains one or more peptides in the 1200-1210 range.

An initial protein pool of 96 human ORFs is presented in Table 1.

TABLE 1
Protein pool of 96 Human ORFs for selection of Proteome Standards

| Invitrogen | | | Genbank | |
|---|---|---|---|---|
| ULTIMATE ™ | | molecular | Accession | Genbank |
| ORF number | Protein Name | weight (daltons) | Number | identifier |
| IOH1960 | F-box protein 6 (FBXO6) | 33848.4692 | NM_018438 | 48995170 |
| | SEQ ID NO: 1 | | | |
| IOH3261 | interferon-related | 50128.4048 | BC001272 | 12654856 |
| | developmental regulator 1 | | | |
| | SEQ ID NO: 2 | | | |
| IOH3655 | methionyl-tRNA synthetase | 100905.4039 | NM_004990 | 14043021 |
| | (MARS) | | | |
| | SEQ ID NO: 3 | | | |
| IOH4374 | lon peptidase 1, mitochondrial | 106194.8507 | NM_004793 | 21396488 |
| | (LONP1) | | | |
| | SEQ ID NO: 4 | | | |
| IOH4401 | pyridoxal (pyridoxine, | 34976.1419 | NM_003681 | 148235211 |
| | vitamin B6) kinase (PDXK) | | | |
| | SEQ ID NO: 5 | | | |
| IOH4837 | kinesin family member 22 | 73093.8738 | NM_007317 | 6453817 |
| | (KIF22) | | | |
| | SEQ ID NO: 6 | | | |
| IOH5426 | replication factor C (activator | 35159.4512 | NM_002914 | 31563535 |
| | 1) 2, 40 kDa (RFC2), transcript | | | |
| | variant 2 | | | |
| | SEQ ID NO: 7 | | | |
| IOH6288 | ketohexokinase (fructokinase) | 32674.2259 | BC006233 | 33873491 |
| | SEQ ID NO: 8 | | | |
| IOH6982 | mitochondrial ribosomal | 33067.0125 | NM_016034 | 16554605 |
| | protein S2 (MRPS2) | | | |
| | SEQ ID NO: 9 | | | |
| IOH9666 | matrilin 2 | 104271.064 | BC010444 | 14714612 |
| | SEQ ID NO: 10 | | | |
| IOH9779 | inositol hexaphosphate kinase 1 | 49983.279 | NM_153273 | 58530860 |
| | (IHPK1) | | | |
| | SEQ ID NO: 11 | | | |
| IOH10054 | hydrolethalus syndrome 1 | 32844.0885 | BC015047 | 15929191 |
| | SEQ ID NO: 12 | | | |
| IOH10131 | CTTNBP2 N-terminal like | 69989.7052 | NM_018704 | 24308178 |
| | (CTTNBP2NL) | | | |
| | SEQ ID NO: 13 | | | |
| IOH10360 | solute carrier family 12 | 34673.2931 | BC020506 | 18088988 |
| | (potassium/chloride | | | |
| | transporters), member 8 | | | |
| | SEQ ID NO: 14 | | | |
| IOH10388 | general transcription factor | 100330.0941 | BC020981 | 18088104 |
| | IIIC, polypeptide 2, beta | | | |
| | 110 kDa | | | |
| | SEQ ID NO: 15 | | | |
| IOH10404 | glucosaminyl (N-acetyl) | 50639.3294 | NM_004751 | 4758421 |
| | transferase 3, mucin type | | | |
| | (GCNT3) | | | |
| | SEQ ID NO: 16 | | | |
| IOH10978 | CDP-diacylglycerol synthase | 51249.7334 | NM_003818 | 22035625 |
| | (phosphatidate | | | |
| | cytidylyltransferase) 2 (CDS2) | | | |
| | SEQ ID NO: 17 | | | |
| IOH11062 | spermatogenesis associated 4 | 34626.2371 | BC021731 | 34785039 |
| | SEQ ID NO: 18 | | | |
| IOH11193 | ecto-NOX disulfide-thiol | 73138.0125 | BC024178.1 | 18848203 |
| | exchanger 1 | | | |
| | SEQ ID NO: 19 | | | |
| IOH11418 | eukaryotic translation initiation | 50955.2721 | NM_001415.3 | 83656782 |
| | factor 2, subunit 3 gamma, | | | |
| | 52 kDa (EIF2S3) | | | |
| | SEQ ID NO: 20 | | | |
| IOH11514 | splicing factor, arginine/serine- | 51384.2228 | BC028151 | 20380085 |
| | rich 17A | | | |
| | SEQ ID NO: 21 | | | |
| IOH11833 | ankyrin repeat domain 9 | 34197.2789 | NM_152326.2 | 50355986 |
| | (ANKRD9) | | | |
| | SEQ ID NO: 22 | | | |
| IOH11958 | eukaryotic translation initiation | 104993.3357 | NM_003752 | 76443656 |
| | factor 3, subunit 8, 110 kDa | | | |
| | (EIF3S8), transcript variant 1 | | | |
| | SEQ ID NO: 23 | | | |
| IOH12255 | KIAA1199 | 109712.7198 | BC020256 | 18044272 |
| | SEQ ID NO: 24 | | | |
| IOH12674 | FERM, RhoGEF and pleckstrin | 72988.8316 | BC021301 | 233878572 |
| | domain protein | | | |
| | SEQ ID NO: 25 | | | |
| IOH12799 | chromosome 15 open reading | 34626.8224 | NM_024713 | 13376012 |
| | frame 29 (C15orf29) | | | |
| | SEQ ID NO: 26 | | | |
| IOH12840 | DiGeorge syndrome critical | 32775.2314 | BC009984 | 833874937 |
| | region gene | | | |
| | SEQ ID NO: 27 | | | |
| IOH13263 | ring finger protein 25 (RNF25) | 51022.4416 | NM_022453 | 34878786 |
| | SEQ ID NO: 28 | | | |
| IOH13463 | BCR downstream signaling 1 | 34235.2936 | NM_012108 | 6912271 |
| | (BRDG1) | | | |
| | SEQ ID NO: 29 | | | |
| IOH13536 | sprouty homolog 2 | 34548.1888 | NM_005842 | 22209007 |
| | (Drosophila) (SPRY2) | | | |
| | SEQ ID NO: 30 | | | |
| IOH13891 | DEAD (Asp-Glu-Ala-Asp) box | 73047.1248 | NM_001356 | 87196350 |
| | polypeptide 3, X-linked | | | |
| | (DDX3X) | | | |
| | SEQ ID NO: 31 | | | |
| IOH14101 | solute carrier family 25, | 34198.4304 | BC014064 | 15559392 |
| | member 36 | | | |
| | SEQ ID NO: 32 | | | |
| IOH14291 | translin-associated factor X | 33000.3302 | NM_005999 | 20302159 |
| | (TSNAX) | | | |
| | SEQ ID NO: 33 | | | |
| IOH14374 | synaptotagmin-like 1 | 50047.7652 | BC015764 | 16041766 |
| | SEQ ID NO: 34 | | | |
| IOH14568 | cell division cycle 16 homolog | 71333.2749 | NM_001078645 | 118402577 |
| | (S. cerevisiae) (CDC16), | | | |
| | transcript variant 2 | | | |
| | SEQ ID NO: 35 | | | |
| IOH14731 | Wolf-Hirschhorn syndrome | 72410.8185 | NM_017778 | 13699812 |
| | candidate 1-like 1 | | | |
| | (WHSC1L1), transcript variant | | | |
| | short | | | |
| | SEQ ID NO: 36 | | | |
| IOH14889 | phosphatidylinositol glycan | 33512.4135 | NM_002642 | 42519917 |
| | anchor biosynthesis, class C | | | |
| | (PIGC), transcript variant 2 | | | |
| | SEQ ID NO: 37 | | | |
| IOH20971 | protein tyrosine phosphatase, | 73581.9682 | NM_002849 | 119743915 |
| | receptor type, R (PTPRR), | | | |
| | transcript variant | | | |
| | SEQ ID NO: 38 | | | |
| IOH21022 | phosphoinositide-3-kinase, | 101297.0394 | NM_002647 | 34761063 |
| | class 3 (PIK3C3) | | | |
| | SEQ ID NO: 39 | | | |
| IOH21070 | protein tyrosine phosphatase, | 105504.5421 | NM_002830 | 18104987 |
| | non-receptor type 4 | | | |
| | (megakaryocyte) (PTPN4) | | | |
| | SEQ ID NO: 40 | | | |
| IOH21514 | chromosome 13 open reading | 33454.9646 | NM_152325.1 | 22748710 |
| | frame 26 (C13orf26) | | | |
| | SEQ ID NO: 41 | | | |
| IOH21701 | family with sequence similarity | 102812.2427 | NM_052966 | 93277091 |
| | 129, member A (FAM129A), | | | |
| | transcript variant 2 | | | |
| | SEQ ID NO: 42 | | | |
| IOH21708 | glycerol kinase 5 (putative) | 33818.0145 | BC032470.1 | 21595467 |
| | SEQ ID NO: 43 | | | |
| IOH21907 | glycine N-methyltransferase | 32602.1058 | NM_018960.4 | 54792737 |
| | (GNMT) | | | |
| | SEQ ID NO: 44 | | | |
| IOH22443 | eukaryotic translation initiation | 107850.4639 | BC033028.1 | 21542534 |
| | factor 4E nuclear import factor 1 | | | |
| | SEQ ID NO: 45 | | | |
| IOH23101 | ATP synthase mitochondrial F1 | 32688.283 | NM_145691 | 52426740 |
| | complex assembly factor 2 | | | |
| | (ATPAF2), nuclear gene | | | |
| | encoding mitochondrial protein | | | |
| | SEQ ID NO: 46 | | | |
| IOH23111 | MCF.2 cell line derived | 111497.1484 | NM_024979 | 27777654 |
| | transforming sequence-like | | | |
| | (MCF2L) | | | |
| | SEQ ID NO: 47 | | | |
| IOH23115 | centrobin, centrosomal BRCA2 | 100930.4135 | BC021134 | 21594795 |
| | interacting protein | | | |
| | SEQ ID NO: 48 | | | |
| IOH23248 | EF-hand calcium binding | 32674.9659 | NM_173584 | 150170652 |
| | domain 4A (EFCAB4A) | | | |
| | SEQ ID NO: 49 | | | |
| IOH25926 | chromosome 4 open reading | 50547.7852 | NM_174952 | 3345732\| |
| | frame 37 (C4orf37) | | | |
| | SEQ ID NO: 50 | | | |
| IOH25943 | zinc finger protein 462 | 100883.5498 | BC036884 | 22477431 |
| | SEQ ID NO: 51 | | | |
| IOH26161 | forty-two-three domain | 35804.2291 | NM_032288 | 58374126 |
| | containing 1 (FYTTD1), | | | |
| | transcript variant 1 | | | |
| | SEQ ID NO: 52 | | | |
| IOH26205 | HIR histone cell cycle | 111540.6687 | NM_003325 | 21536484 |
| | regulation defective homolog A | | | |
| | (S. cerevisiae) | | | |
| | (HIRA) SEQ ID NO: 53 | | | |
| IOH26344 | general transcription factor | 100950.7839 | BC043347 | 28175201 |
| | IIIC, polypeptide 3, 102 kDa | | | |
| | SEQ ID NO: 54 | | | |
| IOH26703 | esophageal cancer | 109198.1066 | NM_020314 | 142371913 |
| | associated protein | | | |
| | (MGC16824) | | | |
| | SEQ ID NO: 55 | | | |
| IOH26721 | thrombospondin 4 (THBS4) | 105700.9438 | NM_003248 | 40549419 |
| | SEQ ID NO: 56 | | | |
| IOH26735 | F-box protein 38 | 107268.8089 | BC050424 | 29792183 |
| | SEQ ID NO: 57 | | | |
| IOH26831 | WD repeat domain 47 | 101502.4827 | NM_014969 | 141802599 |
| | (WDR47) | | | |
| | SEQ ID NO: 58 | | | |
| IOH27002 | ribosomal protein SA | 32827.9848 | BC050688 | 30047124 |
| | SEQ ID NO: 59 | | | |
| IOH27156 | complement factor H | 50893.631 | BC037285 | 34190531 |
| | SEQ ID NO: 60 | | | |
| IOH27296 | START domain containing 3 | 50403.9702 | NM_006804 | 31543656 |
| | (STARD3) | | | |
| | SEQ ID NO: 61 | | | |
| IOH27512 | WD repeat domain 66 | 106593.2717 | BC036233 | 23271966 |
| | SEQ ID NO: 62 | | | |
| IOH27680 | zinc finger CCCH-type | 101522.8325 | NM_015117 | 30794493 |
| | containing 3 (ZC3H3) | | | |
| | SEQ ID NO: 63 | | | |
| IOH27788 | tRNA-histidine | 34702.6624 | BC023521 | 39644525 |
| | guanylyltransferase 1-like (S. cerevisiae) | | | |
| | SEQ ID NO: 64 | | | |
| IOH27961 | growth arrest-specific 2 | 34861.3256 | NM_005256 | 29540560 |
| | (GAS2), transcript variant 1 | | | |
| | SEQ ID NO: 65 | | | |
| IOH28456 | olfactory receptor, family 9, | 34851.6338 | NM_001005211 | 52627200 |
| | subfamily I, member 1 (OR9I1) | | | |
| | SEQ ID NO: 66 | | | |
| IOH28733 | SUMO1/sentrin specific | 73030.1643 | BC045639 | 28279001 |
| | peptidase 1 | | | |
| | SEQ ID NO: 67 | | | |
| IOH28743 | UDP-N-acetyl-alpha-D- | 70850.2268 | NM_007210 | 115298683 |
| | galactosamine:polypeptide N- | | | |
| | acetylgalactosaminyltransferase | | | |
| | 6 (GalNAc-T6) (GALNT6) | | | |
| | SEQ ID NO: 68 | | | |
| IOH28815 | guanylate cyclase 1, soluble, | 70248.1549 | NM_000857 | 4504214 |
| | beta 3 (GUCY1B3) | | | |
| | SEQ ID NO: 69 | | | |
| IOH28842 | RAB GTPase activating protein | 113890.2024 | NM_012197 | 12232372 |
| | 1 (RABGAP1) | | | |
| | SEQ ID NO: 70 | | | |
| IOH28918 | myotubularin related protein 2 | 73101.8274 | BC052990 | 31418326 |
| | SEQ ID NO: 71 | | | |
| IOH29030 | thymopoietin (TMPO), | 50600.0793 | NM_001032283 | 73760404 |
| | transcript variant 2 | | | |
| | SEQ ID NO: 72 | | | |
| IOH29038 | CREB regulated transcription | 72937.4204 | NM_181715 | 32171214 |
| | coactivator 2 (CRTC2) | | | |
| | SEQ ID NO: 73 | | | |
| IOH29053 | leucine zipper, putative tumor | 72619.1373 | BC058938 | 37589138 |
| | suppressor 2 | | | |
| | SEQ ID NO: 74 | | | |
| IOH29116 | plasmalemma vesicle | 50524.1117 | NM_031310 | 13775237 |
| | associated protein (PLVAP) | | | |
| | SEQ ID NO: 75 | | | |
| IOH29199 | KIAA0746 protein | 111221.4517 | NM_015187 | 142379857 |
| | (KIAA0746) | | | |
| | SEQ ID NO: 76 | | | |
| IOH29386 | coagulation factor II (thrombin) | 69826.5331 | BC051332 | 30802114 |
| | SEQ ID NO: 77 | | | |
| IOH34752 | 5-hydroxytryptamine | 50137.5715 | NM_006028 | 47519847 |
| | (serotonin) receptor 3B | | | |
| | (HTR3B) | | | |
| | SEQ ID NO: 78 | | | |
| IOH35514 | Bartter syndrome, infantile, | 35127.0463 | NM_057176 | 20357592 |
| | with sensorineural deafness | | | |
| | (Barttin) (BSND) | | | |
| | SEQ ID NO: 79 | | | |
| IOH36706 | cysteine rich transmembrane | 113457.6886 | NM_016441 | 10092638 |
| | BMP regulator 1 (chordin-like) | | | |
| | (CRIM1) | | | |
| | SEQ ID NO: 80 | | | |
| IOH38530 | optic atrophy 1 (autosomal | 111252.1561 | NM_015560 | 18860828 |
| | dominant) (OPA1), nuclear | | | |
| | gene encoding mitochondrial | | | |
| | protein, transcript variant 1 | | | |
| | SEQ ID NO: 81 | | | |
| IOH38551 | SLIT and NTRK-like family, | 108485.3844 | NM_014926 | 40217819 |
| | member 3 (SLITRK3) | | | |
| | SEQ ID NO: 82 | | | |
| IOH40625 | galactosidase, beta 1 like 3 | 35221.739 | BC011001 | 33877524 |
| | SEQ ID NO: 83 | | | |
| IOH40805 | nucleoporin 50 kDa (NUP50), | 50059.9856 | NM_007172 | 82659110 |
| | transcript variant 2 | | | |
| | SEQ ID NO: 84 | | | |
| IOH40838 | nucleoporin 210 kDa\| | 105431.4877 | BC067089 | 45595563 |
| | SEQ ID NO: 85 | | | |
| IOH40849 | golgi autoantigen, golgin | 111406.0492 | NM_004486 | 47078236 |
| | subfamily a, 2 (GOLGA2) | | | |
| | SEQ ID NO: 86 | | | |
| IOH40892 | kelch-like 13 (Drosophila) | 73643.6666 | NM_033495 | 45643137 |
| | (KLHL13) | | | |
| | SEQ ID NO: 87 | | | |
| IOH41756 | chromodomain helicase DNA | 100649.5619 | NM_004284 | 148612869 |
| | binding protein 1- | | | |
| | like(CHD1L) | | | |
| | SEQ ID NO: 88 | | | |
| IOH41791 | ligase I, DNA, ATP-dependent | 101567.7348 | NM_000234 | 4557718 |
| | (LIG1) | | | |
| | SEQ ID NO: 89 | | | |
| IOH42083 | SET domain containing 3 | 33631.1621 | NM_199123 | 40068482 |
| | (SETD3) | | | |
| | SEQ ID NO: 90 | | | |
| IOH42591 | pleiomorphic adenoma | 50412.8911 | NM_006718 | 124381122 |
| | gene-like 1 (PLAGL1), | | | |
| | transcript variant 2 | | | |
| | SEQ ID NO: 91 | | | |
| IOH43447 | KIAA1189 (KIAA1189), | 34174.6017 | NM_00100995 | 58331176 |
| | transcript variant 1 | | | |
| | SEQ ID NO: 92 | | | |
| IOH43981 | isoleucyl-tRNA synthetase 2, | 113469.0425 | NM_018060 | 46852146 |
| | mitochondrial (IARS2) | | | |
| | SEQ ID NO: 93 | | | |
| IOH44576 | glycine-N-acyltransferase | 33742.9575 | NM_201648 | 111038136 |
| | (GLYAT), nuclear gene | | | |
| | encoding mitochondrial | | | |
| | protein, transcript | | | |
| | variant 1 | | | |
| | SEQ ID NO: 94 | | | |
| IOH45987 | Sad1 and UNC84 domain | 34746.8798 | BC026189 | 34191786 |
| | containing 1 | | | |
| | SEQ ID NO: 95 | | | |
| IOH46182 | NIK and IKK{beta} binding | 100578.9357 | BC006206 | 34782806 |
| | protein | | | |
| | SEQ ID NO: 96 | | | |

### EXAMPLE 2

### Selection of a Proteome Standard Set

A proteome standard set comprising 20 proteins was selected from the initial protein pool using the following criteria:

1. Selected proteins range in molecular weight between 33-114 kDa.

2. Purity of each protein is such that an equimolar mixture of 20 proteins contains no individual contaminating protein at greater than 1% of the total mixture. Contamination of the sample is evaluated based on mass and on molar amount such that no contaminating protein is greater than 1% of the mass or molar amount of the total mixture.

3. Purity of an equimolar mixture of the 20 selected proteins is in the range of 95%-99% of the mixture. Purity is determined by the absence of contaminating non-human proteins, such as, for example, E. coli proteins.

### EXAMPLE 3

### Preparation of Equimolar Proteome Standard Sets and Use in Standardization

Proteins can be prepared using standard recombinant techniques, including expression using the vector pET-DEST42 (Invitrogen, Carlsbad, Calif.). In the purification procedures, inclusion body formation is maximized, inclusion bodies are purified, solubilized by denaturization, and the proteins purified under denatured conditions. Proteins may be purified using, for example, anion exchange chromatography. Reverse Phase chromatography in TFA/Acetonitrile is used as a final step in purification. This volatile buffer systems is more convenient for lyophilization.

A proteome standard set is prepared of 20 proteins mixed in equimolar amounts in a container. Each of the 20 proteins is present at 5 picomoles, for a total of 100 picomoles of protein. Characteristics of the standard set include the following:

1. The sample of 20 different proteins will have molecular weights between 33 kDa to 114 kDa.

2. The mixture will contain a minimum of 4 proteins in each of 4 different molecular weight ranges: a.33-36 kDab.50-53 kDac.70-75 kDad.100-115 kDa

3. An additional 4 proteins will be distributed amongst at least 2 of the 4 molecular weight ranges to make up the final 20 protein panel.

4. An equimolar mixture of the 20 proteins will be sent to each lab lyophilized.

5. All proteins will be 5 picomoles (+/−10% by amino acid analysis) total (in each vial).

The container is provided to a participating laboratory, or technician. A list of the proteins is provided to the certifying organization, or other person or institution responsible for assessing the laboratory or technician. The list is provided according to the criteria of Carr et al. 2004 Mol. Cell. Proteomics 3:531:533. The participating laboratory or technician uses standard laboratory techniques to separate the proteins, isolate the separated proteins, and analyze the isolated proteins by, for example, mass spectrometry or 2D gel electrophoresis, to identify the proteins. Or, the proteins are proteolytically cleaved after they are isolated, prior to analysis. The person or institution responsible for assessing the laboratory or technician then compares the results obtained by the analysis with the reference results on the list of proteins, or the expected proteolytic fragments that would be derived from the listed proteins.

### EXAMPLE 4

### Preparation of Relative Abundance Proteome Standard Sets and Use in Standardization

A proteome standard set is prepared of 20 proteins in non-equimolar amounts in a container. Different sample sets may be prepared, with each set having a different relative abundance from the other. In the present example, four samples (A, B, C, D) of different relative abundance are prepared. 25 μg of each sample of lyophilized proteins are present in each of 4 vials, each laboratory receives a total of 4 vials. The sample proteome standard sets are prepared as follows:

1. A sample of 20 different proteins, spanning a molecular weight range from 33 kDa to 114 kDa, will be mixed in 4 different ways, to produce mixtures with a maximum difference of 1000-fold between high and very low abundance proteins. Proteins will be distributed in 4 different MW ranges as described for the equimolar mix.

2. Four proteins will be at high abundance; 5 at medium; 8 at low abundance, 3 at very low abundance.

3. High, medium, low and very low abundance proteins will be distributed throughout the 4 different molecular weight ranges, such that at least 1 of the 4 molecular weight ranges contain proteins spanning the abundance range of each mix.

4. Proteins present at high abundance will be chosen to minimize non-human, contaminating proteins in the final mix. It is understood that contaminants of the high abundance proteins will be present in the final mixtures at higher molar amounts than the very low abundance human proteins.

5. Overall purity of the mixtures will be identical to that of the equimolar mixture, with no single contaminant being present at >1% and between 95%-99% of the mixture to consist of the 20 standard proteins.

The container is provided to a participating laboratory, or technician. A list of the proteins, and their relative abundance, is provided to the certifying organization, or other person or institution responsible for assessing the laboratory or technician. The list is provided according to the criteria of Carr et al. 2004 Mol. Cell. Proteomics 3:531:533. The participating laboratory or technician uses standard laboratory techniques to separate the proteins, isolate the separated proteins, and analyze the isolated proteins by, for example, mass spectrometry or 2D gel electrophoresis, to identify the proteins and their relative abundance. Or, the proteins are proteolytically cleaved after they are isolated, prior to analysis. The person or institution responsible for assessing the laboratory or technician then compares the results obtained by the analysis with the reference results on the list of proteins, or the expected proteolytic fragments that would be derived from the listed proteins.

Table 2 provides an example of possible protein proportions for a 20 protein relative abundance standard.

TABLE 2

| | | | | | Rel. | | | | |
|---|---|---|---|---|---|---|---|---|---|
| Protein | MW (kDa) | A (μg) | Rel. Ab. | B (μg) | Ab. | C (μg) | Rel. Ab. | D (μg) | Rel. Ab. |
| 1 | 114 | 0.0114 | 1 | 0.0228 | 2 | 0.057 | 5 | 0.0114 | 1 |
| 2 | 110 | 0.11 | 10 | 0.055 | 5 | 0.33 | 30 | 0.55 | 50 |
| 3 | 107 | 0.107 | 10 | 0.107 | 10 | 1.07 | 100 | 0.321 | 30 |
| 4 | 104 | 1.04 | 100 | 0.0104 | 1 | 0.208 | 20 | 0.832 | 80 |
| 5 | 101 | 0.101 | 10 | 0.505 | 50 | 0.303 | 30 | 0.0505 | 5 |
| 6 | 74 | 0.074 | 10 | 0.0074 | 1 | 0.0148 | 2 | 0.037 | 5 |
| 7 | 73 | 0.73 | 100 | 7.3 | 1000 | 0.365 | 50 | 0.073 | 10 |
| 8 | 72 | 0.0072 | 1 | 0.072 | 10 | 0.036 | 5 | 0.0144 | 2 |
| 9 | 70 | 0.7 | 100 | 0.35 | 50 | 1.75 | 250 | 3.5 | 500 |
| 10 | 52 | 0.052 | 10 | 0.104 | 20 | 0.26 | 50 | 0.52 | 100 |
| 11 | 51 | 5.1 | 1000 | 4.08 | 800 | 4.08 | 800 | 5.1 | 1000 |
| 12 | 51 | 0.051 | 10 | 0.051 | 10 | 0.051 | 10 | 0.051 | 10 |
| 13 | 50 | 0.5 | 100 | 1 | 200 | 0.25 | 50 | 0.01 | 2 |
| 14 | 36 | 0.0036 | 1 | 0.018 | 5 | 0.0072 | 2 | 0.0036 | 1 |
| 15 | 35.5 | 3.55 | 1000 | 0.355 | 100 | 0.142 | 40 | 0.0355 | 10 |
| 16 | 35 | 0.035 | 10 | 0.07 | 20 | 0.07 | 20 | 0.035 | 10 |
| 17 | 34.5 | 0.345 | 100 | 0.01725 | 5 | 1.725 | 500 | 0.1725 | 50 |
| 18 | 34 | 0.034 | 10 | 0.17 | 50 | 0.034 | 10 | 0.34 | 100 |
| 19 | 33.5 | 0.335 | 100 | 0.1675 | 50 | 1.675 | 500 | 0.8375 | 250 |
| 20 | 33 | 3.3 | 1000 | 0.66 | 200 | 2.64 | 800 | 0.33 | 100 |

| | | | | |
|---|---|---|---|---|
| Column Total | 16.1862 | 15.12235 | 15.068 | 12.8244 |

### EXAMPLE 5

### Use of Proteome Standard Sets in Standardization

In one example, a participating laboratory, or a technician, obtains the protein standard sets in lyophilized form, then dilutes the powder according to instructions provided, or methods known to those of ordinary skill in the art. A set of standards is subjected to gel electrophoresis, or liquid chromatography followed by gel electrophoresis. Bands of separated proteins were treated to in-gel tryptic digest, then the digested peptides were subjected to mass spectrometry.

Proteins may also be subjected to mass spectrometry by elution from the gel, with or without a proteolytic digestion step. Proteins may also be subjected to mass spectrometry after liquid chromatography, without a gel electrophoresis step. The gel electrophoresis step may also be 2-dimensional gel electrophoresis.

### EXAMPLE 6

### Proteome Standard Set with Equimolar Amounts of Proteins

Proteins were selected from the Ultimate™ Human ORF collection in order to simulate, with a small number of proteins, the complexity and diversity of actual biological samples, for example, in properties such as molecular weight, isoelectric point, and/or hydrophobicity (FIG. 1).

Biological samples display complexity and diversity in many dimensions (molecular weight, hydrophobicity, isoelectric point) at both the protein and peptide level. The selected protein standards are diverse at both the protein and peptide level, and the selection criteria ensure that clusters of complexity also exist in the standards at both the protein and peptide level. The more than 13,000 proteins in the ULTIMATE™ Human ORF clone collection were reduced to 2,000 by selecting only those proteins in four molecular weight “zones”. Selecting from these 2,000 a subset of proteins that produce tryptic peptides with unique sequences reduced the number of proteins to 1,500. The final filter selected proteins that all had one or more tryptic peptide(s) in the same 10 Da mass window; this reduced the number of candidate standard proteins to 250. Twenty human proteins of the Invitrogen Ultimate ORF collection (Invitrogen, Carlsbad, Calif.), were selected that ranged in molecular weight from 30 to 112 kDa. The identities of these proteins are provided in Tables 3 and 4.

TABLE 3
Proteins of 20 Protein Proteome Standard Set.

| | | Gene |
|---|---|---|
| Clone ID | Definition | Symbol |
| IOH6288 | ketohexokinase (fructokinase), transcript variant a, mRNA | KHK |
| IOH23101 | ATP synthase mitochondrial F1 complex assembly factor 2 (ATPAF2), | ATPAF2 |
| | nuclear gene encoding mitochondrial protein, mRNA | |
| IOH42083 | chromosome 14 open reading frame 154 (C14orf154), transcript variant 2, | C14orf154 |
| | mRNA | |
| IOH13536 | sprouty homolog 2 (Drosophila) (SPRY2), mRNA | SPRY2 |
| IOH40625 | hypothetical protein BC011001 (LOC112937), mRNA | LOC112937 |
| IOH26161 | forty-two-three domain containing 1 (FYTTD1), transcript variant 1, | FYTTD1 |
| | mRNA | |
| IOH9779 | inositol hexaphosphate kinase 1 (IHPK1), transcript variant 1, mRNA | IHPK1 |
| IOH3261 | interferon-related developmental regulator 1 (IFRD1), transcript variant 2, | IFRD1 |
| | mRNA | |
| IOH10404 | glucosaminyl (N-acetyl) transferase 3, mucin type (GCNT3), mRNA | GCNT3 |
| IOH11418 | eukaryotic translation initiation factor 2, subunit 3 gamma, 52 kDa | EIF2S3 |
| | (EIF2S3), mRNA | |
| IOH29386 | coagulation factor II (thrombin), mRNA | F2 |
| IOH12674 | FERM, RhoGEF and pleckstrin domain protein 2, mRNA | FARP2 |
| IOH11193 | hypothetical protein FLJ10094, mRNA | FLJ10094 |
| IOH40892 | kelch-like 13 (Drosophila) (KLHL13), mRNA | KLHL13 |
| IOH46182 | IKK2 binding protein, mRNA, complete cds. | NIBP |
| IOH3655 | methionine-tRNA synthetase (MARS), mRNA | MARS |
| IOH40838 | nucleoporin 210 kDa, mRNA | NUP210 |
| IOH26721 | thrombospondin 4 (THBS4), mRNA | THBS4 |
| IOH29199 | KIAA0746 protein (KIAA0746), mRNA | KIAA0746 |
| IOH26205 | HIR histone cell cycle regulation defective homolog A (S. cerevisiae) | HIRA |
| | (HIRA), mRNA | |

TABLE 4
Proteins of 20 Protein Proteome Standard Set

| | | | Protein | | Swiss- | | Theor. pl | |
|---|---|---|---|---|---|---|---|---|
| Clone | | | MW | Swiss- | Prot Mw | Mw | (Prot | Unigene |
| ID | Accession | Protein | (kDa) | Prot Mw | plus tag | (+MYKKAGT) | Param) | ID |
| IOH6288 | BC006233.2 | AAH06233.1 | 32749.02 | 32744.3 | 33524.3 | 33546.9 | 6.01 | Hs.159525 |
| IOH23101 | NM_145691.3 | NP_663729.1 | 32776.95 | 32772.3 | 33552.3 | 33574.9 | 7.68 | Hs.13434 |
| IOH42083 | NM_199123.1 | NP_954574.1 | 33705.87 | 33701.2 | 34481.2 | 34503.8 | 7.66 | Hs.510407 |
| IOH13536 | NM_005842.2 | NP_005833.1 | 34693.31 | 34688.3 | 35468.3 | 35491.2 | 8.88 | Hs.18676 |
| IOH40625 | NM_138416.1 | NP_612425.1 | 35394.89 | 35389.8 | 36169.8 | 36192.8 | 9.46 | Hs.270778 |
| IOH26161 | NM_032288.5 | NP_115664.2 | 35823.33 | 35818.2 | 36598.2 | 36621.2 | 11.73 | Hs.277533 |
| IOH9779 | NM_153273.3 | NP_695005.1 | 50242.38 | 50235.4 | 51015.4 | 51040.3 | 7.27 | Hs.555908 |
| IOH3261 | NM_001007245.1 | NP_001007246.1 | 50275.63 | 50268.5 | 51048.5 | 51073.5 | 7.50 | Hs.7879 |
| IOH10404 | NM_004751.1 | NP_004742.1 | 50870.51 | 50863.5 | 51643.5 | 51668.4 | 8.70 | Hs.194710 |
| IOH11418 | NM_001415.2 | NP_001406.1 | 51117.09 | 51109.4 | 51889.4 | 51915.0 | 8.83 | Hs.539684 |
| IOH29386 | BC051332.1 | AAH51332.1 | 70018.54 | 70008.7 | 70788.7 | 70816.4 | 5.77 | Hs.76530 |
| IOH12674 | BC021301.2 | AAH21301.1 | 73279.32 | 73269.1 | 74049.1 | 74077.2 | 9.11 | Hs.552580 |
| IOH11193 | BC024178.1 | AAH24178.1 | 73358.47 | 73348.2 | 74128.2 | 74156.4 | 5.49 | Hs.128258 |
| IOH40892 | NM_033495.2 | NP_277030.2 | 73878.3 | 73867.9 | 74647.9 | 74676.2 | 6.37 | Hs.348262 |
| IOH46182 | BC006206.2 | AAH06206.3 | 100943.8 | 100929.3 | 101709.3 | 101741.7 | 6.37 | Hs.26814 |
| IOH3655 | NM_004990.2 | NP_004981.2 | 101130.1 | 101115.7 | 101895.7 | 101928.0 | 5.96 | Hs.355867 |
| IOH40838 | BC067089.1 | AAH67089.1 | 105825.2 | 105809.9 | 106589.9 | 106623.1 | 5.91 | Hs.475525 |
| IOH26721 | NM_003248.3 | NP_003239.2 | 105884.4 | 105869.2 | 106649.2 | 106682.3 | 4.46 | Hs.211426 |
| IOH29199 | NM_015187.1 | NP_056002.1 | 111727.5 | 111711.9 | 112491.9 | 112525.4 | 6.36 | Hs.479384 |
| IOH26205 | NM_003325.3 | NP_003316.3 | 111851.1 | 111835 | 112615 | 112649.0 | 8.52 | Hs.474206 |

The proteins were expressed in E. coli under conditions that maximize inclusion body formation. The expression system resulted in an N-terminal extension of seven amino acids (MYKKAGT, SEQ ID NO:133), followed by the initiator methionine encoded by the ORF. The 20 proteins were purified by preparative SDS PAGE or 2D-LC (anion exchange and reversed phase) to >95% purity. Trypsin digestion of the purified constructs results in the generation of a tripeptide (MYK) plus free K, or a tetrapeptide (MYKK, SEQ ID NO:134) resulting from 1 missed cleavage and an N-terminal extension of 3 (AGT) or 4 (KAGT, SEQ ID NO:135, 1 missed cleavage) amino acids. The proteins were mixed in equimolar amounts (5 picomoles per protein). Contaminants did not exceed 1% in the final mixture.

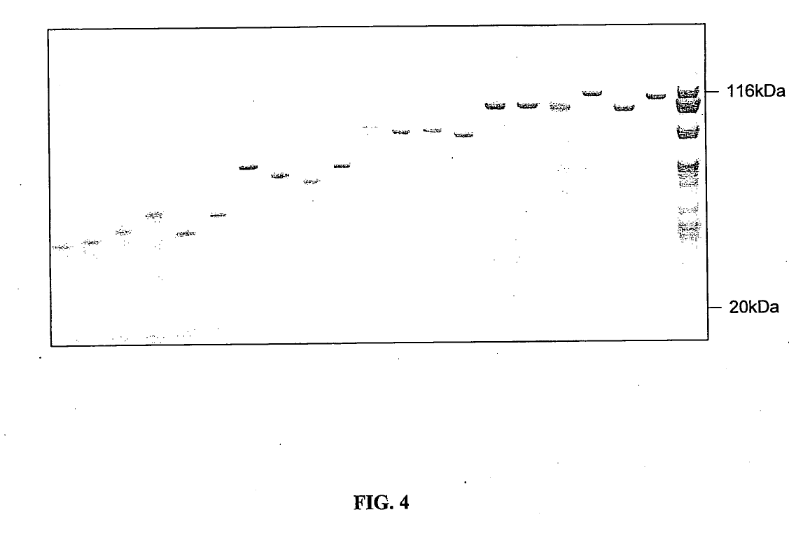

The purified proteins were analyzed by SDS PAGE individually (FIG. 4) and after blending. All proteins loaded individually at 5 pmol/protein are also included in the blend at 5 pmol/protein.

Co-migration of multiple proteins in the blend is a feature designed to simulate biological complexity. Variation in the staining intensity of protein bands may be due to inherent protein-to-protein variation in Coomassie staining and/or BCA assay quantification.

Protein Expression: Overnight starter cultures of the expression host BL21 Star™ (DE3) were used to inoculate larger expression cultures. Expression cultures were grown at 37° C. to an A600 nm of 0.5-0.6 and induced with 1 mM IPTG. Growth at 37° C. continued for 3-3.5 hours before harvesting cells. Cell pellets were stored at −20° C. until use.

Protein Purification: Cell pellets were lysed in BugBuster lysis reagent containing 50 U/mL Benzonase (Novagen). The insoluble pellet was repeatedly washed by resuspension and centrifugation in buffer containing 1% Triton X-100. Final inclusion body pellets were washed in buffer without Triton X-100 before storage at 4° C.

Proteins were further purified from inclusion bodies either by preparative SDS PAGE or by 2D-LC (anion exchange and reverse phase) under denaturing conditions. Protein purity was determined by SDS PAGE analysis. Pure fractions were pooled and concentrated by centrifugal ultrafiltration prior to acetone precipitation. Protein quantification was performed on resuspended protein pellets (1% SDS, 2 mM DTT) using a reducing agent compatible BCA assay (Pierce).

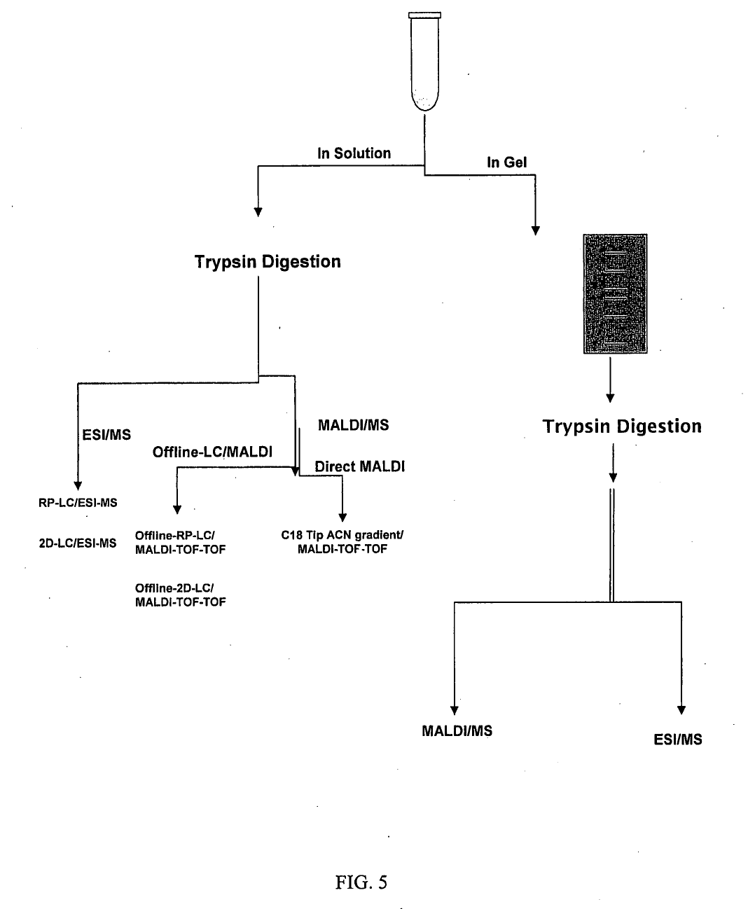

In Solution Trypsin Digestion and Mass Spectrometry: The dry protein standards pellet was dissolved in 20 μL of 8.0M urea before the addition of 80 μL of 25 mM NH₄HCO₃, pH 8.0. 1 μL of trypsin solution (0.2 μg/μL prepared in 25 mM NH₄HCO₃, pH 8.0) was added to the mixture and incubated at 37° C. overnight. The digested peptide mixture was analyzed using both MALDI/TOF-TOF 4700 Proteomics Analyzer (Applied Biosystems) and nano-LC/ESI-MS Q-TOF Premier, and Q-TOF API-US (Waters). To simplify the complexity of the mixture for MALDI/TOF-TOF analysis, the sample was fractionated with a C18 tip using increasing concentrations of acetonitrile (ACN) to elute the peptides. Super saturated α-CHCA dissolved in 50% ACN/0.1% TFA was used as matrix. These protocols are depicted schematically on the left side of FIG. 5.

In Gel Trypsin Digestion and Mass Spectrometry: The dry protein standards pellet was dissolved in LDS sample buffer and heated at 70° C. for 10 minutes in the presence of 50 mM DTT. The entire sample (100 pmol total protein) was then fractionated by SDS PAGE, 10% Bis-Tris NuPAGE® gel (Invitrogen).

Gel spots were excised and washed in 50% ACN in 25 mM ammonium bicarbonate buffer until clear, then dehydrated in CAN and dried in a SpeedVac. Gel plugs were rehydrated with a minimal volume of trypsin solution (10 μg/ml in 25 mM ammonium bicarbonate buffer) and incubated overnight at 37° C. The digested peptides were extracted from the gel in two steps. The first extract was collected after incubating the gel pieces in 25 μl of 5% TFA for 30 minutes at room temperature. The second extract was collected after incubating the gel pieces in 25 μl of 5% TFA/ACN for 30 minutes at room temperature. The two extracts were pooled and dried in a SpeedVac. The dried extracted peptides were reconstituted in a 50% ACN/0.1% TFA solution for MALDI analysis and 20% ACN/0.1% FA solution for nano-LC/ESI-MS analysis.

The digested peptide mixture was analyzed with both MALDI/TOFTOF 4700 Proteomics Analyzer (Applied Biosystems) and nano-LC/ESI-MS Q-TOF Premier, and Q-TOF API-US (Waters). These protocols are depicted schematically on the right side of FIG. 5.

In-gel analysis was performed with 5 pmol/protein. In-solution analyses were performed with 200 fmol/protein. Raw data files from the Q-TOF instrument were processed with Mascot Distiller (Version 2.1., Matrix Science, London) without smoothing using charge states determined from the MS scans (Perkins et al. (1999) Electrophoresis 20: 3551-3567). The resulting centroid files were searched against the Apr. 15, 2006 NCBInr database with the Mascot search algorithm (Version 2.1) allowing a maximum of one missed trypsin cleavage event and variable modifications that included only Me oxidation. Searches were with and with out restriction to Homo Sapiens species entries. The mass tolerance of the precursor peptide ion was fixed at 200 ppm whereas the mass tolerance for the MS/MS fragment ions was set to 0.5 Da.

To compile the summary of identified proteins, we employed the Protein Prophet and Peptide Prophet algorithms, as implemented in version 1.05 of Scaffold (Proteome Software, Portland, Oreg.) (Nesvizhskii et al. (2003) Anal. Chem. 75: 4646-4658; Keller et al. (2002) Anal. Chem. 74: 5383-92). We required 95% confidence for individual peptides and an minimum protein confidence of 80%. A similar number of proteins were identified using a threshold model for Mascot scores. In brief, a difference of 10 between the peptide ion score and identity score was required using a significance threshold p<0.05. We also set minimum peptide threshold scores for +1, +2, +3 and +4 charge states at 20, 35, 40 and 40, respectively. Raw data files from the 4700 TOF/TOF, without smoothing or baseline correction, were processed with GPS explorer (Version 2.0, Applied Biosystems) and searched against NCBI nr (Aug. 15, 2004) data base with one miss cleavage allowed. The mass tolerance for MS and MS/MSfragment ions was set at 500 ppm and 0.5 Da, respectively.

In-solution tryptic digests of blended protein standards were analyzed by LC ESI Q-TOF MS with decreasing sample injections. Total protein amount injected ranged from 5 pmol to 500 fmol which showed a corresponding decrease in base peak intensity (BPI).

Protein identifications of all twenty proteins in the blend were made in duplicate experiments at the 2 pmol total protein load (100 fmol/protein).

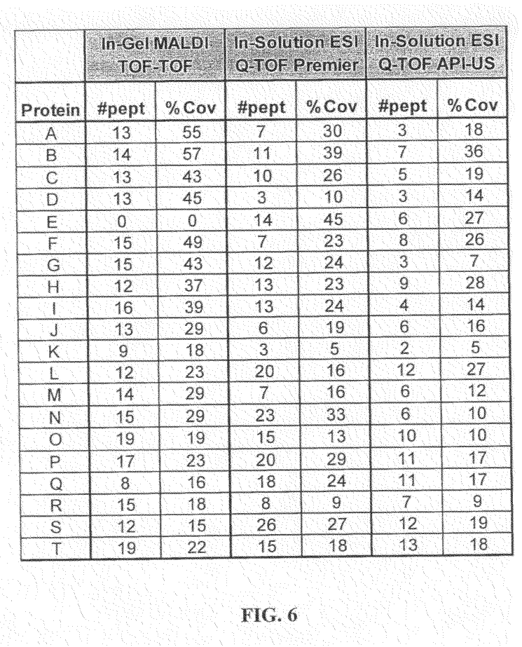

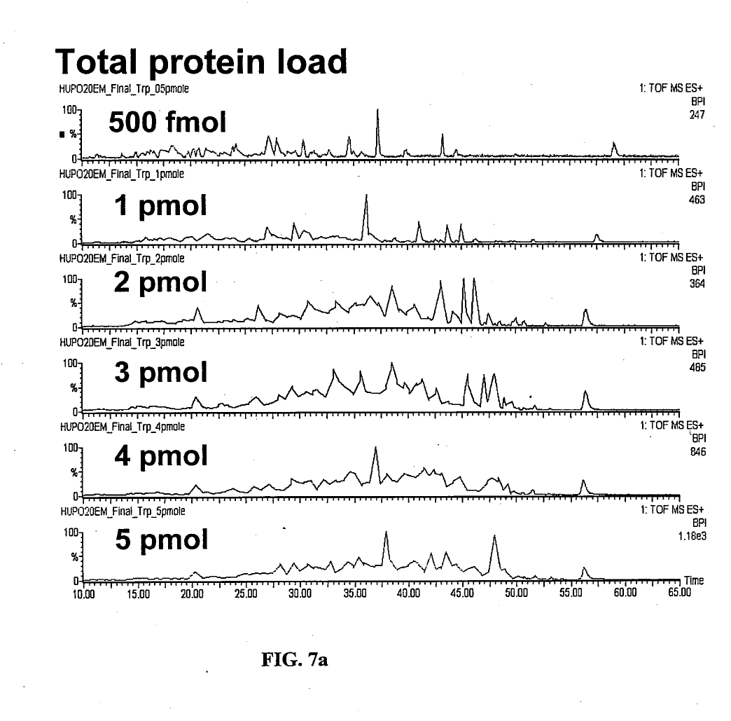

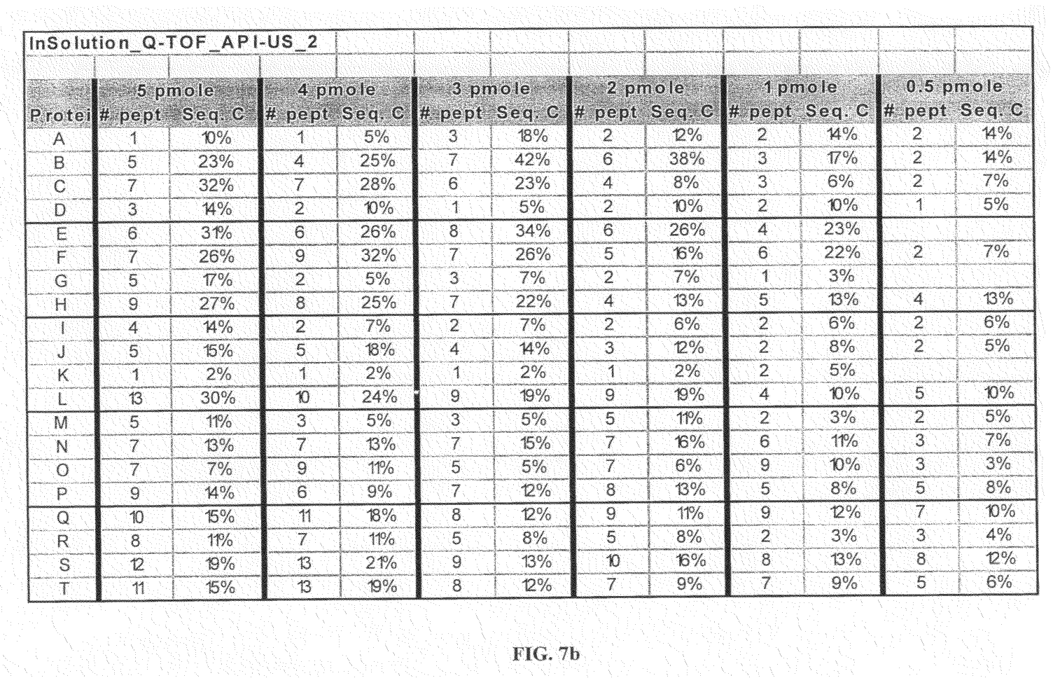

Results of the analyses of the 20 protein Equimolar Proteome Standard Set are provided in FIGS. 6 and 7. (The null result for protein E (FIG. 6) for the ‘In-Gel MALDI TOF-TOF’ analysis was due to searching the wrong data base in which an exact match was not present for the protein.)

The twenty human ORFs selected to mimic the diversity and complexity of human proteome samples were identified by mass spectrometry with no other significant human protein database matches. The recombinant expression and purification strategy employed will allow continued production and batch-to-batch consistency. LC ESI Q-TOF analysis of the protein standard blend identified all twenty proteins from as little as 50 fmol/protein.

### EXAMPLE 7

### Stability of the 20 Protein Proteome Standard Set

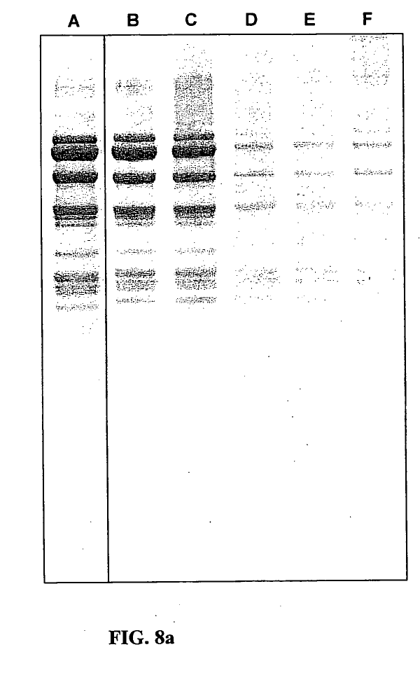

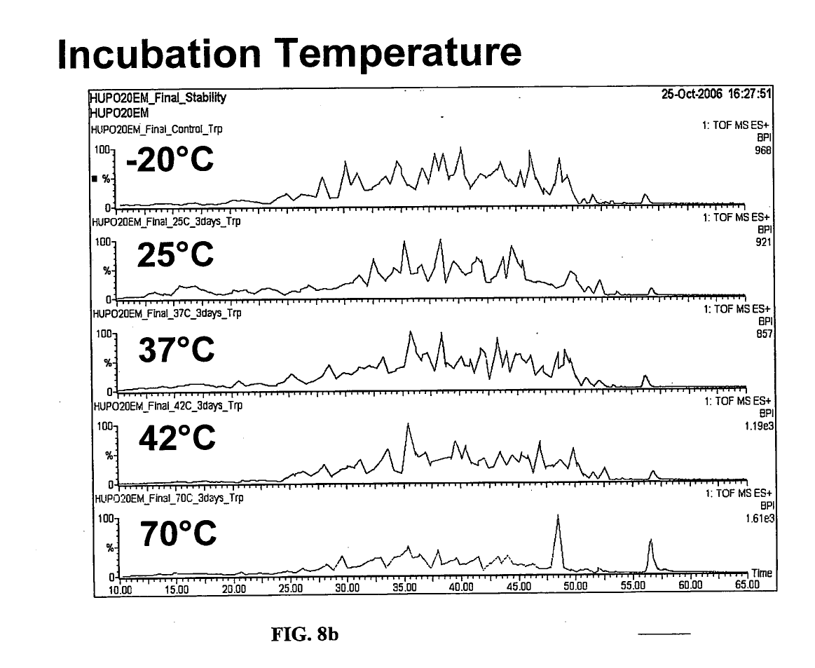

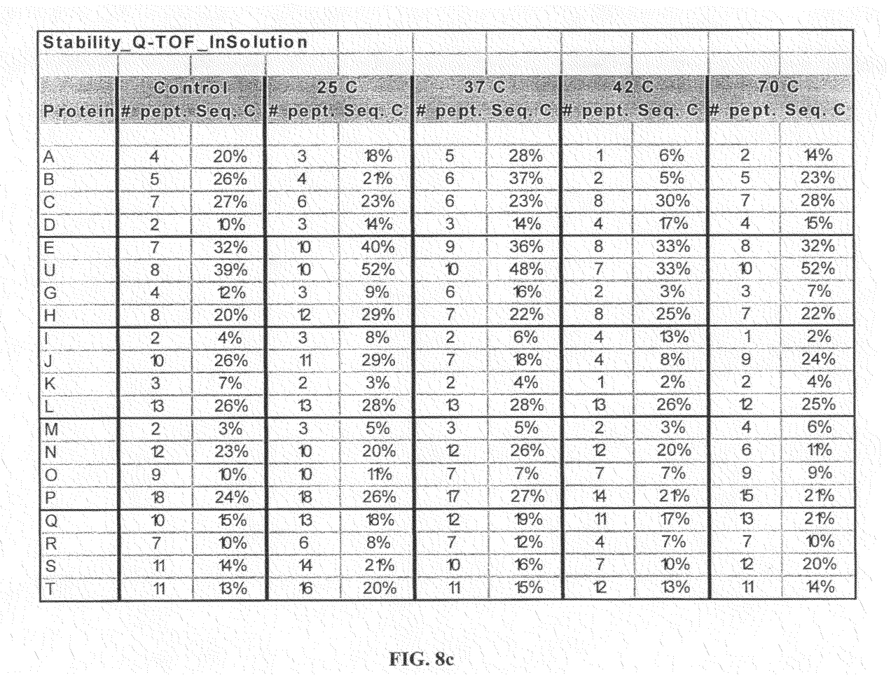

Protein stability was investigated by incubating the blend of protein standards at −20° C., 25° C., 37° C., 42° C. and 70° C. Samples had been stored at −20° C. for 50 days prior to incubation at the indicated temperatures for 2.7 days. Samples were then analyzed by SDS PAGE and mass spectrometry. FIG. 8a represents two gels run on different days, one run immediately after the protein standard blend was made (lane A) and one run at the completion of elevated temperature incubations (lanes B-F). The incubations were carried out at the indicated temperatures with equivalent −20° C. storage times in parenthesis: A) −20° C. (1 day), B) −20° C. (53 days), C) 25° C. (100 days), D) 37° C. (190 days), E) 42° C. (248 days), and F) 70° C. (1432 days). No evidence of protein degradation was found as shown by the absence of lower MW fragments and the resolution of all protein bands in each lane. The lower protein abundance evident in lanes D-F may be due to severe protein dehydration preventing resolubilization. Duplicate stability studies revealed the same data.

Samples corresponding to those represented on the gel (lanes B-F) were also analyzed by in-solution tryptic digestion followed by LC ESI Q-TOF (FIG. 8b). All twenty proteins were identified for each sample in the stability study with similar numbers of identified peptides and percent coverages (FIG. 8c). Base peak intensity was not adversely affected by the artificial aging. Real-time stability studies (incubation at −20° C. only) will continue in order to avoid problems with dehydration at elevated temperatures.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems and methodologies which are reported in the publications which might be used in connection with the invention. Nothing herein is to be construed as an admission that the invention is not entitled to antedate such disclosure by virtue of prior invention.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A standard set for mass spectrometry, comprising a plurality of proteins, wherein when the plurality of proteins are digested with one or more proteases, no two proteolytic fragments generated by digestion having molecular weights between 700 and 4800 Da are identical in amino acid sequence, and further wherein when the plurality of proteins are digested with the one or more proteases and analyzed by mass spectrometry, at least five proteolytic fragments derived from the plurality of proteins produce mass peaks that differ from one another by less than 10 Da.

2. The standard set of claim 1, wherein the plurality of proteins are from a single species of organism.

3. The proteome standard set of claim 2, comprising at least five proteins of a single species of organism.

4. The proteome standard set of claim 3, comprising at least ten proteins of a single species of organism.

5. The proteome standard set of claim 4, comprising at least ten proteins of a single species of organism, wherein when the plurality of proteins are digested with the protease and analyzed by mass spectrometry, at least ten proteolytic fragments derived from the plurality of proteins produce mass peaks that differ from one another by less than 10 Da.

6. The proteome standard set of claim 5, comprising at least twenty proteins of a single species of organism, wherein when the plurality of proteins are digested with a protease and analyzed by mass spectrometry, at least twenty proteolytic fragments derived from the plurality of proteins that produce mass peaks that differ from one another by less than 10 Da.

7. The proteome standard set of claim 1, comprising at least twenty proteins of a single species, wherein when the plurality of proteins are digested with a protease and analyzed by mass spectrometry, the mass spectrum produced has at least two regions that span between 1200 and 1210 Da and that each have at least five proteolytic fragments derived from the plurality of proteins that produce mass peaks that differ from one another by less than 10 Da.

8. The proteome standard set of claim 7, wherein when the plurality of proteins are digested with a protease at least twenty proteolytic fragments are produced that have a molecular weight from 1200 to 1210 daltons.

9. The proteome standard set of claim 1, comprising between twenty-five and thirty proteins, wherein when the plurality of proteins are digested with a protease and analyzed by mass spectrometry, the mass spectrum produced has at least two regions that each have at least twenty proteolytic fragments derived from the plurality of proteins produce mass peaks that differ from one another by less than 10 Da.

10. The proteome standard set of claim 1, wherein at least two of the proteins have a molecular weight of between about 10 kDa and about 200 kDa.

11. The proteome standard set of claim 1, wherein at least two of the proteins have molecular weights that are within 5 kDa of one another

12. The proteome standard set of claim 1, wherein at least two of the proteins have molecular weights that are within 4 kDa of one another

13. The proteome standard set of claim 1, wherein at least two of the proteins have molecular weights that are within 2 kDa of one another

14. The proteome standard set of claim 1, wherein at least four of the proteins have molecular weights of less than 100 kDa, wherein each of the proteins having a molecular weight of less than 100 kDa differs in molecular weight from at least two other proteins of the set by 4 kDa or less.

15. The proteome standard set of claim 1, wherein at least four of the proteins have molecular weights of 100 kDa or greater, wherein each of the proteins having a molecular weight of 100 kDa or greater differs in molecular weight from at least two other proteins of the set by 15 kDa or less.

16. The proteome standard set of claim 1, wherein at least four of the proteins have molecular weights of between 30 and 40 kDa and differ by 5 kDa or less, wherein at least four of the proteins have molecular weights of between 40 and 60 kDa and differ by 5 kDa or less, wherein at least four of the proteins have molecular weights of between 60 and 80 kDa and differ by 7 kDa or less, and wherein at least four of the proteins have molecular weights of between 80 and 150 kDa and differ by 15 kDa or less.

17. The proteome standard set of claim 2, wherein the species is human, mouse, dog, chimp, gorilla, cow, horse, chicken, zebrafish, pufferfish, or Sacchromyces cerevisae.

18. The proteome standard set of claim 17, wherein the species is human.

19. The proteome standard of claim 1, wherein the plurality of proteins are present at concentrations that are within 10% of one another.

20. The proteome standard of claim 1, wherein the plurality of proteins are present at concentrations that are within 5% of one another.

21. The proteome standard of claim 1, wherein the plurality of proteins are present at concentrations that are within 2% of one another.

22. The proteome standard of claim 1, wherein at least one protein of the plurality of proteins are present at a concentrations that is 10% or less than the concentration of at least one other protein of the plurality of proteins.

23. The proteome standard of claim 1, wherein at least one protein of the plurality of proteins are present at a concentrations that is 5% or less than the concentration of at least one other protein of the plurality of proteins.

24. The proteome standard of claim 1, wherein at least one protein of the plurality of proteins are present at a concentrations that is 1% or less than the concentration of at least one other protein of the plurality of proteins.

25. A polypeptide standard set for mass spectrometry comprising a plurality of polypeptides, wherein each polypeptide of the plurality of polypeptides, comprises unique 700 to 4800 Da peptide segments bordered by a protease cleavage site, and further wherein the plurality of polypeptides comprise at least five unique fragments bordered by cleavage sites of the protease that differ from one another by less than 10 Da.

26. The proteome standard set of claim 30, wherein the polypeptides have at least five peptide segments bordered by protease cleavage sites, of each of a first and a second molecular weight range, wherein each of the at least five proteolytic fragments differs from one another by less than 10 Da.

27. A kit comprising two polypeptide standard sets for mass spectrometry,
a) a first standard set comprising a plurality of polypeptides, wherein each polypeptide of the plurality of polypeptides, comprises unique 700 to 4800 Da peptide segments bordered by protease cleavage sites, and further wherein the plurality of polypeptides comprise at least five unique fragments bordered by cleavage sites of the protease that differ from one another by less than 10 Da; and
b) A second standard set comprising the plurality of proteins, wherein at least one of the plurality of proteins is present at a different concentration in the second standard set than the first standard set.

28. The kit of claim 27, further comprising instructions.

29. A method for standardizing laboratories and/or laboratory procedures, comprising separating the proteins of claim 1 on a gel, isolating a plurality or all of said separated polypeptides or proteins, proteolytically cleaving the isolated separated polypeptides or proteins to generate protease fragments, and analyzing the protease fragments.

30. The method of claim 34, wherein the analysis is performed using mass spectrometry.

31. The method of claim 35, wherein the results or said analysis are compared to a reference set of results, to determine whether said laboratory and/or laboratory procedure meets an objective standard of quality.

32. The method of claim 36, wherein said laboratory and/or laboratory procedure meets the standard of quality where the results of the protease fragment analysis differs from the reference set of results by not more than 5%.

33. A method for certifying a laboratory or laboratory technician, comprising providing said laboratory or technician with the polypeptides of claim 30 or the proteins of claim 1, and wherein said laboratory or technician obtains proteolytically cleaved polypeptides of claim 30 or proteolytically cleaved proteins of claim 1, and analyzes said proteolytically cleaved polypeptides or proteolytically cleaved proteins.

34. The method of claim 38, wherein said laboratory or technician separates the polypeptides or proteins of claim 5 on a gel, isolates a plurality or all of said separated polypeptides or proteins, proteolytically cleaves the isolated separated polypeptides or proteins to generate protease fragments, and analyzes the protease fragments.

35. The method of claim 34, wherein the analysis is performed using mass spectrometry.

36. The method of claim 35, wherein the results or said analysis are compared to a reference set of results, to determine whether said laboratory or technician is certified.

37. The method of claim 36, wherein said laboratory or technician is certified where the results of the protease fragment analysis differs from the reference set of results by not more than 5%.

38. A method of generating revenue by providing a customer with a standard set of claim 1 in exchange for consideration.

39. The method of claim 38, wherein said consideration is money.

40. A method for mass spectrometry analysis, comprising digesting a standard set comprising a plurality of proteins with one or more proteases to generate a set of proteolytic fragments derived from the standard set, and analyzing the set of proteolytic fragments by mass spectrometry, wherein no two proteolytic fragments between 700 to 4800 Da of the set of proteolytic fragments, are identical in amino acid sequence and at least five proteolytic fragments of the set of proteolytic fragments, produce mass peaks that differ from one another by less than 10 Da.
